(19) United States
(12) Patent Application Publication
(10) Pub. No.: US 2011/0262477 A1
(43) Pub. Date: Oct. 27, 2011

(54) Compositions and Methods Related to Bacterial EAP, EMP, and/or ADSA Proteins

(75) Inventors: Alice Cheng, Chicago, IL (US); Vilasack Thammavongsa, Chicago, IL (US); Justin Kern, Mountain View, CA (US); Dominique M. Missiakas, Chicago, IL (US); Olaf Schneewind, Chicago, IL (US)

(73) Assignee: UNIVERSITY OF CHICAGO, Chicago, IL (US)

(21) Appl. No.: 13/122,793

(22) PCT Filed: Oct. 6, 2009
(86) PCT No.: PCT/US09/59648

§ 371 Date: Jun. 29, 2011

Related U.S. Application Data
(60) Provisional application No. 61/103,190, filed on Oct. 6, 2008.
(60) Provisional application No. 61/103,196, filed on Oct. 6, 2008.
(60) Provisional application No. 61/170,779, filed on Apr. 20, 2009.

(51) Int. Cl. A61K 39/085; A61P 31/04; A61P 37/04; C07K 19/00

(52) U.S. Cl. 424/190.1; 424/243.1; 424/197.11; 530/405; 424/200.1

(57) ABSTRACT

The present invention concerns methods and compositions for treating or preventing a bacterial infection, particularly infection by a Staphylococcus bacterium. The invention provides methods and compositions for stimulating an immune response against the bacteria. In certain embodiments, the methods and compositions involve an Eap, Emp and/or AdsA amino acid sequence, or an agent that binds and inhibits the same.

This application claims benefit of priority to U.S. Provisional Application Ser. No. 61/103,196, filed Oct. 6, 2008, U.S. Provisional Application Ser. No. 61/103,190, filed Oct. 6, 2008, and U.S. Provisional Application Ser. No. 61/170,779, filed Apr. 20, 2009, the entire contents of which are hereby incorporated by reference.

### BACKGROUND OF THE INVENTION

I. Field of the Invention

The present invention relates generally to the fields of immunology, microbiology, and pathology. More particularly, it concerns methods and compositions involving bacterial proteins, which can be used to invoke an immune response against the bacteria or provide passive immunotherapy. The proteins include Eap, Emp, bacterial adenosine synthase A (AdsA), and/or peptides or proteins comprising Eap, Emp, and/or AdsA amino acid sequences and antibodies that bind the same.

II. Background

The number of both community acquired and hospital acquired infections have increased over recent years. Hospital acquired (nosocomial) infections are a major cause of morbidity and mortality. In the United States, hospital acquired infections affect more than 2 million patients annually. The most frequent infections are urinary tract infections (33% of the infections), followed by pneumonia (15.5%), surgical site infections (14.8%) and primary bloodstream infections (13%) (Emorl and Gaynes, 1993).

Staphylococcus aureus, Coagulase-negative Staphylococci (mostly Staphylococcus epidermidis), enterococcus spp., Escherichia coli and Pseudomonas aeruginosa are the major nosocomial pathogens. Although those pathogens cause approximately the same number of infections, the severity of the disorders they can produce combined with the frequency of antibiotic resistant isolates balance this ranking towards S. aureus and S. epidermidis as being the most significant nosocomial pathogens.

Staphylococcus epidermidis is a normal skin commensal which is also an important opportunistic pathogen responsible for infections of impaired medical devices and infections at sites of surgery. Medical devices infected by S. epidermidis include cardiac pacemakers, cerebrospinal fluid shunts, continuous ambulatory peritoneal dialysis catheters, orthopedic devices and prosthetic heart valves.

Staphylococcus aureus is the most common cause of nosocomial infections with significant morbidity and mortality. It can cause osteomyelitis, endocarditis, septic arthritis, pneumonia, abscesses and toxic shock syndrome. S. aureus can survive on dry surfaces, increasing the chance of transmission. Any S. aureus infection can cause the staphylococcal scalded skin syndrome, a cutaneous reaction to exotoxin absorbed into the bloodstream. It can also cause a type of septicemia called pyaemia that can be life-threatening. Problematically, methicillin-resistant Staphylococcus aureus (MRSA) has become a major cause of hospital-acquired infections.

S. aureus and S. epidermidis infections are typically treated with antibiotics, with penicillin being the drug of choice, whereas vancomycin is used for methicillin resistant isolates. The percentage of staphylococcal strains exhibiting wide-spectrum resistance to antibiotics has become increasingly prevalent, posing a threat for effective antimicrobial therapy. In addition, the recent emergence of vancomycin resistant S. aureus strain has aroused fears that MRSA strains are emerging and spreading for which no effective therapy is available.

The first generation of vaccines targeted against S. aureus or against the exoproteins it produces have met with limited success (Lee, 1996). There remains a need to develop effective vaccines against staphylococcus infections. Additional compositions for treating staphylococcal infections are also needed.

### SUMMARY OF THE INVENTION

Staphylococcus aureus is the single most frequent cause of bacteremia and soft tissue infection in hospitalized or healthy individuals, and dramatic increases in mortality are attributed to the spread of methicillin-resistant S. aureus (MRSA) strains that are often not susceptible to antibiotic therapy (Klevens et al., 2007; Klevens et al., 2006). Abscesses with characteristic fibrin deposits and massive immune cell infiltrates represent the pathological substrate of staphylococcal infection (Lowy, 1998). Scanning electron microscopy was used to observe biofilm-like structures at the center of staphylococcal abscesses. Genetic analyses revealed that in vitro biofilm formation was correlated with the ability of staphylococci to form abscesses, and the inventors identified envelope proteins that are essential for both processes. When purified and used for immunization of mice, Emp and Eap conferrs protective immunity to staphylococcal infection. Passive immunization using antibodies that bind Eap or antibodies that bind Emp also demonstrates therapeutic effects.

This application describes in one embodiment the use of Emp and/or Eap, or antibodies that bind all or part of Emp or Eap, in methods and compositions for the treatment of bacterial and/or staphylococcal infection. This application also provides an immunogenic composition comprising an Emp and/or Eap antigen or immunogenic fragment thereof. Furthermore, the present invention provides methods and compositions that can be used to treat (e.g., limiting staphylococcal abscess formation and/or persistence in a subject) or prevent bacterial infection. In some cases, methods for stimulating an immune response involve administering to the subject an effective amount of a composition including or encoding all or part of the Emp and/or Eap polypeptide or antigen, and in certain aspects other bacterial proteins. Other bacterial proteins include, but are not limited to (i) a secreted virulence factor, and/or a cell surface protein or peptide, or (ii) a recombinant nucleic acid molecule encoding a secreted virulence factor, and/or a cell surface protein or peptide, and/or (iii) polysaccharides and the like.

In other aspects the subject can be administered an Emp and/or Eap modulator, such as an antibody (e.g., a polyclonal, monoclonal, or single chain antibody or fragment thereof) that binds Emp and/or Eap. An Emp and/or Eap modulator can bind Emp and/or Eap directly. The Emp and/or Eap modulator can be an antibody or cell that binds Emp and/or Eap. An antibody can be an antibody fragment, a humanized antibody, a human antibody, and/or a monoclonal antibody or the like. In certain aspects, the Emp and/or Eap modulator is elicited by providing an Emp and/or Eap peptide that results in the production of an antibody that binds Emp and/or Eap in the subject or a source subject (e.g., donor). The Emp and/or Eap modulator is typically formulated in a pharmaceutically acceptable composition. The Emp and/or Eap modulator composition can further comprise at least one staphylococcal antigen or immunogenic fragment thereof, or antibody that binds such (e.g., EsxA, EsxB, EsaB, EsaC, SdrC, SdrD, Hla, SdrE, IsdA, IsdB, SpA, ClfA, ClfB, IsdC, SasB, SasH (AdsA, Ebh, Coa, vWa, or SasF). Additional staphylococcal antigens that can be used in combination with a Emp and/or Eap modulator include, but are not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein. The staphylococcal antigen or antibody can be administered concurrently with the Emp and/or Eap modulator. The staphylococcal antigen or antibody and the Emp and/or Eap modulator can be administered in the same composition.

Certain embodiments are directed to a therapeutic composition comprising an isolated antibody, or fragment thereof, that binds an Emp and/or Eap antigen, or a fragment thereof, in a pharmaceutically acceptable composition wherein the composition is capable of attenuating a staphylococcus bacterial infection in a subject. The antibody can be a human or humanized antibody. In certain aspects the antibody is a polyclonal antibody, or monoclonal antibody, or single chain antibody, or fragment thereof. An antibody composition can further comprise at least one additional isolated antibody that binds an antigen selected from one or more of a group consisting of an isolated ClfA, ClfB, EsaB, EsaC, EsxA, EsxB, Hla, IsdA, IsdB, IsdC, SasB, SasF, SasH (AdsA), Ebh, Coa, vWa, SdrC, SdrD, SdrE, and SpA antigen, or a fragment thereof. Additional antibodies to a staphylococcal antigen that can be used in combination with a Emp and/or Eap modulator include, but are not limited to antibodies against 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

The Emp and/or Eap modulator can also be a recombinant nucleic acid molecule encoding an Emp and/or Eap peptide. A recombinant nucleic acid molecule can encode the Emp and/or Eap peptide and at least one staphylococcal antigen or immunogenic fragment. A nucleic acid can encode or a polypeptide can comprise a number of antigens including 2, 3, 4, 5, 6, 7, 8, 9, 10 or more of one or more of all or part of Eap, Emp, EsaB, EsaC, EsxA, EsxB, SdrC, SdrD, SdrE, Hla or a variant thereof, IsdA, IsdB, IsdC, ClfA, ClfB, SasB, SasF, SasH (AdsA), Ebh, Coa, vWa, SpA or variants thereof. Nucleic acids can encode additional staphylococcal antigens including, but not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

In certain embodiments the methods and compositions use or include or encode all or part of the Emp and/or Eap polypeptide, peptide, or antigen, as well as antibodies that bind the same. In other aspects Emp and/or Eap may be used in combination with other staphylococcal or bacterial factors such as all or part of EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, SpA, or immunogenic fragment thereof or combinations thereof. Additional staphylococcal antigens that can be used in combination with a Emp and/or Eap modulator include, but are not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein. In certain embodiments, 1, 2, 3, 4, 5, 6, 7, 8, 9, 10 or more of EsaB, EsaC, EsxA, EsxB, SdrC, SdrD, SdrE, Hla or a variant thereof, IsdA, IsdB, IsdC, ClfA, ClfB, SasB, SasF, SasH (AdsA), Ebh, Coa, vWa, or SpA can be specifically excluded or included from a method, a composition, or a formulation of the invention. Additional staphylococcal antigens that can be explicitly excluded include, but are not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U56008341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

Embodiments of the invention include compositions that contain or do not contain a bacterium. A composition may or may not include an attenuated or viable or intact staphylococcal or other bacterium. In certain aspects, the composition comprises a bacterium that is not a Staphylococcal bacterium or does not contain Staphylococci bacteria. In certain embodiments a bacterial composition comprises an isolated or recombinantly expressed Emp and/or Eap polypeptide or a nucleotide encoding the same. In still further aspects, the isolated Emp and/or Eap polypeptide is multimerized, e.g., dimerized. In certain aspects of the invention, a composition comprises multimers of 1, 2, 3, 4, 5, 6, 7, 8, 9, 10 or more isolated cell surface proteins or segments thereof. In a further aspect the other polypeptides or peptides can be expressed or included in a bacterial composition including, but not limited to EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, or SpA or immunogenic fragments thereof. Additional staphylococcal polypeptides that can be expressed or included in a bacterial composition include, but are not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein. Alternatively, the composition may be or include a recombinantly engineered Staphylococcus bacterium that has been altered in a way that comprises specifically altering the bacterium with respect to a secreted virulence factor or cell surface protein. For example, the bacteria may be recombinantly modified to express more of the virulence factor or cell surface protein than it would express if unmodified.

The term “Emp polypeptide” or “Eap polypeptide” refers to polypeptides that include isolated wild-type Emp or Eap proteins from staphylococcus bacteria, as well as variants and segments or fragments that stimulate an immune response against staphylococcus bacteria Emp or Eap proteins. Similarly, the term EsaB, EsaC, EsxA, EsxB, SdrC, SdrD, SdrE, Hla, IsdA, IsdB, ClfA, ClfB, IsdC, SasB, SasF, SasH (AdsA), Ebh, Coa, vWa, or SpA protein refers to a protein that includes isolated wild-type EsaB, EsaC, EsxA, EsxB, SdrC, SdrD, SdrE, Hla or a variant thereof, IsdA, IsdB, ClfA, ClfB, IsdC, SasB, SasF, SasH (AdsA), or SpA polypeptides from staphylococcus bacteria, as well as variants, segments, or fragments that stimulate an immune response against staphylococcus bacteria EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, or SpA proteins. Additionally, the terms 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein refers to a protein that includes isolated wild type 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U56008341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein from staphylococcus bacteria, as well as variants, segments, or fragments that stimulate an immune response against staphylococcal 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein. An immune response refers to a humoral response, a cellular response, or both a humoral and cellular response in an organism. An immune response can be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines

Embodiments of the present invention include methods for eliciting an immune response against a staphylococcus bacterium or staphylococci in a subject comprising providing to the subject an effective amount of 1, 2, 3, 4, 5, 6, 7, 8, 9, 10 or more antigens or segments/fragments thereof. Staphylococcal antigens include, but are not limited to an Eap, Emp, EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, or SpA, or a segment, fragment, or immunogenic fragment thereof. Additional Staphylococcal antigens include, but are not limited to 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

In certain embodiments Emp and/or Eap polypeptides or immunogenic fragments thereof can be provided in combination with one or more antigens or immunogenic fragments of one or more of EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, or SpA. Additional antigens or immunogenic fragments of 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein can also be used.

Embodiments of the invention include compositions that may include a polypeptide, peptide, or protein that has or has at least 70%, 75%, 80%, 85%, 90%, 95%, 96%, 97%, 98%, or 99% identity or similarity to Emp, Eap EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U56008341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein over 5, 10, 15, 20, 50, 100, 200, or more consecutive amino acids including all values and ranges there between. In a further embodiment of the invention a composition may include a polypeptide, peptide, or protein that is or is at least 70%, 75%, 80%, 85%, 90%, 95%, 96%, 97%, 98%, or 99% identical or similar to an Emp polypeptide (SEQ ID NO:2, 50-53) and/or Eap polypeptide (SEQ ID NO:4) or Emp nucleic acid (SEQ ID NO:1) and/or Eap nucleic acid (SEQ ID NO:3). In certain aspects the Emp polypeptide or Eap polypeptide will have an amino acid sequence of (SEQ ID NO:2) or (SEQ ID NO:4), respectively. Similarity or identity, with identity being preferred, is known in the art and a number of different programs can be used to identify whether a protein (or nucleic acid) has sequence identity or similarity to a known sequence. Sequence identity and/or similarity is determined using standard techniques known in the art, including, but not limited to, the local sequence identity algorithm of Smith & Waterman (1981), by the sequence identity alignment algorithm of Needleman & Wunsch (1970), by the search for similarity method of Pearson & Lipman (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (1984), preferably using the default settings, or by inspection. Preferably, percent identity is calculated by using alignment tools known to and readily ascertainable to those of skill in the art.

The term “AdsA polypeptide” refers to polypeptides that include isolated wild-type bacterial AdsA proteins, e.g., staphylococcus (S. aureus SEQ ID NO:36) or bacillus (B. anthracis SEQ ID NO:41) bacteria, as well as variants and segments or fragments of AdsA proteins. In certain aspects, the AdsA polypeptide stimulates an immune response against bacterial AdsA proteins.

In still further embodiments of the invention a composition may include a polypeptide, peptide, or protein that is or is at least 70%, 75%, 80%, 85%, 90%, 95%, 96%, 97%, 98%, or 99% identical or similar to an EsxA protein. In certain aspects the EsxA protein will have the amino acid sequence of SEQ ID NO:6.

In still further embodiments of the invention a composition may include a polypeptide, peptide, or protein that is or is at least 70%, 75%, 80%, 85%, 90%, 95%, 96%, 97%, 98%, or 99% identical or similar to an EsxB protein. In certain aspects the EsxB protein will have the amino acid sequence of SEQ ID NO:8.

In yet still further embodiments of the invention a composition may include a polypeptide, peptide, or protein that is or is at least 70%, 75%, 80%, 85%, 90%, 95%, 96%, 97%, 98%, or 99% identical or similar to an SdrD protein. In certain aspects the SdrD protein will have the amino acid sequence of SEQ ID NO:10.

In further embodiments of the invention a composition may include a polypeptide, peptide, or protein that is or is at least 70%, 75%, 80%, 85%, 90%, 95%, 96%, 97%, 98%, or 99% identical or similar to an SdrE protein. In certain aspects the SdrE protein will have the amino acid sequence of SEQ ID NO:12.

In still further embodiments of the invention a composition may include a polypeptide, peptide, or protein that is or is at least 70%, 75%, 80%, 85%, 90%, 95%, 96%, 97%, 98%, or 99% identical or similar to an IsdA protein. In certain aspects the IsdA protein will have the amino acid sequence of SEQ ID NO:14.

In yet still further embodiments of the invention a composition may include a polypeptide, peptide, or protein that is or is at least 70%, 75%, 80%, 85%, 90%, 95%, 96%, 97%, 98%, or 99% identical or similar to an IsdB protein. In certain aspects the IsdB protein will have the amino acid sequence of SEQ ID NO:16.

Embodiments of the invention include compositions that include a polypeptide, peptide, or protein that is or is at least 70%, 75%, 80%, 85%, 90%, 95%, 96%, 97%, 98%, or 99% identical or similar to a SpA protein. In certain aspects the SpA protein will have the amino acid sequence of SEQ ID NO:18.

In a further embodiments of the invention a composition may include a polypeptide, peptide, or protein that is or is at least 70%, 75%, 80%, 85%, 90%, 95%, 96%, 97%, 98%, or 99% identical or similar to a ClfB protein. In certain aspects the ClfB protein will have the amino acid sequence of SEQ ID NO:20.

In still further embodiments of the invention a composition may include a polypeptide, peptide, or protein that is or is at least 70%, 75%, 80%, 85%, 90%, 95%, 96%, 97%, 98%, or 99% identical or similar to an IsdC protein. In certain aspects the IsdC protein will have the amino acid sequence of SEQ ID NO:22.

In yet further embodiments of the invention a composition may include a polypeptide, peptide, or protein that is or is at least 70%, 75%, 80%, 85%, 90%, 95%, 96%, 97%, 98%, or 99% identical or similar to a SasF protein. In certain aspects the SasF protein will have the amino acid sequence of SEQ ID NO:24.

In yet still further embodiments of the invention a composition may include a polypeptide, peptide, or protein that is or is at least 70%, 75%, 80%, 85%, 90%, 95%, 96%, 97%, 98%, or 99% identical or similar to an SdrC protein. In certain aspects the SdrC protein will have the amino acid sequence of SEQ ID NO:26.

In yet still further embodiments of the invention a composition may include a polypeptide, peptide, or protein that is or is at least 70%, 75%, 80%, 85%, 90%, 95%, 96%, 97%, 98%, or 99% identical or similar to an ClfA protein. In certain aspects the ClfA protein will have the amino acid sequence of SEQ ID NO: 28.

In yet still further embodiments of the invention a composition may include a polypeptide, peptide, or protein that is or is at least 70%, 75%, 80%, 85%, 90%, 95%, 96%, 97%, 98%, or 99% identical or similar to an EsaB protein. In certain aspects the EsaB protein will have the amino acid sequence of SEQ ID NO: 30.

In yet still further embodiments of the invention a composition may include a polypeptide, peptide, or protein that is or is at least 70%, 75%, 80%, 85%, 90%, 95%, 96%, 97%, 98%, or 99% identical or similar to an EsaC protein. In certain aspects the EsaC protein will have the amino acid sequence of SEQ ID NO: 32.

In yet still further embodiments of the invention a composition may include a polypeptide, peptide, or protein that is or is at least 70%, 75%, 80%, 85%, 90%, 95%, 96%, 97%, 98%, or 99% identical or similar to an SasB protein. In certain aspects the SasB protein will have the amino acid sequence of SEQ ID NO: 34.

In yet still further embodiments of the invention a composition may include a polypeptide, peptide, or protein that is or is at least 70%, 75%, 80%, 85%, 90%, 95%, 96%, 97%, 98%, or 99% identical or similar to an SasH (AdsA) protein. In certain aspects the SasH (AdsA) protein will have the amino acid sequence of SEQ ID NO: 36 or SEQ ID NO:41.

In yet still further embodiments of the invention a composition may include a polypeptide, peptide, or protein that is or is at least 70%, 75%, 80%, 85%, 90%, 95%, 96%, 97%, 98%, or 99% identical or similar to an Hla protein. In certain aspects the Hla protein will have the amino acid sequence of SEQ ID NO: 37. In certain aspects, a variant Hla includes amino acid substitutions or D24C, H35C, H35K, H35L, R66c, E70C, or K110C, or any combination thereof (amino acids referred to using single letter code).

In yet still further embodiments of the invention a composition may include a polypeptide, peptide, or protein that is or is at least 70%, 75%, 80%, 85%, 90%, 95%, 96%, 97%, 98%, or 99% identical or similar to an Ebh protein. In certain aspects the Ebh protein will have the amino acid sequence of SEQ ID NO:38.

In yet still further embodiments of the invention a composition may include a polypeptide, peptide, or protein that is or is at least 70%, 75%, 80%, 85%, 90%, 95%, 96%, 97%, 98%, or 99% identical or similar to an Coa protein. In certain aspects the Coa protein will have the amino acid sequence of SEQ ID NO:39.

In yet still further embodiments of the invention a composition may include a polypeptide, peptide, or protein that is or is at least 70%, 75%, 80%, 85%, 90%, 95%, 96%, 97%, 98%, or 99% identical or similar to an vWa protein. In certain aspects the vWa protein will have the amino acid sequence of SEQ ID NO: 40.

In certain aspects, a polypeptide or segment/fragment can have a sequence that is at least 85%, at least 90%, at least 95%, at least 98% or at least 99% or more identical to the amino acid sequence of the reference polypeptide. The term “similarity” refers to a polypeptide that has a sequence that has a certain percentage of amino acids that are either identical with the reference polypeptide or constitute conservative substitutions with the reference polypeptides.

The polypeptides or segments or fragments described herein may include the following, or at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 350, 400, 450, 500, 550 or more contiguous amino acids, or any range derivable therein, of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:37, SEQ ID NO:38, SEQ ID NO:39, SEQ ID NO:40, SEQ ID NO:41, or SEQ ID NO:41.

In further embodiments a composition comprises a recombinant nucleic acid molecule encoding 1, 2, 3, 4, or more of Eap, Emp, EsaB, EsaC, EsxA, EsxB, SdrC, SdrD, SdrE, Hla or a variant thereof, IsdA, IsdB, ClfA, ClfB, IsdC, SasB, SasF, SasH (AdsA), SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U56008341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

Still further embodiments include methods for stimulating in a subject a protective or therapeutic immune response against a staphylococcus bacterium comprising administering to the subject an effective amount of a composition including (i) an Emp and/or Eap polypeptide or peptide thereof; or, (ii) a nucleic acid molecule encoding an Emp and/or Eap polypeptide or peptide thereof, or (iii) administering an Emp and/or Eap polypeptide with any combination or permutation of bacterial proteins or polysaccharides described herein.

Yet still further embodiments include vaccines comprising a pharmaceutically acceptable composition having an isolated Emp and/or Eap polypeptides, or segment or fragment thereof, or any other combination or permutation of protein(s) or peptide(s) or polysaccharide(s) described, wherein the composition is capable of stimulating an immune response against a staphylococcus bacterium. The vaccine may comprise an isolated Emp and/or Eap polypeptide, and/or any other combination or permutation of protein(s) or peptide(s) or polysaccharide(s) described. In certain aspects of the invention the isolated Emp and/or Eap polypeptide, or any other combination or permutation of protein(s) or peptide(s) or polysaccharide(s) described are multimerized, e.g., dimerized.

In a further aspect, the vaccine composition is contaminated by less than about 10, 9, 8, 7, 6, 5, 4, 3, 2, 1, 0.5, 0.25, 0.05% (or any range derivable therein) of other Staphylococcal proteins. A composition may further comprise an isolated non-Emp and/or non-Eap polypeptide. Typically the vaccine comprises an adjuvant. In certain aspects a protein or peptide of the invention is linked (covalently or non-covalently coupled) to the adjuvant, preferably the adjuvant is chemically conjugated to the protein.

In still yet further embodiments, a vaccine composition is a pharmaceutically acceptable composition having a recombinant nucleic acid encoding all or part of an Emp and/or Eap polypeptide, and/or any other combination or permutation of protein(s) or peptide(s) described, wherein the composition is capable of stimulating an immune response against a staphylococcus bacteria. The vaccine composition may comprise a recombinant nucleic acid encoding all or part of an Emp and/or Eap polypeptide, and/or any other combination or permutation of protein(s) or peptide(s) described. In certain embodiments the recombinant nucleic acid contains a heterologous promoter. Preferably the recombinant nucleic acid is a vector. More preferably the vector is a plasmid or a viral vector.

Still further embodiments include methods for stimulating in a subject a protective or therapeutic immune response against a staphylococcus bacterium comprising administering to the subject an effective amount of a composition of an Emp and/or Eap polypeptide or segment/fragment thereof comprising one or more of (i) a EsaB, EsaC, EsxA, EsxB, SdrC, SdrD, SdrE, Hla or a variant thereof, IsdA, IsdB, ClfA, ClfB, IsdC, SasB, SasF, SasH (AdsA), SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein polypeptide or segment or fragment thereof; or, (ii) a nucleic acid molecule encoding the same. Methods of the invention also include Emp and/or Eap compositions that contain 1, 2, 3, 4, 5, 6, 7, 8, 9, 10 or more of EsaB, EsaC, EsxA, EsxB, SdrC, SdrD, SdrE, Hla or a variant thereof, IsdA, IsdB, ClfA, ClfB, IsdC, SasB, SasF, SasH (AdsA), SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein in various combinations. In certain aspects a vaccine formulation includes an IsdA polypeptide or segment or fragment thereof.

In still a further aspect the invention includes a staphylococcal bacterium lacking an Emp and/or Eap polypeptide and/or EsaB polypeptide. Such a bacterium will be limited or attenuated with respect to prolonged or persistent abscess formation and/or biofilm formation. This characteristic can be used to provide bacterial strains for the production of attenuated bacteria for use in the preparation of vaccines or treatments for staphylococcal infections or related diseases. In yet a further aspect, Emp and/or Eap can be overexpressed in an attenuated bacterium to further enhance or supplement an immune response or vaccine formulation.

Any embodiment discussed with respect to one aspect of the invention applies to other aspects of the invention as well. In particular, any embodiment discussed in the context of an Emp and/or Eap peptide or nucleic acid may be implemented with respect to EsaB, EsaC, EsxA, EsxB, SdrC, SdrD, SdrE, Hla or a variant thereof, IsdA, IsdB, ClfA, ClfB, IsdC, SasB, SasF, SasH (AdsA), SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding proteins or nucleic acids or antibodies that bind the same, and vice versa.

The inventors have examined the ability of S. aureus to escape phagocytic clearance in blood and identified adenosine synthase A (AdsA), a cell wall anchored enzyme that converts adenosine monophosphate to adenosine, as a critical virulence factor. Staphylococcal synthesis of adenosine in blood, escape from phagocytic clearance, and subsequent formation of organ abscesses were all dependent on adsA and could be rescued by an exogenous supply of adenosine. An AdsA homolog was identified in the anthrax pathogen and adenosine synthesis also enabled escape of Bacillus anthracis from phagocytic clearance. Taken together, these results suggest that staphylococci and other bacterial pathogens exploit the immunomodulatory attributes of adenosine to escape host immune responses. Certain embodiments of the invention are based on the discovery that the synthesis of the signaling molecule adenosine is immunosuppressive and modulation of its synthesis activity can be exploited for therapeutic purposes.

This application describes in one embodiment the use of AdsA, or antibodies that bind all or part of AdsA, or inhibitors of AdsA activity in methods and compositions for the treatment of bacterial and/or staphylococcal infection. This application also provides an immunogenic composition comprising an AdsA antigen or immunogenic fragment thereof. Furthermore, the present invention provides methods and compositions that can be used to treat (e.g., modulating phagocytic uptake of bacteria) or prevent bacterial infection. In some cases, methods for stimulating an immune response involve administering to the subject an effective amount of a composition including or encoding all or part of a bacterial AdsA polypeptide or antigen, and in certain aspects other bacterial proteins and bacterial polysaccharides. Other bacterial proteins include, but are not limited to (i) a secreted virulence factor, and/or a cell surface protein or peptide, or (ii) a recombinant nucleic acid molecule encoding a secreted virulence factor, and/or a cell surface protein or peptide.

In other aspects the subject can be administered an AdsA modulator, such as an antibody (e.g., a polyclonal, monoclonal, or single chain antibody or fragment thereof) that binds AdsA or a small molecule that inhibits AdsA activity or stability. An AdsA modulator may bind AdsA directly. The AdsA modulator can be an antibody or cell that binds AdsA. An antibody can be an antibody fragment, a humanized antibody, a human antibody, and/or a monoclonal antibody or the like. In certain aspects, the AdsA modulator is elicited by providing an AdsA peptide or a bacteria expressing the same that results in the production of an antibody that binds AdsA in the subject. The AdsA modulator is typically formulated in a pharmaceutically acceptable composition. The AdsA modulator composition can further comprise at least one staphylococcal antigen or immunogenic fragment thereof, or antibody that bind such (e.g., Eap, Emp, EsaB, EsaC, EsxA, EsxB, SasB, SdrC, SdrD, SdrE, Hla, IsdA, IsdB, Spa, ClfA, ClfB, IsdC, Coa, Ebh, vWa or SasF). The staphylococcal antigen or antibody can be administered concurrently with the AdsA modulator. An antigen and/or antibody and/or antibiotic, and an AdsA modulator can be administered in the same composition.

Certain embodiments are directed to a therapeutic composition comprising an isolated antibody, or fragment thereof, that binds an AdsA protein or antigen, or a fragment thereof, in a pharmaceutically acceptable composition wherein the composition is capable of attenuating a staphylococcus bacterial infection in a subject, e.g., modulating phagocytic uptake of bacteria. The modulator can be a small molecule, such as an adenosine analog. The antibody can be a human or humanized antibody. In certain aspects the antibody is a polyclonal antibody, or monoclonal antibody, or single chain antibody, or fragment thereof.

An antibody composition can further comprise at least one additional isolated antibody that binds a antigen selected from a group consisting of an isolated ClfA, ClfB, Eap, Emp, EsaB, EsaC, EsxA, EsxB, Hla, IsdA, IsdB, IsdC, SasB, SasF, SdrC, SdrD, Coa, Ebh, vWa, SdrE, and SpA antigen, or a fragment thereof.

The AdsA modulator can also be a recombinant nucleic acid molecule encoding an AdsA peptide. A recombinant nucleic acid molecule can encode the AdsA peptide and/or at least one staphylococcal antigen or immunogenic fragment. A nucleic acid can encode or a polypeptide can comprise a number of antigens including 2, 3, 4, 5, 6, 7, 8, 9, 10 or more of one or more of all or part of AdsA (SasH), Eap, Emp, EsaB, EsaC, EsxA, EsxB, SdrC, SdrD, SdrE, Coa, Ebh, vWa, Hla or a variant thereof, IsdA, IsdB, ClfA, ClfB, IsdC, SasB, SasF, or SpA.

The AdsA modulator can also be a recombinant nucleic acid molecule encoding an AdsA peptide. A recombinant nucleic acid molecule can encode the AdsA peptide and/or at least one staphylococcal antigen or immunogenic fragment. A nucleic acid can encode or a polypeptide can comprise a number of antigens including 2, 3, 4, 5, 6, 7, 8, 9, 10 or more of one or more of all or part of AdsA (SasH), Eap, Emp, EsaB, EsaC, EsxA, EsxB, SdrC, SdrD, SdrE, Coa, Ebh, vWa, Hla or a variant thereof, IsdA, IsdB, ClfA, ClfB, IsdC, SasB, SasF, or SpA.

Embodiments of the invention include compositions that contain or do not contain a bacterium. A composition may or may not include an attenuated or viable or intact staphylococcal or other bacterium. In certain aspects, the composition comprises a bacterium that is not a Staphylococci bacterium or does not contain Staphylococci bacteria. In certain embodiments a bacterial composition comprises an isolated or recombinantly expressed AdsA polypeptide or a nucleic acid encoding the same. In still further aspects, the isolated AdsA polypeptide is multimerized, e.g., dimerized. In certain aspects of the invention, a composition comprises multimers of 1, 2, 3, 4, 5, 6, 7, 8, 9, 10 or more isolated cell surface proteins or segments thereof. In a further aspect the other polypeptides or peptides can be expressed or included in a bacterial composition including, but not limited to AdsA, Eap, Emp, EsaB, EsaC, EsxA, EsxB, SdrC, Coa, Ebh, vWa, SdrD, SdrE, Hla or a variant thereof, IsdA, IsdB, ClfA, ClfB, IsdC, SasB, SasF, or SpA or immunogenic fragments thereof. Alternatively, the composition may be or include a recombinantly engineered Staphylococcus bacterium that has been altered in a way that comprises specifically altering the bacterium with respect to a secreted virulence factor or cell surface protein. For example, the bacteria may be recombinantly modified to express more of the virulence factor or cell surface protein than it would express if unmodified.

Similarly, the term Eap, Emp, EsaB, EsaC, EsxA, EsxB, SdrC, SdrD, SdrE, Hla, IsdA, IsdB, ClfA, ClfB, IsdC, SasB, Coa, Ebh, vWa, SasF, or SpA protein refers to a protein that includes the respective isolated wild-type polypeptides from staphylococcus bacteria, as well as variants, segments, or fragments that stimulate an immune response against the same. An immune response refers to a humoral response, a cellular response, or both a humoral and cellular response in an organism. An immune response can be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines Bacterial AdsA polypeptides include, but are not limited to all or part of the amino acid sequences of the following bacteria (accession number): Staphlyococcus aureus (ref|YP_{—}001573948, ref|YP_{—}184935, ref|YP_{—}039500, ref|NP_{—}373261, ref|NP_{—}370547, ref|YP_{—}042156, ref|NP_{—}644838, ref|YP_{—}415541, dbj|BAA82250); Staphlyococcus hemolyticus (ref|YP_{—}254367); Streptococcus sanguinis (ref|YP_{—}001035187); Streptococcus gordonii (ref|YP_{—}001450531); Enterococcus faecalis (ref|NP_{—}813870); Streptococcus suis (dbj|BAB83980, ref|YP_{—}001200571, ref|YP_{—}001198366); Streptococcus mutans (ref|NP_{—}721592); Streptococcus thermophilus (ref|YP_{—}141373, ref|YP_{—}139455); Alkaliphilus metalliredigens (ref|YP_{—}001321391); Clostridium botulinum (ref|YP_{—}001887045, ref|YP_{—}001921966); Paenibacillus (ref|ZP 02846642); Alkaliphilus oremlandii (ref|YP_{—}001512463); Bacillus clausii (ref|YP_{—}174466); Bacillus halodurans (ref|NP_{—}240892); Clostridium difficile (ref|ZP_{—}03126518, ref|ZP_{—}02748384, ref|YP_{—}001089051, ref|ZP_{—}02726436, ref|ZP_{—}01801990); Clostridium cellulolyticum (ref|ZP_{—}01574143); and Anaerotruncus colihominis (ref|ZP_{—}02441436), each of which is incorporated herein by reference. In certain aspects and AdsA polypeptide can have at least or more than 40%, 50%, 60%, 70%, 80%, 85%, 90%, 95%, 99%, identity, including all values and ranges there between, to SEQ ID NO:36 or SEQ ID NO:41.

In a further embodiment of the invention a composition may include a polypeptide, peptide, or protein that is or is at least 70%, 75%, 80%, 85%, 90%, 95%, 96%, 97%, 98%, or 99% identical or similar to an AdsA polypeptide (SEQ ID NO:36) or AdsA nucleic acid (SEQ ID NO:35), in certain aspects the AdsA polypeptide will have an amino acid sequence of (SEQ ID NO:36). Similarity or identity, with identity being preferred, is known in the art and a number of different programs can be used to identify whether a protein (or nucleic acid) has sequence identity or similarity to a known sequence. Sequence identity and/or similarity is determined using standard techniques known in the art, including, but not limited to, the local sequence identity algorithm of Smith & Waterman (1981), by the sequence identity alignment algorithm of Needleman & Wunsch (1970), by the search for similarity method of Pearson & Lipman (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (1984), preferably using the default settings, or by inspection. Preferably, percent identity is calculated by using alignment tools known to and readily ascertainable to those of skill in the art.

The compositions may be formulated in a pharmaceutically acceptable composition. In certain aspects of the invention the staphylococcus bacterium is an S. aureus or S. epidermidis bacterium. In another aspect, the bacteria is a bacillus or B. anthracis.

The activity of the compounds as inhibitors of AdsA can be assessed using methods known to those of skill in the art, as well as methods described herein. Screening assays may include controls for purposes of calibration and confirmation of proper manipulation of the components of the assay. Blank wells that contain all of the reactants but no member of the chemical library are usually included. As another example, a known inhibitor (or activator) of AdsA, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. The term “high throughput screening” or “HTS” as used herein refers to the testing of many thousands of molecules (or test compounds) for their effects on the function of a protein. In the case of group transfer reaction enzymes many molecules may be tested for effects on their catalytic activity. HTS methods are known in the art and they are generally performed in multiwell plates with automated liquid handling and detection equipment; however, it is envisioned that the methods of the invention may be practiced on a microarray or in a microfluidic system. The term “library” or “drug library” as used herein refers to a plurality of chemical molecules (test compounds) having potential as a modulator of AdsA, a plurality of nucleic acids, a plurality of peptides, or a plurality of proteins, and a combination thereof. Wherein the screening is performed by a high-throughput screening technique, wherein the technique utilizes a multiwell plate or a microfluidic system.

One example of an assay/kit for assessing AdsA activity includes, but is not limited to a Diazyme Enzyme reaction kit: This kit is a 5′-Nucleotidase (5′-NT) assay kit is typically used for the determination of 5′-NT activity in human serum samples. The 5′-NT assay is based on the enzymatic hydrolysis of 5′-monophosphate (5′-IMP) to form inosine which is converted to hypoxanthine by purine nucleoside phosphorylase (PNP). Hypoxanthine is then converted to uric acid and hydrogen peroxide (H₂O₂) by xanthine oxidase (XOD). H₂O₂ is further reacted with N-Ethyl-N-(2-hydroxy-3-sulfopropyl)-3-methylaniline (EHSPT) and 4-aminoantipyrine (4-AA) in the presence of peroxidase (POD) to generate quinone dye which is monitored kinetically. This method is fast, but is not as sensitive as the radioactivity assays.

Inhibitors and inhibitor candidates include, but are not limited to derivatives or analogs of: α,β-methylene adenosine 5′-diphosphate (AOPCP), an inhibitor of 5′-ecto nucleotidase (human homologue of bacterial AdsA), this inhibitor does not inhibit secreted 5′-nucleotidases from trophozoites of Trichomonas gallinae (Borges et al., 2007); nucleoticidin and melanocidins A and B, these compounds exhibited potent inhibitory activity against 5′-nucleotidases from rat liver membrane and snake venom (Uchino et al., 1986); polyphenolic compounds, these compounds poss-sess anti-tumor activity and inhibit 5′-nucleotidases from a variety of sources and have been isolated from the seeds of Areca catechu (betel nuts) as well as grapes (Iwamoto et al., 1988; Uchino et al., 1988; Toukairin et al., 1991).

In still further embodiments, a composition comprises a recombinant nucleic acid molecule encoding an AdsA polypeptide or segments/fragments thereof. Typically a recombinant nucleic acid molecule encoding an AdsA polypeptide contains a heterologous promoter.

In certain aspects, a recombinant nucleic acid molecule of the invention is a vector, in still other aspects the vector is a plasmid. In certain embodiments the vector is a viral vector. Aspects of the invention include compositions that further comprise a nucleic acid encoding an additional 1, 2, 3, 4, 5, 6, 7, 8, or more polypeptide or peptide.

In certain aspects a composition includes a recombinant, non-staphylococcus bacterium containing or expressing one or more polypeptide described herein in, e.g., an AdsA polypeptide. In particular aspects the recombinant non-staphylococcus bacteria is Salmonella or another gram-positive bacteria. A composition is typically administered to mammals, such as human subjects, but administration to other animals capable of eliciting an immune response is contemplated. In further aspects the staphylococcus bacterium containing or expressing the AdsA polypeptide is a Staphylococcus aureus. In further embodiments the immune response is a protective and/or therapeutic immune response.

Still further embodiments include methods for stimulating in a subject a protective or therapeutic immune response against a staphylococcus bacterium comprising administering to the subject an effective amount of a composition including (i) an AdsA polypeptide or peptide thereof; or, (ii) a nucleic acid molecule encoding an AdsA polypeptide or peptide thereof, or (iii) administering an AdsA polypeptide with any combination or permutation of bacterial proteins or polysaccharides described herein.

Yet still further embodiments include vaccines comprising a pharmaceutically acceptable composition having an isolated AdsA polypeptide, a segment or fragment thereof, or any other combination or permutation of protein(s) or peptide(s) described, wherein the composition is capable of stimulating an immune response against a staphylococcus bacterium. The vaccine may comprise an isolated AdsA polypeptide and/or any other combination or permutation of protein(s), peptide(s) or polysaccharides described. In certain aspects of the invention the isolated AdsA polypeptide, or any other combination or permutation of protein(s) or peptide(s) described are multimerized, e.g., dimerized.

In a further aspect, the vaccine composition is contaminated by less than about 10, 9, 8, 7, 6, 5, 4, 3, 2, 1, 0.5, 0.25, 0.05% (or any range derivable therein) of other Staphylococcal proteins. A composition may further comprise an isolated non-AdsA polypeptide. Typically the vaccine comprises an adjuvant. In certain aspects a protein or peptide of the invention is linked (covalently or non-covalently coupled) to the adjuvant, preferably the adjuvant is chemically conjugated to the protein.

In still yet further embodiments, a vaccine composition is a pharmaceutically acceptable composition having a recombinant nucleic acid encoding all or part of an AdsA polypeptide, and/or any other combination or permutation of protein(s) or peptide(s) described herein, wherein the composition is capable of stimulating an immune response against a staphylococcus or bacillus bacteria. The vaccine composition may comprise a recombinant nucleic acid encoding all or part of an AdsA polypeptide, and/or any other combination or permutation of protein(s) or peptide(s) described. In certain embodiments the recombinant nucleic acid contains a heterologous promoter. Preferably the recombinant nucleic acid is a vector. More preferably the vector is a plasmid or a viral vector.

In further embodiments, a vaccine composition is a pharmaceutically acceptable composition comprising an isolated antibody, or fragment thereof, that binds an AdsA protein or antigen, or a fragment thereof, wherein the composition is capable of attenuating a staphylococcus bacterial infection in a subject, e.g., modulating phagocytic uptake of bacteria. The antibody can be a human or humanized antibody. In certain aspects the antibody is a polyclonal antibody, or monoclonal antibody, or single chain antibody, or fragment thereof.

The vaccine composition can further comprise at least one additional isolated antibody that binds a antigen selected from a group consisting of an isolated ClfA, ClfB, EsaB, EsaC, EsxA, EsxB, Hla, IsdA, IsdB, IsdC, Emp, Eap, SasB, SasF, SdrC, SdrD, Coa, Ebh, vWa, SdrE, and SpA antigen, or a fragment thereof.

Still further embodiments include methods for stimulating in a subject a protective or therapeutic immune response against a staphylococcus bacterium comprising administering to the subject an effective amount of a composition of an AdsA polypeptide or segment/fragment thereof comprising one or more of (i) a Eap, Emp, EsaB, EsaC, EsxA, EsxB, SdrC, SdrD, SdrE, Hla or a variant thereof, IsdA, IsdB, IsdC, Spa, ClfA, ClfB, Coa, Ebh, vWa, IsdC, SasB, SasF, or SpA polypeptide or segment or fragment thereof; or, (ii) a nucleic acid molecule encoding the same. Methods of the invention also include AdsA compositions that contain 1, 2, 3, 4, 5, 6, 7, 8, 9, 10 or more of Eap, Emp, EsaB, EsaC, EsxA, EsxB, SdrC, SdrD, Coa, Ebh, vWa, SdrE, Hla or a variant thereof, IsdA, IsdB, IsdC, SpA, ClfA, ClfB, IsdC, SasB, SasF, or Spa in various combinations. In certain aspects a vaccine formulation includes an IsdA polypeptide or segment or fragment thereof.

In still a further aspect the invention includes a staphylococcal bacterium lacking an AdsA polypeptide. Such a bacterium will be limited or attenuated with respect to its ability to evade phagocyte uptake and/or recognition. This characteristic can be used to provide bacterial strain for the production of attenuated bacteria for use in the preparation of vaccines or treatments for staphylococcal infections or related diseases. In yet a further aspect, AdsA can be overexpressed in an attenuated bacterium to further enhance or supplement an immune response or vaccine formulation.

Any embodiment discussed with respect to one aspect of the invention applies to other aspects of the invention as well. In particular, any embodiment discussed in the context of an AdsA peptide or nucleic acid may be implemented with respect to other secreted virulence factors, and/or cell surface proteins, such as Eap, Emp, EsaB, EsaC, Coa, Ebh, vWa, EsxA, EsxB, SdrC, SdrD, SdrE, Hla or a variant thereof, IsdA, IsdB, ClfA, ClfB, IsdC, SasB, SasF, or SpA proteins or nucleic acids or antibodies that bind the same, and vice versa.

The term “providing” or “administering” is used according to its ordinary meaning to indicate “to supply or furnish for use.” In some embodiments, the protein is provided directly by administering the protein, while in other embodiments, the protein is effectively provided or administered by administering a nucleic acid that encodes the protein. In certain aspects the invention contemplates compositions comprising various combinations of antibodies, nucleic acid, antigens, peptides, epitopes, and/or polysaccharides and the like.

The subject typically will have (e.g., diagnosed with a persistent staphylococcal infection), will be suspected of having, or will be at risk of developing a staphylococcal infection. Compositions of the present invention include immunogenic compositions wherein the antigen(s) or epitope(s) are contained in an amount effective to achieve the intended purpose (e.g., treating or preventing infection). More specifically, an effective amount means an amount of active ingredients necessary to stimulate or elicit an immune response, or provide resistance to, amelioration of, or mitigation of infection. In more specific aspects, an effective amount prevents, alleviates, or ameliorates symptoms of disease or infection, or prolongs the survival of the subject being treated. Determination of the effective amount is well within the capability of those skilled in the art, especially in light of the detailed disclosure provided herein. For any preparation used in the methods of the invention, an effective amount or dose can be estimated initially from in vitro, cell culture, and/or animal model assays. For example, a dose can be formulated in animal models to achieve a desired immune response or circulating antibody concentration or titer. Such information can be used to more accurately determine useful doses in humans.

As used herein, the term “modulate” or “modulation” encompasses the meanings of the words “enhance,” or “inhibit.” “Modulation” of activity may be either an increase or a decrease in activity. As used herein, the term “modulator” refers to compounds that effect the function of a moiety, including up-regulation, induction, stimulation, potentiation, inhibition, down-regulation, or suppression of a protein, nucleic acid, gene, organism or the like.

The term “isolated” can refer to a nucleic acid or polypeptide or peptide that is substantially free of cellular material, bacterial material, viral material, or culture medium (when produced by recombinant DNA techniques or isolated from naturally occurring organism(s)) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that does not naturally occur and/or function as a fragment and/or is not typically in the functional state.

Moieties of the invention, such as antibodies, polypeptides, peptides, antigens or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.” Recombinant fusion proteins are particularly contemplated. Compositions of the invention may further comprise an adjuvant or a pharmaceutically acceptable excipient. An adjuvant may be covalently or non-covalently coupled to a polypeptide or peptide of the invention. In certain aspects, the adjuvant is chemically conjugated to a protein, polypeptide, or peptide. In still a further aspect the adjuvant is part of a recombinant protein and is comprised in a fusion protein comprising one or more antigens of interest.

The compositions may be formulated in a pharmaceutically acceptable composition. In certain aspects of the invention the staphylococcus bacterium is an S. aureus bacterium.

In further aspects of the invention a composition may be administered more than one time to the subject, and may be administered 1, 2, 3, 4, 5, 6, 7, 8, 9, 10, 15, 20 or more times. The administration of the compositions include, but is not limited to nasal, pleural, oral, parenteral, subcutaneous, intramuscular, intravenous administration, or various combinations thereof, including inhalation or aspiration.

In still further embodiments, a composition comprises a recombinant nucleic acid molecule encoding an Emp, Eap and/or AdsA polypeptide or segments/fragments thereof. Typically a recombinant nucleic acid molecule encoding an Emp, Eap and/or AdsA polypeptide contains a heterologous promoter.

In certain aspects, a recombinant nucleic acid molecule of the invention is a vector, in still other aspects the vector is a plasmid. In certain embodiments the vector is a viral vector. Aspects of the invention include compositions that further comprise a nucleic acid encoding an additional 1, 2, 3, 4, 5, 6, 7, 8, or more polypeptides or peptides.

In certain aspects a composition includes a recombinant, non-staphylococcus bacterium containing or expressing one or more polypeptides described herein in, e.g., an Emp Eap and/or AdsA polypeptide. In particular aspects the recombinant non-staphylococcus bacteria is Salmonella or another gram-positive bacteria. A composition is typically administered to mammals, such as human subjects, but administration to other animals capable of eliciting an immune response is contemplated. In further aspects the staphylococcus bacterium containing or expressing the Emp, Eap and/or AdsA polypeptide is a Staphylococcus aureus. In further embodiments the immune response is a protective immune response.

Compositions of the invention are typically administered to human subjects, but administration to other animals that are capable of eliciting an immune response to a staphylococcus bacterium is contemplated, particularly mice, dogs, cats, cattle, horses, goats, sheep and other domestic animals, i.e., mammals, including transgenic animals (e.g., animal manipulated to express human antibodies).

In still further aspects, the methods and compositions of the invention can be used to prevent, ameliorate, reduce, or treat infection of tissues or glands, e.g., mammary glands, particularly mastitis and other infections. Other methods include, but are not limited to prophylatically reducing bacterial burden in a subject not exhibiting signs of infection, particularly those subjects suspected of or at risk of being colonized by a target bacteria, e.g., patients that are or will be at risk or susceptible to infection during a hospital stay, treatment, and/or recovery.

Any list provided herein may specifically exclude or include any 1, 2, 3, 4, 5, 6, 7, 8, 9, 10 or more members of the list.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate certain embodiments of the invention and therefore are not to be considered limiting in their scope.

FIGS. 1A-1H Examination of abscess formation in staphylococcus aureus. (FIG. 1A) Photograph of Newman infected kidneys. (FIG. 1B) Photograph of srtA mutant infected kidneys. (FIG. 1C) H&E histological section of Newman infected kidney. (FIG. 1D) Histological section of ΔSrtA infected kidney. (FIG. 1E) Closeup of Newman infected kidney. (FIG. 1F) Closeup of ΔSrtA infected kidney. (FIG. 1G) Scanning Electron Microscopy of Newman abscess. (FIG. 1H) SEM of ΔSrtA infected kidney.

FIG. 2 Murine renal abscess screen. Recovered colony forming units (CFU) from kidneys infected with respective mutant strains.

FIGS. 3A-3G Biofilm screen. (FIG. 3A) 96 well plate assay for in vitro biofilm growth. (FIG. 3B) SEM Newman in vitro biofilm (FIG. 3C) ΔsrtA biofilm. (FIG. 3D) ΔEmp biofilm. (FIG. 3E) ΔIcaA biofilm. (FIG. 3F) ΔIsdB biofilm. (FIG. 3G) ΔSdrD biofilm.

FIGS. 4A-4B Emp virulence. (FIG. 4A) Recovered CFUs for Newman, ΔEap, ΔEmp, ΔSrtA, ΔEap/ΔSrtA, ΔEmp/ΔSrtA. (FIG. 4B) Histopathology for respective strains.

FIGS. 5A-5E Eap, Emp vaccination. (FIG. 5A) SDS extraction of Newman, ΔSrtA, ΔIsdB, ΔIcaA, ΔEap, ΔEmp, ΔSaeR. (FIG. 5B) Protein purification of Eap (70 kDa) and Emp (45 kDa). (FIG. 5C) Recovered CFUs from mice vaccinated with PBS, Eap, or Emp and challenged with Newman. (FIG. 5D) ELISA IgG titers from vaccinated mice. (FIG. 5E) Histopathology from vaccinated mice.

FIGS. 6A-6B Ica virulence. (FIG. 6A) Recovered CFUs from mice infected with Newman, DIcaA, DIcaB, DIcaC, DIcaD, DIcaR, DIca:tet (entire operon deletion). (FIG. 6B) Histopathology from infected mice.

FIGS. 7A-7K Staphylococcal abscess formation following intravenous infection of mice. (A) BALB/c mice were infected with 1×10₇ colony forming units (CFU) of S. aureus Newman by retro-orbital injection. Cohorts of five mice were examined by cardiac puncture at timed intervals for bacterial load in blood; sample aliquots were plated on agar medium and CFU per ml of blood were enumerated. The means of these observations is indicated by a black bar. (B) Dissemination of S. aureus Newman into peripheral organ tissues and replication of the pathogen was measured at timed intervals in the kidneys of mice (cohorts of ten animals), which were homogenized and plated on agar medium for CFU. (C) Diameter of abscess lesions were measured in thin-sectioned hematoxylin-eosin stained tissues of infected kidneys at timed intervals. (D-K) Images of infected kidneys at timed intervals analyzed in thin-sectioned hematoxylin-eosin stained tissues. Arrowheads point to abscess lesions.

FIGS. 8A-8F Histopathology of staphylococcal abscess communities. BALB/c mice were infected with S. aureus Newman via retro-orbital injection. Thin-sectioned, hematoxylineosin stained tissues of infected kidneys on day 2 (ABC) and day 5 following infection (DEF) were analyzed by light microscopy and images captured. On day 2, a massive infiltrate (blue arrow in A) of polymorphonuclear granulocytes (PMNs) with occasional intracellular staphylococci (yellow arrows in C) are characteristic of early infectious lesions. By day 5, staphylococcal abscess communities developed as a central nidus (D, black arrow). Staphylococci were enclosed by an amorphous, eosinophilic pseudocapsule (boxed in black) and surrounded by a zone of dead PMNs (boxed in white), a zone of apparently healthy PMNs (boxed in red) and a rim of necrotic PMNs (boxed in green), separated through an eosinophilic layer from healthy kidney tissue.

FIGS. 9A-9P Sortase A is required for abscess formation and staphylococcal persistence in host tissues. Kidneys of BALB/c mice (cohorts of ten animals) infected with S. aureus Newman, its isogenic sortase A mutant (ΔsrtA) or methicillin-resistant S. aureus USA300 were removed during necropsy of animals 5 (d5) and 15 days (d15) following inoculation. Kidneys were inspected for surface abscesses (A, F, K) or fixed in formalin, embedded, thin sectioned and stained with hematoxylin-eosin. Histopathology images were acquired with light microscopy at 10×(B, G, L, D, I, N) and 100×fold magnification (C, H, M, E, J, O). (P) Staphylococcal replication and persistence in kidney tissue was measured 5 and 15 days following infection. Kidneys were removed from infected mice during necropsy, tissue was homogenized and plated on agar medium for colony formation and enumeration.

FIGS. 10A-10C Staphylococcal communities at the center of abscess lesions. Kidney tissue from mice infected with S. aureus Newman (wild-type), its isogenic sortase A mutant (ΔsrtA), or MRSA strain USA300 was sectioned, fixed, dehydrated and sputter coated with 80% platinum/20% palladium for scanning electron microscopy. (A) The wild-type pathogen is organized as a tightly associated lawn, the staphylococcal abscess community (SAC), at the abscess center that is contained within an amorphous pseudocapsule (white arrow heads), separating SACs from the cuff of leukocytes. Red blood cells were located among staphylococci (R). (B) The sortase mutant (ΔsrtA, arrows) did not form SACs and isolated staphylococci were found in healthy kidney tissue. (C) Similar to S. aureus Newman, MRSA strain USA300 also formed SACs contained within a pseudocapsule (white arrow heads).

FIGS. 11A-11B Formation of staphylococcal abscess communities requires specific surface proteins. (A) S. aureus Newman variants with bursa aurealis insertions in surface protein genes were examined five days following infection of BALB/c mice (cohorts of 20 animals) for bacterial load in homogenized kidney tissues. (B) Hematoxylin-eosin stained thin sections of infected kidneys were examined by light microscopy and 10× fold magnification for abscess lesions (white arrows).

FIGS. 12A-12L Emp and Eap in staphylococcal abscess lesions. Kidneys of BALB/c mice infected with S. aureus Newman variants carrying bursa aurealis insertions in emp or eap were removed 5 (d5) and 15 days (d15) following inoculation. Kidneys were stained with hematoxylin-eosin and histopathology images acquired with light microscopy at 10×(A, C, E, H) and 100× fold magnification (B, D, F, I). Expression of Eap (J) and Emp (K) in abscess lesions of wild-type S. aureus Newman were detected with rabbit anti-Emp or anti-Eap and secondary Alexafluor-647 labeled antibodies (red) in renal tissue stained with Hoechst-dye (blue) to detect nuclei of polymorphonuclear leukocytes, and with BODIPY-vancomycin (green) to reveal staphylococcal abscess communities. (L) Staphylococcal replication and persistence in kidney tissue was measured 5, 15 and 30 days following intravenous inoculation. Kidneys were removed from infected mice (cohorts of 10 animals), tissue was homogenized and plated on agar medium for colony formation and enumeration.

FIGS. 13A-13E Active and passive immunization with Eap generates protection from staphylococcal challenge. (A) BALB/c mice were immunized with purified Eap or Emp or mock treated with adjuvant alone and serum IgG titers analyzed by ELISA. (B) Three weeks following immunization, animals were challenged via intravenous inoculation of staphylococci. Five days following infection, kidneys were removed during necropsy and renal tissue analyzed for staphylococcal load or histopathology. (C) Rabbit antibodies directed against Eap or Emp were purified by affinity chromatography and passively transferred by intraperitoneal injection into mice. Twenty-four hours later, serum IgG titers of passively immunized animals were analyzed by ELISA. (D) Animals passively immunized with purified antibodies against Eap or Emp as well as mock immunized animals subsequently challenged with S. aureus Newman and bacterial load enumerated on day 4. (E) Abscess formation in kidneys was detected in thin-sectioned, hematoxylin-eosin stained tissues.

FIG. 14 A working model for staphylococcal abscess formation and persistence in host tissues. Stage I—following intravenous inoculation, S. aureus survives in the blood stream and disseminates via the vasculature to peripheral organ tissues. Stage II—in renal tissues, staphylococci attract a massive infiltrate of polymorphonuclear leukocytes and other immune cells. Stage III—abscesses mature with a central accumulation of the pathogen (staphylococcal abscess communities—SAC), enclosed by an eosinophilic pseudocapsule. The SAC is surrounded by a zone of dead PMNs, apparently healthy PMNs and finally an outer zone of dead PMNs with a rim of eosinophilic material. Stage 4—abscesses mature and rupture on the organ surface, thereby releasing staphylococci into circulation and initiating new rounds of abscess development. Genes for bacterial envelope components that are required for specific stages of staphylococcal abscess development are printed in red underneath the corresponding stage during which these genes function.

FIGS. 15A-15H AdsA is a cell wall associated protein essential for survival in blood. Comparison of the survival of wild-type S. aureus Newman (WT) and isogenic srtA variants in blood from BALB/c mice (FIG. 15A) or Sprague-Dawley rats (FIG. 15D). Data are the means and standard error of the means from three independent analyses (±SEM). To assess the relative contribution of sortase A-anchored cell wall surface proteins for staphylococci survival in blood, isogenic mutants with transposon insertions in the indicated genes were incubated in blood from mice (FIG. 15B) or rats (FIG. 15E) for 60 minutes. Expression of padsA rescues staphylococcal survival of an adsA mutant in blood from mice (FIG. 15C), rats (FIG. 15F), or human volunteers (FIG. 15G). Visualization of WT, adsA, and adsA (padsA) staphylococci with phagocytic cells in Giemsa-stained human blood samples (FIG. 15H). Arrows indicate both extracellular and neutrophil associated S. aureus. Data are representative of two independent analyses with two different donors.

FIGS. 16A-16E AdsA is a virulence factor that enables staphylococcal replication and abscess formation in vivo. Staphylococcal burden in kidneys after infection of cohorts of 10 BALB/c mice with S. aureus Newman wild-type and adsA mutant (FIG. 16A) or USA300 wild-type and adsA mutant (FIG. 16C) (P<0.03 for infections for both Newman and USA300, unpaired t-test). Microscopic images of hematoxylin-eosin stained kidney tissue at ×10 (top panels) and ×100 magnification (lower panels) obtained following necropsy of mice infected with S. aureus Newman wild-type and adsA mutant (FIG. 16B) or S. aureus USA300 wild-type and adsA mutant (FIG. 16D). Black arrows denote a central concentration of staphylococci and PMN infiltrates. Data are representative samples of cohorts of 5 animals per bacterial strain and 2 independent analyses. (FIG. 16E) Bacterial load was measured as CFUs per 500 μl blood obtained from BALB/c mice infected by retroorbital injection with either wild-type (WT), adsA or adsA:padsA S. aureus Newman for 30 or 90 minutes. Data are representative of two independent analyses using cohorts of 10 animals for each time point. Unpaired t-test was used for statistical analysis.

FIGS. 17A-17F AdsA exhibits 5′-nucleotidase activity and hydrolyzes AMP. Lysostaphin cell wall extracts from the indicated bacterial strains were incubated with radiolabeled [¹⁴C]AMP and generation of [¹⁴C]Ado (adenosine) was measured by thin layer chromatography (TLC). (FIG. 17A) Radioactive signals for [¹⁴C]AMP and [¹⁴C]Ado following TLC were captured by PhosphorImager. (FIG. 17B) Radioactive [¹⁴C]Ado signals from (a) were quantified, calibrated for adenosine synthase activity in S. aureus Newman (100%) and displayed as percent amount. Data are the means of three independent analyses, error bars represent SEM. (P<0.05 for WT vs. adsA). (FIG. 17C) Radiolabeled [¹⁴C]AMP was incubated in the presence or absence of purified AdsA₁₋₄₀₀ (2 μM) in the presence or absence of 5 mM of various metal ions. Radioactive signals for [¹⁴C]AMP and [¹⁴C]Ado following TLC were captured by PhosphorImager. (FIG. 17D) Radioactive [¹⁴C]Ado signals from (c) were quantified, calibrated for adenosine synthase activity in the presence of manganese chloride (Mn²⁺)(100%) and displayed as percent amount. Displayed data are the mean of 2 independent analyses and error bars represent SEM. (P<0.05 for Zn⁺² vs Mn⁺² and Cu⁺² vs Mn⁺²). (FIG. 17E) GST-AdsA was purified from recombinant Escherichia coli, cleaved with thrombin to generate AdsA₁₋₄₀₀ and purified proteins were analyzed by Coomassie-stained SDS-PAGE. (FIG. 17F) Survival of adsA staphylococci in rat blood in the presence or absence of variable concentrations of adenosine.

FIGS. 18A-18D Staphylococcal AdsA synthesizes adenosine in blood. (FIG. 18A) Reversed-phase high performance liquid chromatography (RP-HPLC) to quantify adenosine (left panel, 100 μM adenosine) and identify its monoisotopic ions by matrix assisted laser desorption ionization mass spectrometry (MALDI-MS, right panel). Mouse blood was incubated without (FIG. 18B) or with S. aureus Newman wild-type (WT) (FIG. 18C) or its isogenic adsA variants (FIG. 18D) for one hour. Plasma was deproteinized, filtered and subjected to RP-HPLC to quantify adenosine (left panels) and identify its monoisotopic ions by MALDI-MS (right panels). Calculated abundance of adenosine in blood extrapolated from the purified adenosine control was 1.1 μM (18B, no staphylococci), 13.2 μM (18C, WT S. aureus Newman) and 2.1 μM (18D, adsA mutant staphylococci).

FIGS. 19A-19E 5′-Nucleotidase activity enhances B. anthracis survival. (FIG. 19A) Mutanolysin extracts from B. anthracis strain Sterne (WT, wild-type) or adsA (basA) mutant bacilli were incubated with radiolabed [¹⁴C]AMP and generation of adenosine was measured by TLC. (FIG. 19B) Proteins from mutanolysin extracts were analyzed with antisera raised against BasA (aBasA) or BasC (aBasC), a control protein not involved in adenosine production. (FIG. 19C) Fluorescence microsocopy images of wild-type (WT) B. anthracis Sterne and its isogenic adsA mutant stained with antiserum against BasA (top panel) or non-reactive serum (NRS) and Cy3-labeled secondary antibodies (red) as well as Hoechst staining of nucleic acids (blue). (FIG. 19D) Radiolabeled [¹⁴C]AMP was incubated with purified BasA (2 μM) in the presence of 5 mM of variable metal cations and generation of [¹⁴C]Ado (adenosine) was measured by thin layer chromatography (TLC) and PhosphorImager. Data are representative of 3 independent analyses. (FIG. 19E) Survival of wild-type and adsA/basA mutant B. anthracis strain Sterne in rat blood over time (minutes) measured as colony forming units on agar plates. Data are the average of two independent analyses and error bars represent the SEM.

FIG. 20 Visualization of adsA disruption and padsA complementation. To allow visualization of AdsA, we used a Protein A deficient (Aspa) S. aureus strain SEJ2, as Protein A specifically binds to Fc domains of antibodies and interferes with immunoblotting analyses. Cell wall extracts from wild type Aspa SEJ2 (lane 1), Δspa, adsA:ermB (lane 2) or Δspa, adsA:ermB cells transformed with padsA were separated by SDS-PAGE and immunoblotting analyses conducted with anti-sera raised against GST-AdsA₁₋₄₀₀. * denotes non-specific reactive species

FIG. 21 Histological examination of kidneys isolated from mice infected with USA300. Microscopic images of hematoxylin-eosin stained kidney tissue at x10 obtained following necropsy of mice infected for 4 days with S. aureus USA300 wild-type (bottom panels) and adsA mutants (top panels). Black arrows denote a central concentration of staphylococci and PMN infiltrates. Data are representative samples of cohorts of 5 animals per bacterial strain and 2 independent analyses.

### DETAILED DESCRIPTION OF THE INVENTION

Biofilms are microbial communities embedded in a secreted extracellular matrix (Hall-Stoodley et al., 2004; Kolter and Greenberg, 2006). Many bacterial species are capable of switching from planktonic growth to the formation of biofilms and thereby display increased antibiotic resistance (Drenkard and Ausubel, 2002), evasion from host immune defenses (Singh et al., 2002), and are more adept at establishing chronic infections in humans (Brady et al., 2008). Biofilms of staphylococcal species have been associated with a number of diseases including endocarditis (Xiong et al., 2005), osteomyelitis (Brady et al., 2006), and various implant-mediated infections including urinary catheters, prosthetic heart valves, and artificial joints (Cassat et al., 2007). This applies in particular to Staphylococcus epidermidis, an opportunistic pathogen that avidly forms biofilms in vitro and in vivo (Mack et al., 1996). While there is clear association between the ability to form biofilms and virulence in S. epidermidis, such correlation has not yet been demonstrated for S. aureus (Cassat et al., 2007).

S. aureus is a commensal of human skin and nares and the leading cause of bloodstream and skin/soft tissue infections (Klevens et al., 2007). The pathogenesis of staphylococcal infections is initiated as bacteria invade skin or blood stream via trauma, surgical wounds, or medical devices (Lowy, 1998). Some staphylococci are cleared from the blood stream by phagocytic killing, however staphylococci that escape immune defenses seed infections in organ tissues and induce a proinflammatory response mediated by the release of cytokines and chemokines from macrophages, neutrophils, and other phagocytes (Lowy, 1998). The resulting invasion of immune cells to the site of infection is accompanied by central liquefaction necrosis and formation of peripheral fibrin walls in an effort to prevent microbial spread and allow for removal of necrotic tissue debris (Lowy, 1998). Such lesions can be observed by microscopy as hypercellular areas containing necrotic tissue, leukocytes, and a central nidus of bacteria. Organ abscesses occur within two days of infection (unpublished data) and represent a hallmark of staphylococcal disease.

### I. Staphylococcal Antigens

The Staphylococcus aureus Ess pathway can be viewed as a secretion module equipped with specialized transport components (Ess), accessory factors (Esa), and cognate secretion substrates (Esx). EssA, EssB and EssC are required for EsxA and EsxB secretion. Because EssA, EssB and EssC are predicted to be transmembrane proteins, it is contemplated that these proteins form a secretion apparatus. Some of the proteins in the ess gene cluster may actively transport secreted substrates (acting as motor) while others may regulate transport (regulator). Regulation may be achieved, but need not be limited to, transcriptional or post-translational mechanisms for secreted polypeptides, sorting of specific substrates to defined locations (e.g., extracellular medium or host cells), or timing of secretion events during infection. At this point, it is unclear whether all secreted Esx proteins function as toxins or contribute indirectly to pathogenesis.

Staphylococci rely on surface protein mediated-adhesion to host cells or invasion of tissues as a strategy for escape from immune defenses. Furthermore, S. aureus utilize surface proteins to sequester iron from the host during infection. The majority of surface proteins involved in staphylococcal pathogenesis carry C-terminal sorting signals, i.e., they are covalently linked to the cell wall envelope by sortase. Further, staphylococcal strains lacking the genes required for surface protein anchoring, i.e., sortase A and B, display a dramatic defect in the virulence in several different mouse models of disease. Thus, surface protein antigens represent a validated vaccine target as the corresponding genes are essential for the development of staphylococcal disease and can be exploited in various embodiments of the invention. The sortase enzyme superfamily are Gram-positive transpeptidases responsible for anchoring surface protein virulence factors to the peptidoglycan cell wall layer. Two sortase isoforms have been identified in Staphylococcus aureus, SrtA and SrtB. These enzymes have been shown to recognize a LPXTG motif in substrate proteins. The SrtB isoform appears to be important in heme iron acquisition and iron homeostasis, whereas the SrtA isoform plays a critical role in the pathogenesis of Gram-positive bacteria by modulating the ability of the bacterium to adhere to host tissue via the covalent anchoring of adhesions and other proteins to the cell wall peptidoglycan.

Embodiments of the invention include, but are not limited to compositions and methods related to Emp and/or Eap. In certain embodiment Emp and/or Eap can be used in combination with other staphylococcal proteins such as EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, and/or SpA proteins. Emp (SEQ ID NO:2) or Eap (SEQ ID NO:4) are staphylococcal polypeptides. Sequence of other Emp and/or Eap polypeptides can be found in the protein databases and include, but are not limited to accession numbers YP_{—}185731, NP_{—}371337, NP_{—}645584, CAB75985, YP_{—}416239, YP_{—}040269, and NM0758 for Emp and YP_{—}500650, CAB94853, YP_{—}186825, CAB51807, NP_{—}646697, YP_{—}041404, NM1872 for Eap, each of which is incorporated herein by reference as of the priority date of this application. Additional Staphyloccal antigens include 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein.

In mammals, adenosine assumes an essential role in regulating innate and acquired immune responses (Thiel et al., 2003). Strong or excessive host inflammatory responses, for example in response to bacterial infection, exacerbate the tissue damage inflicted by invading pathogens (Thiel et al., 2003). Successful immune clearance of microbes therefore involves the balancing of pro- and anti-inflammatory mediators. Cytokines IL-4, IL-10, IL-13 and TGF-β restrict excessive inflammation, however only adenosine is able to completely suppress immune responses (Nemeth et al., 2006). The immunoregulatory attributes of adenosine are mediated via four transmembrane adenosine receptors: A₁, A_{2A}, A_{2B}, and A₃ (Hasko and Pacher, 2008). T lymphocytes express the high affinity A_{2A} receptor as well as the low affinity A_{2B} receptor (Thiel et al., 2003). Depending on their activation state, macrophages and neutrophils express all four adenosine receptors, whereas B cells harbor only A_{2A} (Thiel et al., 2003). Engagement of A_{2A} inhibits IL-12 production, increases IL-10 in monocytes (Khoa et al., 2001) and dendritic cells (Panther et al., 2001), and decreases cytotoxic attributes and chemokine production in neutrophils (McColl et al., 2006; Cronstein et al., 1986). Generation of adenosine at sites of inflammation, hypoxia, organ injury, and traumatic shock is mediated by two sequential enzymes. Ecto-ATP diphosphohydrolase (CD39) converts circulating adenosine triphosphate (ATP) and adenosine diphosphate (ADP) to 5′-adenosine monophosphate (AMP) (Eltzshig et al., 2003). CD73, expressed on the surface of endothelial cells (Deussen et al., 1993) and subsets of T cells (Thompson et al., 1989; Thompson et al., 1987; Yang et al., 2005), then converts 5′-AMP to adenosine (Zimmermann, 1992).

Although extracellular adenosine is essential for the suppression of inflammation, build-up of excess adenosine is also detrimental. This is exemplified in patients with a deficiency in adenosine deaminase (ADA), an enzyme that converts adenosine into inosine (Giblett, et al., 1972). ADA deficiency causes the severe compromised immunodeficiency syndrome (SCID) with impaired cellular immunity and severely decreased production of immunoglobulins (Buckley et al., 1997). As the regulation of extracellular adenosine is critical in maintaining immune homeostasis, perturbation of adenosine levels is likely to impact host immune responses during infection. The inventors describe herein that bacteria, e.g., S. aureus and Bacillus anthracis, use adenosine synthesis to escape host immune responses and provide methods and composition for utilizing this information for the treatment and/or prevention of bacterial infection, e.g., bacteremia.

Certain embodiments of the invention are directed to inducing an immune response, providing an antibody to, or inhibiting AdsA. AdsA (SEQ ID NO:55) is a staphylococcal polypeptide. Sequence of other AdsA polypeptides can be found in the protein databases and include, but are not limited: Staphlyococcus aureus (ref|YP_{—}001573948, ref|YP_{—}184935, ref|YP_{—}039500, ref|NP_{—}373261, ref|NP_{—}370547, ref|YP_{—}042156, ref|NP_{—}644838, ref|YP_{—}415541, dbj|BAA82250); Staphlyococcus hemolyticus (ref|YP_{—}254367); Streptococcus sanguinis (ref|YP_{—}001035187); Streptococcus gordonii (ref|YP_{—}001450531); Enterococcus faecalis (ref|NP_{—}813870); Streptococcus suis (dbj|BAB83980, ref|YP_{—}001200571, ref|YP_{—}001198366); Streptococcus mutans (ref|NP_{—}721592); Streptococcus thermophilus (ref|YP_{—}141373, ref|YP_{—}139455); Alkaliphilus metalliredigens (ref|YP_{—}001321391); Clostridium botulinum (ref|YP_{—}001887045, ref|YP_{—}001921966); Paenibacillus (ref|ZP_{—}02846642); Alkaliphilus oremlandii (ref|YP_{—}001512463); Bacillus clausii (ref|YP_{—}174466); Bacillus halodurans (ref|NP_{—}240892); Clostridium difficile (ref|ZP_{—}03126518, ref|ZP_{—}02748384, ref|YP_{—}001089051, ref|ZP 02726436, ref|ZP_{—}01801990); Clostridium cellulolyticum (ref|ZP_{—}01574143); Anaerotruncus colihominis (ref|ZP_{—}02441436), each of which is incorporated herein by reference as of the priority date of this application. In certain aspects, AdsA polypeptide can have at least or more than 40%, 50%, 60%, 70%, 80%, 85%, 90%, 95%, 99%, identity, including all values and ranges there between, to SEQ ID NO:36 or SEQ ID NO:41.

Certain aspects of the invention include methods and compositions concerning proteinaceous compositions including polypeptides, peptides, antibodies that bind such polypeptides and peptides, or nucleic acids encoding Emp and/or Eap and other staphylococcal antigens such as EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or combinations thereof. These proteins may be modified by deletion, insertion, and/or substitution.

Certain aspects of the invention include methods and compositions concerning proteinaceous compositions including polypeptides, peptides, and/or antibodies that bind such polypeptides and peptides, or nucleic acids encoding AdsA and other staphylococcal antigens such as Eap, Emp, EsaB, EsaC, EsxA, EsxB, SdrC, SdrD, SdrE, Hla or a variant thereof, IsdA, IsdB, ClfA, ClfB, IsdC, SasB, SasF, SasH (AdsA), Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or combinations thereof. These proteins may be modified by deletion, insertion, and/or substitution.

These polypeptides include the amino acid sequence of proteins from bacteria in the Staphylococcus genus. The sequence may be from a particular staphylococcus species, such as Staphylococcus aureus, and may be from a particular strain, such as Newman.

The sortase substrate polypeptides include, but are not limited to the amino acid sequence of SdrC, SdrD, SdrE, IsdA, IsdB, SpA, ClfA, ClfB, IsdC or SasF proteins from bacteria in the Staphylococcus genus. The sortase substrate polypeptide sequence may be from a particular staphylococcus species, such as Staphylococcus aureus, and may be from a particular strain, such as Newman. In certain embodiments, the SdrD sequence is from strain N315 and can be accessed using GenBank Accession Number NP_{—}373773.1 (gi|15926240), which is incorporated by reference. In other embodiments, the SdrE sequence is from strain N315 and can be accessed using GenBank Accession Number NP_{—}373774.1 (gi|15926241), which is incorporated by reference. In other embodiments, the IsdA sequence is SAV1130 from strain Mu50 (which is the same amino acid sequence for Newman) and can be accessed using Genbank Accession Number NP_{—}371654.1 (gi|15924120), which is incorporated by reference. In other embodiments, the IsdB sequence is SAV 1129 from strain Mu50 (which is the same amino acid sequence for Newman) and can be accessed using Genbank Accession Number NP_{—}371653.1 (gi|15924119), which is incorporated by reference. In further embodiments, other polypeptides transported by the Ess pathway or processed by sortase may be used, the sequences of which may be identified by one of skill in the art using databases and internet accessible resources.

Examples of various proteins that can be used in the context of the present invention can be identified by analysis of database submissions of bacterial genomes, including but not limited to accession numbers NC_{—}002951 (GI:57650036 and GenBank CP000046), NC_{—}002758 (GI:57634611 and GenBank BA000017), NC_{—}002745 (GI:29165615 and GenBank BA000018), NC_{—}003923 (GI:21281729 and GenBank BA000033), NC_{—}002952 (GI:49482253 and GenBank BX571856), NC_{—}002953 (GI:49484912 and GenBank BX571857), NC_{—}007793 (GI:87125858 and GenBank CP000255), NC_{—}007795 (GI:87201381 and GenBank CP000253) each of which are incorporated by reference.

As used herein, a “protein” or “polypeptide” refers to a molecule comprising at least ten amino acid residues. In some embodiments, a wild-type version of a protein or polypeptide are employed, however, in many embodiments of the invention, a modified protein or polypeptide is employed to generate an immune response. The terms described above may be used interchangeably. A “modified protein” or “modified polypeptide” refers to a protein or polypeptide whose chemical structure, particularly its amino acid sequence, is altered with respect to the wild-type protein or polypeptide. In some embodiments, a modified protein or polypeptide has at least one modified activity or function (recognizing that proteins or polypeptides may have multiple activities or functions). It is specifically contemplated that a modified protein or polypeptide may be altered with respect to one activity or function yet retain a wild-type activity or function in other respects, such as immunogenicity.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino molecules or greater, and any range derivable therein, or derivative of a corresponding amino sequence described or referenced herein. It is contemplated that polypeptides may be mutated by truncation, rendering them shorter than their corresponding wild-type form, but also they might be altered by fusing or conjugating a heterologous protein sequence with a particular function (e.g., for targeting or localization, for enhanced immunogenicity, for purification purposes, etc.).

As used herein, an “amino molecule” refers to any amino acid, amino acid derivative, or amino acid mimic known in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including (i) the expression of proteins, polypeptides, or peptides through standard molecular biological techniques, (ii) the isolation of proteinaceous compounds from natural sources, or (iii) the chemical synthesis of proteinaceous materials. The nucleotide as well as the protein, polypeptide, and peptide sequences for various genes have been previously disclosed, and may be found in the recognized computerized databases. One such database is the National Center for Biotechnology Information's Genbank and GenPept databases (on the World Wide Web at ncbi.nlm.nih.gov/). The coding regions for these genes may be amplified and/or expressed using the techniques disclosed herein or as would be known to those of ordinary skill in the art.

Amino acid sequence variants of Emp or Eap or AdsA and other polypeptides of the invention, EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein can be substitutional, insertional, or deletion variants. A modification in a polypeptide of the invention may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more non-contiguous or contiguous amino acids of the polypeptide, as compared to wild-type.

An antigen of the invention can comprise a segment or fragment of an antigen (AdsA, Emp, Eap, EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein) described herein comprising amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, or more (including all values and ranges there between) to amino acid 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, or more (including all values and ranges there between) of the antigen, including all segments and fragments there between. Certain aspects of the invention are directed to an antibody that binds one or more of the above described fragments or segments.

Deletion variants typically lack one or more residues of the native or wild-type protein. Individual residues can be deleted or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

TABLE 1
Exemplary surface proteins of S. aureus strains.

| SAV # | SA# | Surface | MW2 | Mu50 | N315 | Newman | MRSA252* | MSSA476* |
|---|---|---|---|---|---|---|---|---|
| SAV0111 | SA0107 | SpA | 492 | 450 | 450 | | 516 | 492 |
| SAV2503 | SA2291 | FnBPA | 1015 | 1038 | 1038 | | — | 1015 |
| SAV2502 | SA2290 | FnBPB | 943 | 961 | 961 | | 965 | 957 |
| SAV0811 | SA0742 | ClfA | 946 | 935 | 989 | 933 | 1029 | 928 |
| SAV2630 | SA2423 | ClfB | 907 | 877 | 877 | 913 | 873 | 905 |
| Np | np | Can | 1183 | — | — | | 1183 | 1183 |
| SAV0561 | SA0519 | SdrC | 955 | 953 | 953 | 947 | 906 | 957 |
| SAV0562 | SA0520 | SdrD | 1347 | 1385 | 1385 | 1315 | — | 1365 |
| SAV0563 | SA0521 | SdrE | 1141 | 1141 | 1141 | 1166 | 1137 | 1141 |
| Np | np | Pls | — | — | — | | — | — |
| SAV2654 | SA2447 | SasA | 2275 | 2271 | 2271 | | 1351 | 2275 |
| SAV2160 | SA1964 | SasB | 686 | 2481 | 2481 | | 2222 | 685 |
| | SA1577 | SasC | 2186 | 213 | 2186 | | 2189 | 2186 |
| SAV0134 | SA0129 | SasD | 241 | 241 | 241 | | 221 | 241 |
| SAV1130 | SA0977 | SasE/IsdA | 350 | 350 | 350 | | 354 | 350 |
| SAV2646 | SA2439 | SasF | 635 | 635 | 635 | | 627 | 635 |
| SAV2496 | | SasG | 1371 | 525 | 927 | | — | 1371 |
| SAV0023 | SA0022 | SasH | 772 | — | 772 | | 786 | 786 |
| SAV1731 | SA1552 | SasI | 895 | 891 | 891 | | 534 | 895 |
| SAV1129 | SA0976 | SasJ/IsdB | 645 | 645 | 645 | | 652 | 645 |
| | SA2381 | SasK | 198 | 211 | 211 | | — | 197 |
| | Np | SasL | — | 232 | — | | — | — |
| SAV1131 | SA0978 | IsdC | 227 | 227 | 227 | | 227 | 227 |

Proteins of the invention may be recombinant, or synthesized in vitro. Alternatively, a non-recombinant or recombinant protein may be isolated from bacteria. It is also contemplated that a bacteria containing such a variant may be implemented in compositions and methods of the invention. Consequently, a protein need not be isolated.

The term “functionally equivalent codon” is used herein to refer to codons that encode the same amino acid, such as the six codons for arginine or serine, and also refers to codons that encode biologically equivalent amino acids (see Table 2, below).

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, e.g., immunogenicity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

It is contemplated that in compositions of the invention, there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 ng or mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be Emp, Eap, and/or AdsA and may be used in combination with other polypeptide sequences described herein.

The present invention also discloses combinations of staphylococcal antigens which when combined, lead to the production of an immunogenic composition that is effective at treating or preventing staphylococcal infection. Staphylococcal infections progress through several different stages. For example, the staphylococcal life cycle involves commensal colonization, initiation of infection by accessing adjoining tissues or the bloodstream, anaerobic multiplication in the blood, interplay between S. aureus virulence determinants and the host defense mechanisms and induction of complications including endocarditis, metastatic abscess formation and sepsis syndrome. Different molecules on the surface of the bacterium will be involved in different steps of the infection cycle. Combinations of certain antigens can elicit an immune response which protects against multiple stages of staphylococcal infection. The effectiveness of the immune response can be measured either in animal model assays and/or using an opsonophagocytic assay.

A. Polypeptides and Polypeptide Production

The present invention describes polypeptides, peptides, and proteins and immunogenic fragments thereof for use in various embodiments of the present invention. For example, specific polypeptides are assayed for or used to elicit an immune response. In specific embodiments, all or part of the proteins of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide of the invention is inserted into an expression vector, transformed or transfected into an appropriate host cell and cultivated under conditions suitable for expression.

One embodiment of the invention includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

Another embodiment of the present invention uses autologous B lymphocyte cell lines, which are transfected with a viral vector that expresses an immunogen product, and more specifically, a protein having immunogenic activity. Other examples of mammalian host cell lines include, but are not limited to Vero and HeLa cells, other B- and T-cell lines, such as CEM, 721.221, H9, Jurkat, Raji, as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to HSV thymidine kinase, hypoxanthine-guanine phosphoribosyltransferase, and adenine phosphoribosyltransferase genes, in tk-, hgprt- or aprt-cells, respectively. Also, anti-metabolite resistance can be used as the basis of selection: for dhfr, which confers resistance to trimethoprim and methotrexate; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

Where a protein is specifically mentioned herein, it is preferably a reference to a native or recombinant protein or optionally a protein in which any signal sequence has been removed. The protein may be isolated directly from the staphylococcal strain or produced by recombinant DNA techniques. Immunogenic fragments of the protein may be incorporated into the immunogenic composition of the invention. These are fragments comprising at least 10 amino acids, 20 amino acids, 30 amino acids, 40 amino acids, 50 amino acids, or 100 amino acids, including all values and ranges there between, taken contiguously from the amino acid sequence of the protein. In addition, such immunogenic fragments are immunologically reactive with antibodies generated against the Staphylococcal proteins or with antibodies generated by infection of a mammalian host with Staphylococci.

Immunogenic fragments also includes fragments that when administered at an effective dose, (either alone or as a hapten bound to a carrier), elicit a protective immune response against Staphylococcal infection, in certain aspects it is protective against S. aureus and/or S. epidermidis infection. Such an immunogenic fragment may include, for example, the protein lacking an N-terminal leader sequence, and/or a transmembrane domain and/or a C-terminal anchor domain. In a preferred aspect the immunogenic fragment according to the invention comprises substantially all of the extracellular domain of a protein which has at least 85% identity, at least 90% identity, at least 95% identity, or at least 97-99% identity, including all values and ranges there between, to that a sequence selected over the length of the fragment sequence.

Also included in immunogenic compositions of the invention are fusion proteins composed of Staphylococcal proteins, or immunogenic fragments of staphylococcal proteins. Such fusion proteins may be made recombinantly and may comprise one portion of at least 2, 3, 4, 5 or 6 staphylococcal proteins. Alternatively, a fusion protein may comprise multiple portions of at least 1, 2, 3, 4 or 5 staphylococcal proteins. These may combine different Staphylococcal proteins and/or multiples of the same protein or protein fragment, or immunogenic fragments thereof in the same protein. Alternatively, the invention also includes individual fusion proteins of Staphylococcal proteins or immunogenic fragments thereof, as a fusion protein with heterologous sequences such as a provider of T-cell epitopes or purification tags, for example: (3-galactosidase, glutathione-S-transferase, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, and CRM197.

### II. Therapeutic Methods

Active immunization with vaccines and passive immunization with immunoglobulins are promising alternatives to classical small molecule (e.g., antibiotic) therapy. A few bacterial diseases that once caused widespread illness, disability and death can now be prevented through the use of vaccines. The vaccines are based on weakened (attenuated) or dead bacteria, components of the bacterial surface or inactivated toxins. The immune response raised by a vaccine is mainly directed to immunogenic structures; a limited number of proteins or sugar structures on the bacteria that are actively processed by the immune system.

A method of the present invention includes treatment for a disease or condition caused by or related to a bacterial pathogen, e.g., staphylococcus or bacillus. An immunogenic polypeptide, and/or antibody that binds the same, can be given to induce or provide a therapeutic response in a person infected with a bacteria or suspected of having been exposed to a bacteria. Methods may be employed with respect to individuals who have tested positive for exposure to staphylococcus or bacillus or who are deemed to be at risk for infection based on possible exposure.

The invention encompasses methods of treatment of staphylococcal infection, particularly hospital acquired nosocomial infections. In particular, the invention encompasses methods of treatment for bacterial infection, particularly bacteremia. The therapeutic compositions and vaccines of the invention are particularly advantageous in cases of elective surgery. Such patients will know the date of surgery in advance and could be inoculated or treated in advance. The immunogenic compositions and vaccines of the invention are also advantageous in inoculating health care workers, first responders, and the like.

In some embodiments, the treatment is administered in the presence of adjuvants or carriers or other staphylococcal antigens. Furthermore, in some examples, treatment comprises administration of other agents commonly used against bacterial infection, such as one or more antibiotics.

A. Vaccines

The present invention includes methods for preventing or ameliorating staphylococcal infections, particularly hospital acquired nosocomial infections. As such, the invention contemplates vaccines for use in both active and passive immunization embodiments. Immunogenic compositions, proposed to be suitable for use as a vaccine, may be prepared from immunogenic Emp, Eap and/or AdsA polypeptide(s), such as the full-length Emp, Eap and/or AdsA antigen or immunogenic fragments thereof. In other embodiments Emp, Eap and/or AdsA can be used in combination with other secreted virulence proteins, surface proteins or immunogenic fragments thereof, including EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or other staphylococcal antigen, peptide, or protein known to one of skill in the art. Preferably the antigenic material is extensively dialyzed to remove undesired small molecular weight molecules and/or lyophilized for more ready formulation into a desired vehicle.

Other viable and important options for a protein/peptide-based vaccine involve introducing nucleic acids encoding the antigen(s) as DNA vaccines. In this regard, recent reports described construction of recombinant vaccinia viruses expressing 10 contiguous minimal CTL epitopes (Thomson, 1996) or a combination of B cell, cytotoxic T-lymphocyte (CTL), and T-helper (Th) epitopes from several microbes (An, 1997), and successful use of such constructs to immunize mice for priming protective immune responses. Thus, there is ample evidence in the literature for successful utilization of peptides, peptide-pulsed antigen presenting cells (APCs), and peptide-encoding constructs for efficient in vivo priming of protective immune responses. The use of nucleic acid sequences as vaccines is exemplified in U.S. Pat. Nos. 5,958,895 and 5,620,896, each of which is incorporated herein by reference in its entirety.

The preparation of vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be conventionally administered by inhalation or parenterally by injection, e.g., subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The polypeptide, peptides and peptide-encoding DNA constructs may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may vary widely. Any of the conventional methods for administration of a vaccine are applicable. These are believed to include oral application on a solid physiologically acceptable base or in a physiologically acceptable dispersion, parenterally, by injection and the like. The dosage of the vaccine will depend on the route of administration and will vary according to the size and health of the subject.

In many instances, it will be desirable to have multiple administrations of the vaccine, usually at most, at least, or not exceeding six vaccinations, more usually four vaccinations, and typically one or more, usually at least about three vaccinations. The vaccinations will normally be at 1, 2, 3, 4, 5, 6, to 5, 6, 7, 8, 9, 10, 11, to 12 week intervals, including all values and ranges there between, more usually from three to five week intervals. Typically, periodic boosters at intervals of 1-5 years, usually three years, will be desirable to maintain protective levels of the antibodies. The course of the immunization may be followed by assays for antibodies against the antigens, as described supra, U.S. Pat. Nos. 3,791,932; 4,174,384 and 3,949,064, are illustrative of these types of assays.

The use of peptides for vaccination typically requires conjugation of the peptide to an immunogenic carrier protein, such as hepatitis B surface antigen, keyhole limpet hemocyanin, or bovine serum albumin, or an adjuvant. Methods for performing this conjugation are well known in the art.

1. Carriers

A given composition may vary in its immunogenicity. It is often necessary therefore to boost the host immune system, as may be achieved by coupling a peptide or polypeptide to a carrier. Carriers include, but are not limited to keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

2. Adjuvants

The immunogenicity of polypeptide or peptide compositions can be enhanced by the use of non-specific stimulators of the immune response, known as adjuvants. Suitable adjuvants include all acceptable immunostimulatory compounds, such as cytokines, toxins, or synthetic compositions.

A number of adjuvants can be used to enhance an antibody response against an Emp, Eap and/or AdsA peptide or any other antigen described herein. In other embodiments Emp, Eap and/or AdsA can be used in combination with EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, and/or SpA peptide or protein. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include IL-1, IL-2, IL-4, IL-7, IL-12, γ-interferon, GMCSP, BCG, aluminum salts, such as aluminum hydroxide or other aluminum compound, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM), and cell wall skeleton (CWS) in a 2% squalene/Tween 80 emulsion may also be used. MHC antigens may even be used. Others adjuvants or methods are exemplified in U.S. Pat. Nos. 6,814,971, 5,084,269, 6,656,462, each of which is incorporated herein by reference.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

A typical adjuvant is complete Freund's adjuvant (a non-specific stimulator of the immune response containing killed Mycobacterium tuberculosis), incomplete Freund's adjuvants, and aluminum hydroxide.

In some aspects, it is preferred that the adjuvant be selected to be a preferential inducer of either a Th1 or a Th2-type of response. High levels of Th1-type cytokines tend to favor the induction of cell mediated immune responses to a given antigen, while high levels of Th2-type cytokines tend to favor the induction of humoral immune responses to the antigen.

The distinction between Th1 and Th2-type immune response is not absolute. In reality an individual will support an immune response which is described as being predominantly Th1 or predominantly Th2. However, it is often convenient to consider the families of cytokines in terms of that described in murine CD4+T cell clones by Mosmann and Coffman (Mosmann, and Coffman, 1989). Traditionally, Th1-type responses are associated with the production of the INF-γ and IL-2 cytokines by T-lymphocytes. Other cytokines often directly associated with the induction of Th1-type immune responses are not produced by T-cells, such as IL-12.

In contrast, Th2-type responses are associated with the secretion of IL-4, IL-5, IL-6, IL-10.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, N.J.) and cytokines such as γ-interferon, IL-2, or IL-12 or genes encoding proteins involved in immune helper functions, such as B-7.

B. Antibodies And Passive Immunization

Direct administration of therapeutic immunoglobulins, also referred to as passive immunization, does not require an immune response from the patient and, therefore, gives immediate protection. In addition, passive immunization can be directed to bacterial structures that are not immunogenic and that are less specific to the organism. Passive immunization against pathogenic organisms has been based on immunoglobulins derived from sera of human or non-human donors.

One aspect of the invention is a method of preparing an immunoglobulin for use in prevention or treatment of staphylococcal infection comprising the steps of immunizing a recipient with the vaccine of the invention and isolating immunoglobulin or antibodies from the recipient. An immunoglobulin prepared by this method is a further aspect of the invention. A pharmaceutical composition comprising the immunoglobulin of the invention and a pharmaceutically acceptable carrier is a further aspect of the invention which could be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies. The antibodies can be isolated to the extent desired by well known techniques such as affinity chromatography (Harlow and Lane, 1988).

Antibodies can include antiserum preparations from a variety of commonly used animals, e.g., goats, primates, donkeys, swine, horses, guinea pigs, rats, or man. The animals are bled and serum recovered.

An immunoglobulin produced in accordance with the present invention can include whole antibodies, antibody fragments or subfragments. Antibodies can be whole immunoglobulins, chimeric antibodies or hybrid antibodies with dual specificity to two or more antigens of the invention. They may also be fragments, e.g., F(ab′)2, Fab′, Fab, Fv and the like including hybrid fragments. An immunoglobulin also includes natural, synthetic or genetically engineered proteins that act like an antibody by binding to specific antigens to form a complex. The term “immunoglobulin,” as used herein, includes all immunoglobulin classes and subclasses known in the art including IgA, IgD, IgE, IgG, and IgM, and their subclasses (isotypes), e.g., IgA1, IgA2, IgG1, IgG2, IgG3 and IgG4. Preferably, the immunoglobulins of the invention are human immunoglobulins. Also, an antigen-binding and/or variable domain comprising fragment of an immunoglobulin is meant. Antigen-binding fragments include, inter alia, Fab, F(ab′), F(ab′)2, Fv, dAb, Fd, complementarity-determining region (CDR) fragments, single-chain antibodies (scFv), bivalent single-chain antibodies, single-chain phage antibodies, diabodies, triabodies, tetrabodies, (poly)peptides that contain at least a fragment of an immunoglobulin that is sufficient to confer specific antigen binding to the (poly)peptide, etc.

An antigen composition or vaccine of the present invention can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins of the invention are particularly useful for treatment or prevention of staphylococcal disease in infants, immune compromised individuals or where treatment is required and there is no time for the individual to produce antibodies in response to vaccination.

An additional aspect of the invention is a pharmaceutical composition comprising one or more monoclonal antibodies (or fragments thereof; preferably human or humanized) reactive against constituents of the immunogenic composition of the invention, which could be used to treat or prevent infection by Gram positive bacteria, preferably staphylococci, more preferably S. aureus or S. epidermidis. Such pharmaceutical compositions comprise monoclonal antibodies that can be whole immunoglobulins of any class e.g. IgG, IgM, IgA, IgD or IgE, chimeric antibodies or hybrid antibodies with specificity to antigens of the invention. They may also be fragments, e.g., F(ab′)2, Fab′, Fab, Fv and the like including hybrid fragments.

Methods of making monoclonal antibodies are well known in the art and can include the fusion of splenocytes with myeloma cells (Kohler and Milstein, 1975; Harlow and Lane, 1988). Alternatively, monoclonal Fv fragments can be obtained by screening a suitable phage display library (Vaughan et al., 1998). Monoclonal antibodies may be human, humanized, or partly humanized by known methods.

C. Combination Therapy

The compositions and related methods of the present invention, particularly administration of a staphylococcal antigen, including a polypeptide or peptide of Emp, AdsA and/or Eap in combination with one or more of EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein peptide or protein to a patient/subject, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics. In one aspect, it is contemplated that a polypeptide vaccine and/or therapy is used in conjunction with a small molecule or non-peptide inhibitor of AdsA activity.

In one aspect, it is contemplated that a polypeptide vaccine and/or therapy is used in conjunction with antibacterial treatment. Alternatively, the therapy may precede or follow the treatment with the other agent by intervals ranging from minutes to weeks. In embodiments where the other agents and/or a proteins or polynucleotides are administered separately, one would generally ensure that a significant period of time did not expire between the time of each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and an immunogenic molecule or antibody given as part of an immune therapy regime, such as an antigen or an AdsA modulator, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the immunogenic compositions of the present invention to a patient/subject will follow general protocols for the administration of such compounds, taking into account the toxicity, if any, of the Emp, Eap, and/or AdsA composition, or composition of any other antigen or antigen combination described herein. It is expected that the treatment cycles would be repeated as necessary. It also is contemplated that various standard therapies, such as hydration, may be applied in combination with the described therapy.

### III. Pharmaceutical Compositions

In some embodiments, pharmaceutical compositions are administered to a subject. Different aspects of the present invention involve administering an effective amount of a composition to a subject. In some embodiments of the present invention, Emp, \ Eap and/or AdsA antigens in combination with members of the Ess pathway and including polypeptides or peptides of the Esa or Esx class, and/or members of sortase substrates, and/or secreted virulence factor and or polysaccharides may be administered to the patient to protect against or treat infection by one or more staphylococcus pathogens. Alternatively, an expression vector encoding one or more such polypeptides or peptides may be given to a patient as a preventative treatment. Additionally, such compounds can be administered in combination with an antibiotic. Such compositions will generally be dissolved or dispersed in a pharmaceutically acceptable carrier or aqueous medium.

As used herein, the term “pharmaceutically acceptable” or “pharmacologically acceptable” refer to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent. Pharmaceutically acceptable carrier includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-bacterial agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds of the present invention can be formulated for parenteral administration, e.g., formulated for injection via the intravenous, intramuscular, sub-cutaneous, or even intraperitoneal routes.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous administration. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

A. In Vitro, Ex Vivo, or in Vivo Administration

As used herein, the term in vitro administration refers to manipulations performed on cells removed from or outside of an animal, including, but not limited to cells in culture. The term ex vivo administration refers to cells which have been manipulated in vitro, and are subsequently administered to a living animal. The term in vivo administration includes all manipulations performed within an animal.

In certain aspects of the present invention, the compositions may be administered either in vitro, ex vivo, or in vivo. In certain in vitro embodiments, autologous B-lymphocyte cell lines are incubated with a virus vector of the instant invention for 24 to 48 hours or with Emp, Eap and/or AdsA, and/or EsaB, EsaC, EsxA, EsxB, SdrC, SdrD, SdrE, Hla or a variant thereof, IsdA, IsdB, ClfA, ClfB, IsdC, SasB, SasF, SpA, vWa, Coa, Ebh, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U55,801,234), SsaA, SSP-1, SSP-2, and/or Vitronectin binding protein (or any combination thereof). The transduced cells can then be used for in vitro analysis, or alternatively for ex vivo administration.

U.S. Pat. Nos. 4,690,915 and 5,199,942, both incorporated herein by reference, disclose methods for ex vivo manipulation of blood mononuclear cells and bone marrow cells for use in therapeutic applications.

B. Lipid Components and Moieties

In certain embodiments, the present invention concerns compositions comprising one or more lipids associated with a nucleic acid or a polypeptide/peptide. A lipid is a substance that is insoluble in water and extractable with an organic solvent. Compounds other than those specifically described herein are understood by one of skill in the art as lipids, and are encompassed by the compositions and methods of the present invention. A lipid component and a non-lipid may be attached to one another, either covalently or non-covalently.

A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

A nucleic acid molecule or a polypeptide/peptide associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid-associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL) or Superfect (Qiagen) complex is also contemplated.

In certain embodiments, a composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or any range therebetween, of a particular lipid, lipid type, or non-lipid component such as an adjuvant, antigen, peptide, polypeptide, sugar, nucleic acid or other material disclosed herein or as would be known to one of skill in the art. In a non-limiting example, a composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a non-lipid component. Thus, it is contemplated that compositions of the present invention may comprise any of the lipids, lipid types or other components in any combination or percentage range.

### IV. Polysaccharides

The immunogenic compositions of the invention may further comprise capsular polysaccharides including one or more of PIA (also known as PNAG) and/or S. aureus Type V and/or type VIII capsular polysaccharide and/or S. epidermidis Type I, and/or Type II and/or Type III capsular polysaccharide.

A. PIA (PNAG)

It is now clear that the various forms of staphylococcal surface polysaccharides identified as PS/A, PIA, and SAA are the same chemical entity—PNAG (Maira-Litran et al., 2004). Therefore the term PIA or PNAG encompasses all these polysaccharides or oligosaccharides derived from them.

PIA is a polysaccharide intercellular adhesin and is composed of a polymer of β-(1→6)-linked glucosamine substituted with N-acetyl and O-succinyl constituents. This polysaccharide is present in both S. aureus and S. epidermidis and can be isolated from either source (Joyce et al., 2003; Maira-Litran et al., 2002). For example, PNAG may be isolated from S. aureus strain MN8m (WO04/43407). PIA isolated from S. epidermidis is a integral constituent of biofilm. It is responsible for mediating cell-cell adhesion and probably also functions to shield the growing colony from the host's immune response.

The polysaccharide previously known as poly-N-succinyl-β-(1→6)-glucosamine (PNSG) was recently shown not to have the expected structure since the identification of N-succinylation was incorrect (Maira-Litran et al., 2002). Therefore the polysaccharide formally known as PNSG and now found to be PNAG is also encompassed by the term PIA. PIA (or PNAG) may be of different sizes varying from over 400 kDa to between 75 and 400 kDa to between 10 and 75 kDa to oligosaccharides composed of up to 30 repeat units (of β-(1→6)-linked glucosamine substituted with N-acetyl and O-succinyl constituents). Any size of PIA polysaccharide or oligosaccharide may be use in an immunogenic composition of the invention, however a size of over 40 kDa is preferred. Sizing may be achieved by any method known in the art, for instance by microfluidization, ultrasonic irradiation or by chemical cleavage (WO 03/53462, EP497524, EP497525). Preferred size ranges of PIA (PNAG) are 40-400 kDa, 40-300 kDa, 50-350 kDa, 60-300 kDa, 50-250 kDa and 60-200 kDa. PIA (PNAG) can have different degrees of acetylation due to substitution on the amino groups by acetate. PIA produced in vitro is almost fully substituted on amino groups (95-100%). Alternatively, a deacetylated PIA (PNAG) can be used having less than 60%, preferably less than 50%, 40%, 30%, 20%, 10% acetylation. Use of a deacetylated PIA (PNAG) is preferred since non-acetylated epitopes of PNAG are efficient at mediating opsonic killing of Gram positive bacteria, preferably S. aureus and/or S. epidermidis. Most preferably, the PIA (PNAG) has a size between 40 kDa and 300 kDa and is deacetylated so that less than 60%, 50%, 40%, 30% or 20% of amino groups are acetylated. The term deacetylated PNAG (dPNAG) refers to a PNAG polysaccharide or oligosaccharide in which less than 60%, 50%, 40%, 30%, 20% or 10% of the amino groups are acetylated.

In an embodiment, PNAG is a deaceylated to form dPNAG by chemically treating the native polysaccharide. For example, the native PNAG is treated with a basic solution such that the pH rises to above 10. For instance the PNAG is treated with 0.1-5 M, 0.2-4 M, 0.3-3 M, 0.5-2 M, 0.75-1.5 M or 1 M NaOH, KOH or NH₄OH. Treatment is for at least 10 to 30 minutes, or 1, 2, 3, 4, 5, 10, 15 or 20 hours at a temperature of 20-100, 25-80, 30-60 or 30-50 or 35-45° C. dPNAG may be prepared as described in WO 04/43405.

The polysaccharide(s) included in the immunogenic composition of the invention are preferably conjugated to a carrier protein as described below or alternatively unconjugated.

B. Type 5 and Type 8 Polysaccharides from Staphylococcus

Most strains of S. aureus that cause infection in man contain either Type 5 or Type 8 polysaccharides. Approximately 60% of human strains are Type 8 and approximately 30% are Type 5. The structures of Type 5 and Type 8 capsular polysaccharide antigens are described in Moreau et al., 1990 and Fournier et al., 1984). Both have FucNAcp in their repeat unit as well as ManNAcA which can be used to introduce a sulfhydryl group. The structures were reported as:

Type 5→4)-β-D-ManNAcA(3OAc)-(1→4)-α-L-FucNAc(1→3)-β-D-FucNAc-(1→

Type 8→3)-β-D-ManNAcA(4OAc)-(1→3)-α-L-FucNAc(1→3)-β-D-FucNAc-(1→

Recently (Jones, 2005) NMR spectroscopy revised the structures to:

Type 5→4)-β-D-ManNAcA-(1→4)-α-L-FucNAc(3OAc)-(1→3)-β-D-FucNAc-(1→

Type 8→3)-β-D-ManNAcA(4OAc)-(1→3)-α-L-FucNAc(1→3)-α-D-FucNAc(1→

Polysaccharides may be extracted from the appropriate strain of S. aureus using known methods, U.S. Pat. No. 6,294,177. ATCC 12902 is a Type 5 S. aureus strain and ATCC 12605 is a Type 8 S. aureus strain.

Polysaccharides are of native size or alternatively may be sized, for instance by microfluidisation, ultrasonic irradiation or by chemical treatment. The type 5 and 8 polysaccharides included in the immunogenic composition of the invention are preferably conjugated to a carrier protein as described below or are alternatively unconjugated. The immunogenic compositions of the invention alternatively contains either type 5 or type 8 polysaccharide.

C. S. Aureus 336 Antigen

In an embodiment, the immunogenic composition of the invention comprises the S. aureus 336 antigen described in U.S. Pat. No. 6,294,177. The 336 antigen comprises β-linked hexosamine, contains no O-acetyl groups and specifically binds to antibodies to S. aureus Type 336 deposited under ATCC 55804. In an embodiment, the 336 antigen is a polysaccharide which is of native size or alternatively may be sized, for instance by microfluidisation, ultrasonic irradiation or by chemical treatment. The invention also covers oligosaccharides derived from the 336 antigen. The 336 antigen, where included in the immunogenic composition of the invention is preferably conjugated to a carrier protein as described below or are alternatively unconjugated.

D. Type I, II and III Polysaccharides from S. Epidermidis

Strains ATCC-31432, SE-360 and SE-10 of S. epidermidis are characteristic of three different capsular types, I, II and III respectively (Ichiman and Yoshida, 1981). Capsular polysaccharides extracted from each serotype of S. epidermidis constitute Type I, II, and III polysaccharides. Polysaccharides may be extracted by several methods including the method described in U.S. Pat. No. 4,197,290 or as described in Ichiman et al., 1991.

In one embodiment of the invention, the immunogenic composition comprises type I and/or II and/or III polysaccharides or oligosaccharides from S. epidermidis.

Polysaccharides are of native size or alternatively may be sized, for instance by microfluidisation, ultrasonic irradiation or chemical cleavage. The invention also covers oligosaccharides extracted from S. epidermidis strains. These polysaccharides are unconjugated or are preferably conjugated as described below.

E. Conjugation of Polysaccharides

Amongst the problems associated with the use of polysaccharides in vaccination, is the fact that polysaccharides per se are poor immunogens. It is preferred that the polysaccharides utilized in the invention are linked to such a protein carrier to improve immunogenicity. Examples of such carriers which may be conjugated to polysaccharide immunogens include the Diphtheria and Tetanus toxoids (DT, DT CRM197 and TT respectively), Keyhole Limpet Haemocyanin (KLH), and the purified protein derivative of Tuberculin (PPD), Pseudomonas aeruginosa exoprotein A (rEPA), protein D from Haemophilus influenzae, pneumolysin or fragments of any of the above. Fragments suitable for use include fragments encompassing T-helper epitopes. In particular the protein D fragment from H. influenza will preferably contain the N-terminal ⅓ of the protein. Protein D is an IgD-binding protein from Haemophilus influenzae (EP 0 594 610 B1) and is a potential immunogen. In addition, staphylococcal proteins/antigens may be used as carrier protein in the polysaccharide conjugates of the invention.

A carrier protein that would be particularly advantageous to use in the context of a staphylococcal vaccine is staphylococcal alpha toxoid. The native form may be conjugated to a polysaccharide since the process of conjugation reduces toxicity. Preferably a genetically detoxified alpha toxins such as the His35Leu or His35Arg variants are used as carriers since residual toxicity is lower. Alternatively the alpha toxin is chemically detoxified by treatment with a cross-linking reagent, formaldehyde or glutaraldehyde.

The polysaccharides may be linked to the carrier protein(s) by any known method (for example, U.S. Pat. Nos. 4,372,945, 4,474,757, and 4,356,170).

Preferably, CDAP conjugation chemistry is carried out (see WO95/08348). In CDAP, the cyanylating reagent 1-cyano-dimethylaminopyridinium tetrafluoroborate (CDAP) is preferably used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide is solubilized in water or a saline solution. CDAP is dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

Conjugation preferably involves producing a direct linkage between the carrier protein and polysaccharide. Optionally a spacer (such as adipic dihydride (ADH)) may be introduced between the carrier protein and the polysaccharide.

### V. Immune Response and Assays

As discussed above, the invention concerns evoking or inducing an immune response in a subject against an Emp, Eap and/or AdsA polypeptide. In other embodiments an immune response to other peptides or antigens can be evoked or induced, including EsaB, EsaC, EsxA, EsxB, SdrC, SdrD, SdrE, Hla or a variant thereof, IsdA, IsdB, ClfA, ClfB, IsdC, SasB, SasF, SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or any other Staphylococcal peptide or protein. In one embodiment, the immune response can protect against or treat a subject (e.g., limiting abscess persistence) having, suspected of having, or at risk of developing an infection or related disease, particularly those related to staphylococci. One use of the immunogenic compositions of the invention is to prevent nosocomial infections by inoculating or treating a subject prior to hospital treatment.

A. Immunoassays

The present invention includes the implementation of serological assays to evaluate if an immune response is induced or evoked by Emp, Eap and/or AdsA and any other polypeptide or peptide agent described herein. There are many types of immunoassays that can be implemented. Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful. In one example, the antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove nonspecifically bound immune complexes, the bound antigen or antibody may be detected. Detection is generally achieved by the addition of another antibody, specific for the desired antigen or antibody, that is linked to a detectable label. This type of ELISA is known as a “sandwich ELISA”. Detection also may be achieved by the addition of a second antibody specific for the desired antigen, followed by the addition of a third antibody that has binding affinity for the second antibody, with the third antibody being linked to a detectable label.

Variations on ELISA techniques are known to those of skill in the art. In one such variation, the samples suspected of containing a target antigen or antibody are immobilized onto the well surface and then contacted with the antibodies or antigens of the invention. After binding and appropriate washing, the bound immune complexes are detected. Where the initial antigen specific antibodies are linked to a detectable label, the immune complexes may be detected directly. Again, the immune complexes may be detected using a second antibody that has binding affinity for the first antigen specific antibody, with the second antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non specifically bound species, and detecting the bound immune complexes.

In ELISAs, it is more customary to use a secondary or tertiary detection means rather than a direct procedure. Thus, after binding of the antigen or antibody to the well, coating with a non reactive material to reduce background, and washing to remove unbound material, the immobilizing surface is contacted with the clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

After all incubation steps in an ELISA are followed, the contacted surface is washed so as to remove non complexed material. Washing often includes washing with a solution of PBS/Tween, or borate buffer. Following the formation of specific immune complexes between the test sample and the originally bound material, and subsequent washing, the occurrence of even minute amounts of immune complexes may be determined.

To provide a detecting means, the second or third antibody can have an associated label to allow detection. In certain aspects, this label will be an enzyme that will generate color development upon incubating with an appropriate chromogenic substrate. Thus, for example, one will desire to contact and incubate the first or second immune complex with a urease, glucose oxidase, alkaline phosphatase, or hydrogen peroxidase conjugated antibody for a period of time and under conditions that favor the development of further immune complex formation, e.g., incubation for 2 hours at room temperature in a PBS containing solution such as PBS Tween.

After incubation with the labeled antibody, and subsequent to washing to remove unbound material, the amount of label is quantified, e.g., by incubation with a chromogenic substrate such as urea and bromocresol purple or 2,2′ azino-di(3-ethyl benzthiazoline-6-sulfonic acid [ABTS] and H₂O₂, in the case of peroxidase as the enzyme label. Quantification is then achieved by measuring the degree of color generation, e.g., using a visible spectra spectrophotometer. Alternatively, the label may be a chemiluminescent label (see, U.S. Pat. Nos. 5,310,687, 5,238,808 and 5,221,605).

B. Diagnosis of Bacterial Infection

In addition to the use of proteins, polypeptides, and/or peptides, as well as antibodies binding these polypeptides, proteins, and/or peptides to treat or prevent infection as described above, the present invention contemplates the use of these polypeptides, proteins, peptides, and/or antibodies in a variety of ways, including the detection of the presence of Staphylococci and diagnosing an infection, whether in a patient or on medical equipment which may also become infected. In accordance with the invention, a preferred method of detecting the presence of infections involves the steps of obtaining a sample suspected of being infected by one or more staphylococcal bacteria species or strains, such as a sample taken from an individual, for example, from one's blood, saliva, tissues, bone, muscle, cartilage, or skin. Following isolation of the sample, diagnostic assays utilizing the polypeptides, proteins, peptides, and/or antibodies of the present invention may be carried out to detect the presence of staphylococci, and such assay techniques for determining such presence in a sample are well known to those skilled in the art and include methods such as radioimmunoassay, western blot analysis and ELISA assays. In general, in accordance with the invention, a method of diagnosing an infection is contemplated wherein a sample suspected of being infected with staphylococci has added to it the polypeptide, protein, peptide, antibody, or monoclonal antibody in accordance with the present invention, and staphylococci are indicated by antibody binding to the polypeptides, proteins, and/or peptides, or polypeptides, proteins, and/or peptides binding to the antibodies in the sample.

Accordingly, antibodies in accordance with the invention may be used for the prevention of infection from staphylococcal bacteria, for the treatment of an ongoing infection, or for use as research tools. The term “antibodies” as used herein includes monoclonal, polyclonal, chimeric, single chain, bispecific, human, simianized, and humanized or primatized antibodies as well as Fab fragments, such as those fragments which maintain the binding specificity of the antibodies, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. Specific examples of the generation of an antibody to a bacterial protein can be found in U.S. Patent Publication 20030153022, which is incorporated herein by reference in its entirety.

Any of the above described polypeptides, proteins, peptides, and/or antibodies may be labeled directly with a detectable label for identification and quantification of staphylococcal bacteria. Labels for use in immunoassays are generally known to those skilled in the art and include enzymes, radioisotopes, and fluorescent, luminescent and chromogenic substances, including colored particles such as colloidal gold or latex beads. Suitable immunoassays include ELISAs.

C. Protective Immunity

In some embodiments of the invention, proteinaceous compositions confer protective immunity on a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein in the specification and in the claims section that follows, the term polypeptide and peptide refers to a stretch of amino acids covalently linked there amongst via peptide bonds. Different polypeptides have different functionalities according to the present invention. While according to one aspect, a polypeptide is derived from an immunogen designed to induce an active immune response in a recipient, according to another aspect of the invention, a polypeptide is derived from an antibody which results following the elicitation of an active immune response, in, for example, an animal, and which can serve to induce a passive immune response in the recipient. In both cases, however, the polypeptide can be encoded by a polynucleotide according to any possible codon usage.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate or polypeptide of the invention in a recipient patient. Such a response can be an active response induced by administration of immunogen or a passive response induced by administration of antibody, antibody containing material, or primed T-cells.

A cellular immune response is elicited by the presentation of polypeptide antigens or epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity.

As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response.

A monoclonal or polyclonal antibody composition may be used in passive immunization for the prevention or treatment of infection by organisms that carry or may be exposed to the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition of the present invention can be administered to a subject who then acts as a source or donor for globulin, produced in response to challenge with the antigenic composition (“hyperimmune globulin”), that contains antibodies directed against Staphylococcus or other organism. A subject thus treated would donate plasma from which hyperimmune globulin would then be obtained, via conventional plasma-fractionation methodology, and administered to another subject in order to impart resistance against or to treat staphylococcus infection. Hyperimmune globulins according to the invention are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

In certain aspects, methods include treating or preventing infection by administering the antibody compositions, such as antibodies that bind the above-described antigens, to a subject in need thereof. A target patient population for the treatment and prevention of infection includes mammals, such as humans, who are infected with or at risk of being infected by bacterial pathogens. In one embodiment, the infection to be treated or prevented is an S. aureus infection, including an infection of methicillin-resistant S. aureus or S. aureus producing alpha-toxin, or an S. epidermidis infection.

In accordance with one embodiment, the invention provides a method for treating or preventing an S. aureus infection using compositions comprising one or more S. aureus AdsA, Emp, Eap, EsaB, EsaC, EsxA, EsxB, SdrC, SdrD, SdrE, Hla or a variant thereof, IsdA, IsdB, ClfA, ClfB, IsdC, SasB, SasF, SpA, Ebh, vWa, Coa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein antibodies and a pharmaceutically acceptable carrier. The S. aureus antibody can bind to any of those antigens described above. In one embodiment, the antibody composition is a antibody composition or a hyperimmune composition. In another embodiment, the antibodies are recombinant, human, or humanized antibodies. In yet another embodiment, the antibodies are monoclonal antibodies, or fragments thereof.

A therapeutically or prophylactically effective amount of the antibody compositions can be determined by methods that are routine in the art. Skilled artisans will recognize that the amount may vary according to the particular antibodies within the composition, the concentration of antibodies in the composition, the frequency of administration, the severity of infection to be treated or prevented, and subject details, such as age, weight and immune condition. In some embodiments, the dosage will be at least 1, 5, 10, 25, 50, or 100 μg or mg of antibody composition per kilogram of body weight (mg/kg), including at least 100 mg/kg, at least 150 mg/kg, at least 200 mg/kg, at least 250 mg/kg, at least 500 mg/kg, at least 750 mg/kg and at least 1000 mg/kg. Dosages for monoclonal antibody compositions typically may be lower, such as 1/10 of the dosage of an antibody composition, such as at least about 1, 5, 10, 25, or 50 μg or mg/kg, at least about 10 mg/kg, at least about 15 mg/kg, at least about 20 mg/kg, or at least about 25 mg/kg. The route of administration may be any of those appropriate for a passive vaccine. Thus, intravenous, subcutaneous, intramuscular, intraperitoneal, inhalation, and other routes of administration are envisioned. As noted above, a therapeutically or prophylactically effective amount of antibody is an amount sufficient to achieve a therapeutically or prophylactically beneficial effect.

A protective antibody composition may neutralize and/or prevent infection. A protective antibody composition may comprise amounts of AdsA, Emp, Eap, EsaB, EsaC, EsxA, EsxB, SdrC, SdrD, SdrE, Hla or a variant thereof, IsdA, IsdB, ClfA, ClfB, IsdC, SasB, SasF, SpA, Ebh, Coa, vWa, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein antibodies that are not protective on their own, but which, in combination, yield a protective antibody composition.

The antibody composition may be administered in conjunction with an anti-infective agent, an antibiotic agent, and/or an antimicrobial agent, in a combination therapy. Anti-infective agents include, but are not limited to vancomycin and lysostaphin. Antibiotic agents and antimicrobial agents include, but are not limited to penicillinase-resistant penicillins, cephalosporins and carbapenems, including vancomycin, lysostaphin, penicillin G, ampicillin, oxacillin, nafcillin, cloxacillin, dicloxacillin, cephalothin, cefazolin, cephalexin, cephradine, cefamandole, cefoxitin, imipenem, meropenem, gentamycin, teicoplanin, lincomycin and clindamycin. The dosages of these antibiotics are well known in the art. See for example, Merck Manual Of Diagnosis And Therapy, §13, Ch. 157, 100^{th} Ed. (Beers & Berkow, eds., 2004). The anti-infective, antibiotic and/or antimicrobial agents may be combined prior to administration, or administered concurrently or sequentially with active or passive immunotherapies described herein.

In some embodiments, relatively few doses of antibody composition are administered, such as one or two doses, and conventional antibiotic therapy is employed, which generally involves multiple doses over a period of days or weeks. Thus, the antibiotics can be taken one, two or three or more times daily for a period of time, such as for at least 5 days, 10 days or even 14 or more days, while the antibody composition is usually administered only once or twice. However, the different dosages, timing of dosages and relative amounts of antibody composition and antibiotics can be selected and adjusted by one of ordinary skill in the art.

For purposes of this specification and the accompanying claims the terms “epitope” and “antigenic determinant” are used interchangeably to refer to a site on an antigen to which B and/or T cells respond or recognize B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit, goat, sheep or human, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

In order to produce monoclonal antibodies, hyperimmunization of an appropriate donor, generally a mouse, with the antigen is undertaken. Isolation of splenic antibody producing cells is then carried out. These cells are fused to a cell characterized by immortality, such as a myeloma cell, to provide a fused cell hybrid (hybridoma) which can be maintained in culture and which secretes the required monoclonal antibody. The cells are then cultured, in bulk, and the monoclonal antibodies harvested from the culture media for use. By definition, monoclonal antibodies are specific to a single epitope. Monoclonal antibodies often have lower affinity constants than polyclonal antibodies raised against similar antigens for this reason.

Monoclonal antibodies may also be produced ex-vivo by use of primary cultures of splenic cells or cell lines derived from spleen (Anavi, 1998). In order to produce recombinant antibody (see generally Huston et al., 1991; Johnson et al., 1991; Mernaugh et al., 1995), messenger RNAs from antibody producing B-lymphocytes of animals, or hybridoma are reverse-transcribed to obtain complementary DNAs (cDNAs). Antibody cDNA, which can be full length or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. For a general discussion of conjugation of fluorescent or enzymatic moieties see Johnstone et al. (1982). The binding of antibodies to a solid support substrate is also well known in the art (Harlow et al., 1988; Borrebaeck, 1992).

As used herein and in the claims, the phrase “an immunological portion of an antibody” include a Fab fragment of an antibody, a Fv fragment of an antibody, a heavy chain of an antibody, a light chain of an antibody, an unassociated mixture of a heavy chain and a light chain of an antibody, a heterodimer consisting of a heavy chain and a light chain of an antibody, a catalytic domain of a heavy chain of an antibody, a catalytic domain of a light chain of an antibody, a variable fragment of a light chain of an antibody, a variable fragment of a heavy chain of an antibody, and a single chain variant of an antibody, which is also known as scFv. In addition, the term includes chimeric immunoglobulins, which are the expression products of fused genes derived from different species. One of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VI. Nucleic Acids

In certain embodiments, the present invention concerns recombinant polynucleotides encoding the proteins, polypeptides and peptides of the invention. The nucleic acid sequences for Emp, Eap or AdsA, and other bacterial proteins including, but not limited to EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, Coa, vWa, Ebh, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein are included.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, mRNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs of a polypeptide of the invention. It also is contemplated that a particular polypeptide may be encoded by nucleic acids containing variations having slightly different nucleic acid sequences but, nonetheless, encode the same or substantially similar protein (see Table 2 above).

In particular embodiments, the invention concerns isolated nucleic acid segments and recombinant vectors incorporating nucleic acid sequences that encode Emp, Eap and/or AdsA, that may also be in combination with EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein. Thus, an isolated nucleic acid segment or vector containing a nucleic acid segment may encode, for example, Emp Eap and/or AdsA, and/or EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein that is/are immunogenic. The term “recombinant” may be used in conjunction with a polypeptide or the name of a specific polypeptide, and this generally refers to a polypeptide produced from a nucleic acid molecule that has been manipulated in vitro or that is a replication product of such a molecule.

In other embodiments, the invention concerns isolated nucleic acid segments and recombinant vectors incorporating nucleic acid sequences that encode a peptide or polypeptide to generate an immune response in a subject. In various embodiments the nucleic acids of the invention may be used in genetic vaccines.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain other embodiments, the invention concerns isolated nucleic acid segments and recombinant vectors that include within their sequence a contiguous nucleic acid sequence from SEQ ID NO:1 (Emp), SEQ ID NO:3 (Eap), SEQ ID NO:5 (EsxA), SEQ ID NO:7 (EsxB), SEQ ID NO:9 (SdrD), SEQ ID NO:11 (SdrE), SEQ ID NO:13 (IsdA), SEQ ID NO:15 (IsdB), SEQ ID NO:17 (SpA), SEQ ID NO:19 (ClfB), SEQ ID NO:21 (IsdC), SEQ ID NO:23 (SasF), SEQ ID NO:25 (SdrC), SEQ ID NO:27 (ClfA), SEQ ID NO:29 (EsaB), SEQ ID NO:31 (EsaC), SEQ ID NO:33 (SasB), or SEQ ID NO:35 (Sas) or any other nucleic acid sequences encoding secreted virulence factors and/or surface proteins.

In certain embodiments, the present invention provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this invention using the methods described herein (e.g., BLAST analysis using standard parameters). In certain aspects, the isolated polynucleotide of the invention will comprise a nucleotide sequence encoding a polypeptide that has at least 90%, preferably 95% and above, identity to an amino acid sequence of the invention, over the entire length of the sequence; or a nucleotide sequence complementary to said isolated polynucleotide.

The invention also contemplates the use of polynucleotides which are complementary to all the above described polynucleotides.

The invention also provides for the use of a fragment of a polynucleotide of the invention which when administered to a subject has the same immunogenic properties as a polynucleotide.

The invention also provides for the use of a polynucleotide encoding an immunological fragment of a protein of the invention as hereinbefore defined.

A. Vectors

Polypeptides of the invention may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding an Emp, Eap or AdsA polypeptide the vector can encode an EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or any other Staphylococcal peptides or proteins, a vector may encode polypeptide sequences such as a tag or immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

Vectors of the invention may be used in a host cell to produce an Emp or Eap or AdsA polypeptide. In certain aspects the vectors may also produce EsaB, EsaC, EsxA, EsxB, SdrC, SdrD, SdrE, Hla or a variant thereof, IsdA, IsdB, ClfA, ClfB, IsdC, SasB, SasF, Ebh, Coa, vWa, SpA, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or any other Staphylococcal peptides or proteins that may subsequently be purified for administration to a subject or the vector may be purified for direct administration to a subject for expression of the protein in the subject.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference).

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ a and/or DQ β (Sullivan et al., 1987), β Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DRα (Sherman et al., 1989), 13-Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), α-Fetoprotein (Godbout et al., 1988; Campere et al., 1989), γ-Globin (Bodine et al., 1987; Perez-Stable et al., 1990), β-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), α1-Antitrypain (Latimer et al., 1990), H₂B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foecking et al., 1986), Gibbon Ape Leukemia Virus (Holbrook et al., 1987; Quinn et al., 1989).

Inducible elements include, but are not limited to MT II—Phorbol Ester (TFA)/Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)—Glucocorticoids (Huang et al., 1981; Lee et al., 1981; Majors et al., 1983; Chandler et al., 1983; Lee et al., 1984; Ponta et al., 1985; Sakai et al., 1988); β-Interferon—poly(rI)x/poly(rc) (Tavernier et al., 1983); Adenovirus 5 E2—ElA (Imperiale et al., 1984); Collagenase—Phorbol Ester (TPA) (Angel et al., 1987a); Stromelysin—Phorbol Ester (TPA) (Angel et al., 1987b); SV40—Phorbol Ester (TPA) (Angel et al., 1987b); Murine MX Gene—Interferon, Newcastle Disease Virus (Hug et al., 1988); GRP78 Gene—A23187 (Resendez et al., 1988); α-2-Macroglobulin—IL-6 (Kunz et al., 1989); Vimentin—Serum (Rittling et al., 1989); MHC Class I Gene H-2 Kb—Interferon (Blanar et al., 1989); HSP70—ElA/SV40 Large T Antigen (Taylor et al., 1989, 1990a, 1990b); Proliferin—Phorbol Ester/TPA (Mordacq et al., 1989); Tumor Necrosis Factor—PMA (Hensel et al., 1989); and Thyroid Stimulating Hormone a Gene—Thyroid Hormone (Chatterjee et al., 1989).

The particular promoter that is employed to control the expression of a peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human, or viral promoter.

In various embodiments, the human cytomegalovirus (CMV) immediate early gene promoter, the SV40 early promoter, or the Rous sarcoma virus long terminal repeat can be used to obtain high level expression of an Emp, AdsA and/or Eap polynucleotide. In other embodiments Emp, Eap and/or AdsA can be used expressed in combination with EsaB, EsaC, EsxA, EsxB, SdrC, SdrD, SdrE, Hla or a variant thereof, IsdA, IsdB, ClfA, ClfB, IsdC, SasB, SasF, Spa, vWa, Coa, Ebh, 52 kDa vitronectin binding protein (WO 01/60852), Aaa, Aap, Ant, autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/P isA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF(WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein or any other bacterial polypeptide. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of an Emp, Eap and/or AdsA polypeptide for eliciting an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters.

2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Amplification of Nucleic Acids

Nucleic acids used as a template for amplification may be isolated from cells, tissues or other samples according to standard methodologies (Sambrook et al., 2001). In certain embodiments, analysis is performed on samples without substantial purification of the template nucleic acid. The nucleic acid may be genomic DNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

E. Methods of Gene Transfer

Suitable methods for nucleic acid delivery to effect expression of compositions of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); or by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference). Through the application of techniques such as these, organelle(s), cell(s), tissue(s) or organism(s) may be stably or transiently transformed.

### VII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Envelope Proteins Associated with Abscess Formation and Vaccine Protection

### A. Results

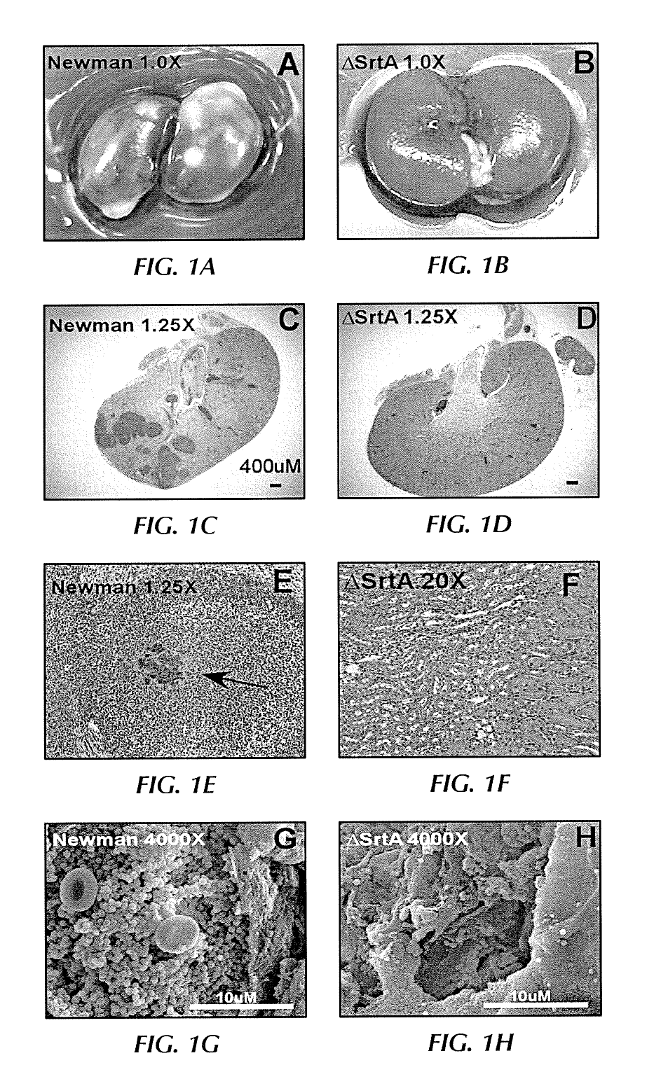

The inventors sought to study the pathogenesis of staphylococcal abscess formation and identify the bacterial factors that enable staphylococcal survival and proliferation within this lesion. The studies used the murine renal abscess model, wherein mice are infected with a sub-lethal dose of S. aureus to develop a sustained infection (Burts et al., 2005). Mice were killed on the fifth day post-infection, their kidneys excised and subjected to histopathology of thin-sectioned hemotoxylin-eosin stained tissue or to enumeration of staphylococcal load by plating tissue homogenate for colony forming units (CFU). In comparison to the wild-type clinical isolated S. aureus Newman (Baba et al., 2008), an isogenic variant with a transposon insertion in the structural gene for sortase A failed to form abscesses (FIGS. 1B, 1D, and 1F). Sortase A, which anchors a large spectrum of surface proteins with LPXTG motif sorting signals to the cell wall envelope, is responsible for the surface display of many different virulence factors (Mazmanian et al., 2000; Mazmanian et al., 1999). To quantify abscess formation, kidneys were visually inspected, and each individual organ was given a score of one (surface abscesses present) or zero (absent). The final sum was divided by the total number of kidneys to obtain a fractional value. In addition, three randomly chosen right-sided kidneys were placed in 10% formalin overnight, embedded, sectioned, and stained with hematoxylin and eosin. For each kidney, four sagittal sections at 200 μM intervals were viewed by microscopy. If a lesion was observed in any plane of inspection, the organ was judged positive for abscess formation. Mice infected with 3×10⁷ CFU/ml S. aureus Newman displayed visible lesions on 16/20 kidneys (80% surface abscess) and were positive for abscess formation in 3/3 kidneys examined for histopathology (Table 3). In contrast, mice infected with the AsrtA mutant presented with 0% surface abscesses and 0/3 histological lesions (Table 3).

TABLE 3
Recovered Recovered CFUs with standard error, log reduction
with respect to Newman, P-value (student's t-test), % surface
abscesses observed, # histological abscesses observed.

| | | | | Kidney abscess formation | |
|---|---|---|---|---|---|
| | Staphylococcal load in kidney tissue | | | | Histo- |
| Strain | log₁₀ CFU g⁻¹ | P-value | Reduction | Surface % | pathology |
| wild-type | 6.040 ± 0.095 | — | — | 80 | 3 |
| ΔsrtA | 3.911 ± 0.389 | 0.0002 | 1.830 | 0 | 0 |

Surface protein genes

| | | | | | |
|---|---|---|---|---|---|
| sdrD | 3.629 ± 0.758 | 0.0040 | 2.411 | 22 | 1 |
| isdB | 4.253 ± 0.510 | 0.0027 | 1.790 | 5 | 1 |
| clfB | 4.624 ± 0.446 | 0.0067 | 1.398 | 30 | 2 |
| isdA | 4.723 ± 0.280 | 0.0002 | 1.320 | 15 | 1 |
| sasB | 5.089 ± 0.448 | 0.0433 | 0.951 | 38 | 2 |
| sasD | 5.206 ± 0.375 | 0.0371 | 0.833 | 45 | 1 |
| sasC | 5.222 ± 0.400 | 0.0594 | 0.824 | 50 | 2 |
| sasF | 5.421 ± 0.360 | 0.1051 | 0.619 | 30 | 2 |
| sasA | 5.431 ± 0.403 | 0.1217 | 0.609 | 40 | 2 |
| sasG | 5.433 ± 0.360 | 0.1051 | 0.607 | 40 | 2 |
| isdC | 5.498 ± 0.292 | 0.0945 | 0.541 | 33 | 2 |
| fnbpB | 5.530 ± 0.359 | 0.1856 | 0.511 | 30 | 1 |
| sasI | 5.599 ± 0.416 | 0.2681 | 0.441 | 38 | 2 |
| spa | 5.681 ± 0.455 | 0.4487 | 0.359 | 10 | 1 |
| fnbpA | 5.751 ± 0.322 | 0.3800 | 0.289 | 40 | 2 |
| sdrE | 5.848 ± 0.334 | 0.5686 | 0.192 | 61 | 3 |
| clfA | 5.898 ± 0.296 | 0.1470 | (+) 0.472 | 40 | 2 |

PNAG (PIA) genes

| | | | | | |
|---|---|---|---|---|---|
| icaA | 5.326 ± 0.452 | 0.1122 | 0.822 | 40 | 3 |
| icaB | 5.894 ± 0.306 | 0.4917 | 0.254 | 35 | 2 |
| icaC | 5.651 ± 0.441 | 0.3004 | 0.497 | 35 | 2 |
| icaD | 5.886 ± 0.278 | 0.4394 | 0.262 | 45 | 2 |
| icaR | 6.201 ± 0.309 | 0.8837 | −0.053 | 60 | 2 |
| ica:tet | 5.692 ± 0.280 | 0.1909 | 0.456 | 55 | 2 |

Envelope protein genes

| | | | | | |
|---|---|---|---|---|---|
| eap | 6.530 ± 0.385 | 0.1217 | (+)0.49 | 50 | 2 |
| emp | 5.716 ± 0.080 | 0.1051 | 0.324 | 10 | 1 |
| eap/ΔsrtA | 4.708 ± 0.545 | 0.0129 | 1.332 | 0 | 0 |
| emp/ΔsrtA | 3.165 ± 0.496 | 5.53 × 10⁻⁴ | 2.875 | 0 | 0 |

Capsular polysaccharide genes

| | | | | | |
|---|---|---|---|---|---|
| capO | — | — | — | — | — |

^{a}Means of staphylococcal load in colony forming units (CFU) calculated as log₁₀ CFU g⁻¹ in homogenized renal tissues 5 days following infection in cohorts of fifteen BALB/c mice per challenge strain. Standard error of the means (±SEM) is indicated.
^{b}Statistical significance was calculated with the students t-test and P-values recorded.
^{c}Reduction in bacterial load calculated as log₁₀ CFU g⁻¹.
^{c}Abscess formation in kidney tissues five days following infection. was measured by macroscopic inspection (% positive) and histopathology of hematoxylene-eosine stained, thin sectioned tissues from three animals, whereby positive tissues were recorded as fractional values ( 3/n).

Recovered CFUs with standard error, log reduction with respect to Newman, P-value (student's t-test), % surface abscesses observed, # histological abscesses observed.

Scanning electron microscopy was used to examine infected tissues. Kidneys were sectioned, fixed, dehydrated in hexamethyldisilazane (HMDS), and sputter coated with 80% Pt/20% Pd prior to viewing. Kidney tissue infected with S. aureus Newman harbored bacteria within a central region of the abscess. Wild-type staphylococci were found in tightly associated lawns (FIG. 1G), contained by a fibrous structure that is internal to the larger fibrin capsule. These staphylococcal nests are devoid of leukocytes and appear to be embedded by an adhesive extracellular matrix. Kidneys tissue infected with the srtA mutant also harbored staphylococci, however the bacteria were dispersed throughout healthy renal tissue and significantly reduced in number compared to the wild-type (FIG. 1H).

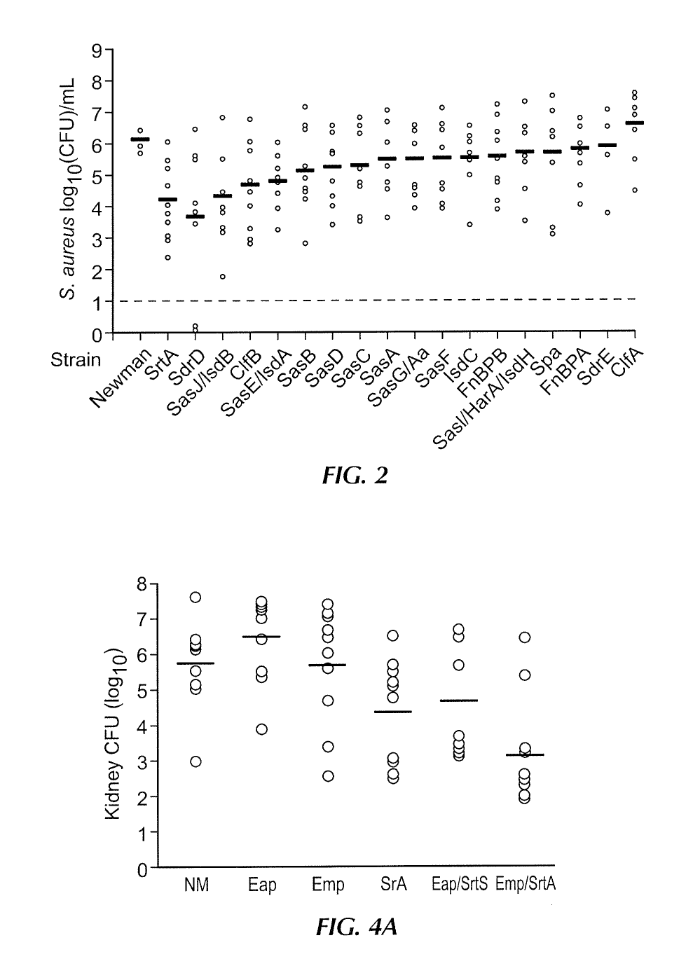

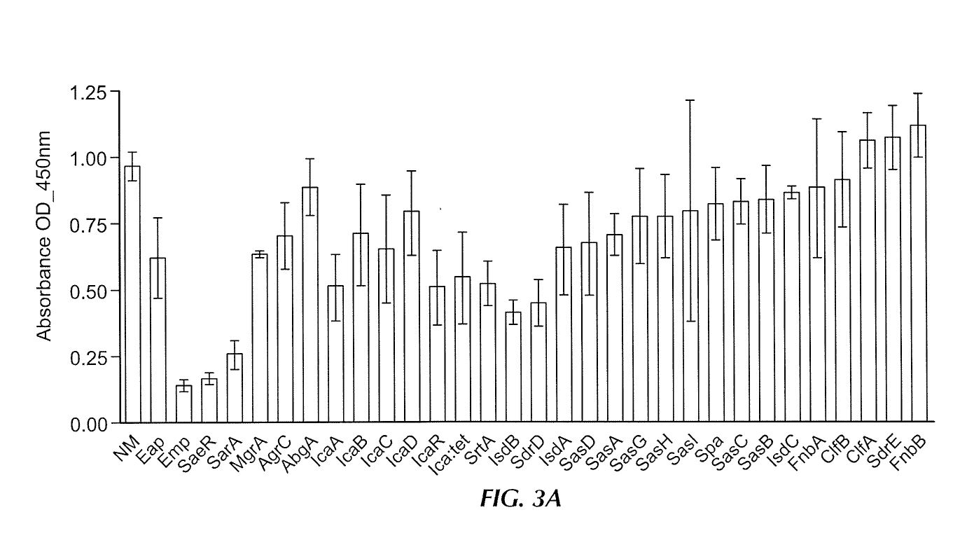

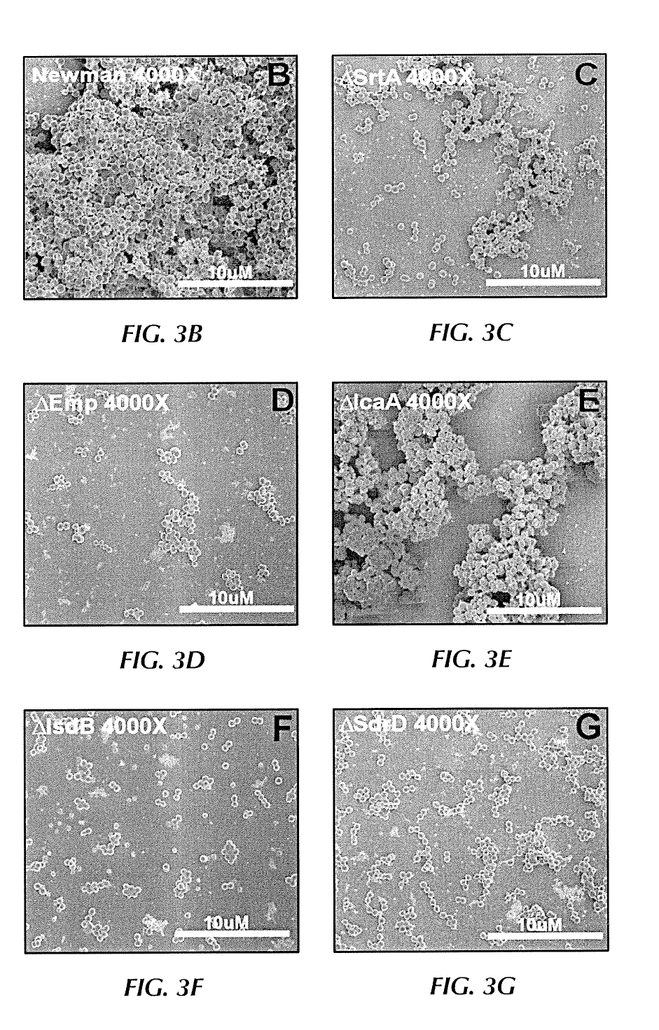

To identify the specific sortase A substrates responsible for abscess formation, the inventors transduced bursa aurealis insertions in 17 sortase substrate genes (Bae et al., 2004) into S. aureus Newman and screened the variants for virulence defects. Mutations in sdrD, isdB, clfB, isdA, sasB, and sasD caused significantly reduced bacterial load (P<0.05) (FIG. 2). Further, mutations in sdrD, isdB, and isdA presented with fewer abscesses when analyzed by macroscopic and microscopic techniques (Table 3). The inventors considered mutants with <30% surface abscesses and <⅓ histological scores to display a significant defect in abscess formation. Mutants with mismatched surface histological scores were not counted as defective; rather, it is attributed to screening errors. Interestingly, mutations in clfB and sasB exhibited defects in staphylococcal load but not in abscess formation, whereas mutations in protein A displayed increased load but also displayed a defect in abscess formation. The inventors wondered whether defects in abscess formation and survival in renal tissue were due to the inability of these mutants to form functional biofilms. In other words, is the defect in abscess formation attributable to defects for in vitro biofilm growth? To study this the inventors cultured staphylococci in 96 well assay plates or on coverslips and measured safranin-stained biofilm by absorbance at 405 nm. S. aureus Newman does not form biofilm in laboratory broth, however Morrissey and colleagues reported biofilm growth in iron-depleted RPMI and 5% atmospheric CO₂ (Johnson et al., 2008). Using these conditions, all mutant strains tested here grew equally well (data not shown), however mutations in srtA, isdB, sdrD or isdA exhibited significant (P<0.05) defects in biofilm formation. These results were corroborated by scanning electron microscopy experiments (FIG. 3B, 3C, 3D, 3E, 3F, 3G, and Table 4), where S. aureus Newman grows as multicellular complexes embedded in a granular extracellular matrix (FIG. 3B). Multicellular complexes are reduced and the extracellular matrix is diminished in staphylococci with mutations in srtA, isdB, or sdrD, the same mutations that also abolished abscess formation in mice. Thus, in vitro biofilm formation may indeed be correlated with the ability of staphylococci to form abscesses. The inventors also tested mutations in genes that abrogate the synthesis of other envelope factors in staphylococci, including poly-N-acetylglucosamine (PNAG/PIA which is synthesized by products of ica genes) (Heilmann et al., 1996; Gotz, 2002) as well as the cell wall associated proteins Eap (Scriba et al., 2008; Xie et al., 2006) and Emp (Hussain et al., 2001). Regulatory factors for staphylococcal virulence were also tested: saeR (Novick and Jiang, 2003), sarA (Cheung et al., 1992), mgrA (Chen et al., 2006), agrC, and agrA (Novick, 2003). Mutation in emp displayed the largest defect in biofilm formation with an 86% reduction in safranin absorbance and the absence of multicellular complexes by scanning EM, results that are in agreement with a similar study of Johnson et al (2008). In contrast, the Eap mutant displayed a slight, but significant defect in biofilm formation (FIG. 3A, Table 4). Emp and Eap are envelope adhesins that mediate interaction between staphylococci and host extracellular matrix proteins fibronectin, vibronectin, and collagen (Scriba et al., 2008; Xie et al., 2006; Hussain et al., 2001). All sequenced strains of S. aureus harbor genes for both proteins, which are positively regulated by the two-component system SaeRS and the global transcription factor SarA, mutations in which also impact biofilm formation (Harraghy et al., 2005) (FIG. 3A, Table 4).

TABLE 4
96 well plate in vitro biofilm assay. Mean absorbance
at 450 nm with standard error, fraction reduction from
Newman absorbance, P-value (student's t-test)

| | Mean absorbance @ | % Reduction | |
|---|---|---|---|
| Strain | 450 nm ± SEM | (0.967-Abs)/0.967 | P-value |
| Newman | 0.967 ± 0.054 | — | — |
| Eap | 0.621 ± 0.152 | 0.357 | 0.011 |
| Emp | 0.131 ± 0.026 | 0.861 | 2.30 × 10−10 |
| SaeR | 0.161 ± 0.025 | | 6.80 × 10−12 |
| SarA | 0.252 ± 0.051 | 0.83 | 1.37 × 10−5 |
| MgrA | 0.630 ± 0.012 | 0.793 | 0.064 |
| AgrC | 0.701 ± 0.121 | 0.319 | 0.388 |
| AgrA | 0.889 ± 0.106 | 0.272 | 0.204 |
| IcaA | 0.516 ± 0.121 | 0.081 | 4.34 × 10−4 |
| IcaC | 0.653 ± 0.204 | 0.466 | 0.041 |
| IcaD | 0.791 ± 0.158 | 0.325 | 0.194 |
| IcaB | 0.713 ± 0.184 | 0.182 | 0.08 |
| IcaR | 0.511 ± 0.169 | 0.472 | 1.19 × 10−3 |
| Ica:tet | 0.546 ± 0.169 | 0.2 | 4.28 × 10−3 |
| SrtA | 0.524 ± 0.081 | 0.458 | 6.51 × 10−5 |
| IsdB | 0.415 ± 0.042 | 0.57 | 8.79 × 10−8 |
| SdrD | 0.447 ± 0.090 | 0.537 | 0.002 |
| IsdA | 0.658 ± 0.913 | 0.32 | 0.029 |
| SasD | 0.679 ± 0.187 | 0.298 | 0.064 |
| SasA | 0.707 ± 0.077 | 0.268 | 0.388 |
| SasG | 0.774 ± 0.184 | 0.2 | 0.204 |
| SasH | 0.777 ± 0.156 | 0.197 | 0.196 |
| SpA | 0.824 ± 0.136 | 0.176 | 0.136 |
| SasC | 0.833 ± 0.087 | 0.147 | 0.39 |
| SasI | 0.797 ± 0.142 | 0.138 | 0.419 |
| SasB | 0.841 ± 0.127 | 0.13 | 0.374 |
| IsdC | 0.863 ± 0.024 | 0.107 | 0.433 |
| FnbA | 0.886 ± 0.368 | 0.084 | 0.677 |
| ClfB | 0.913 ± 0.181 | 0.056 | 0.705 |
| ClfA | 1.061 ± 0.107 | −0.097 | 0.705 |
| SdrE | 1.070 ± 0.124 | −0.107 | 0.465 |
| FnbB | 1.120 ± 0.118 | −0.158 | 0.419 |

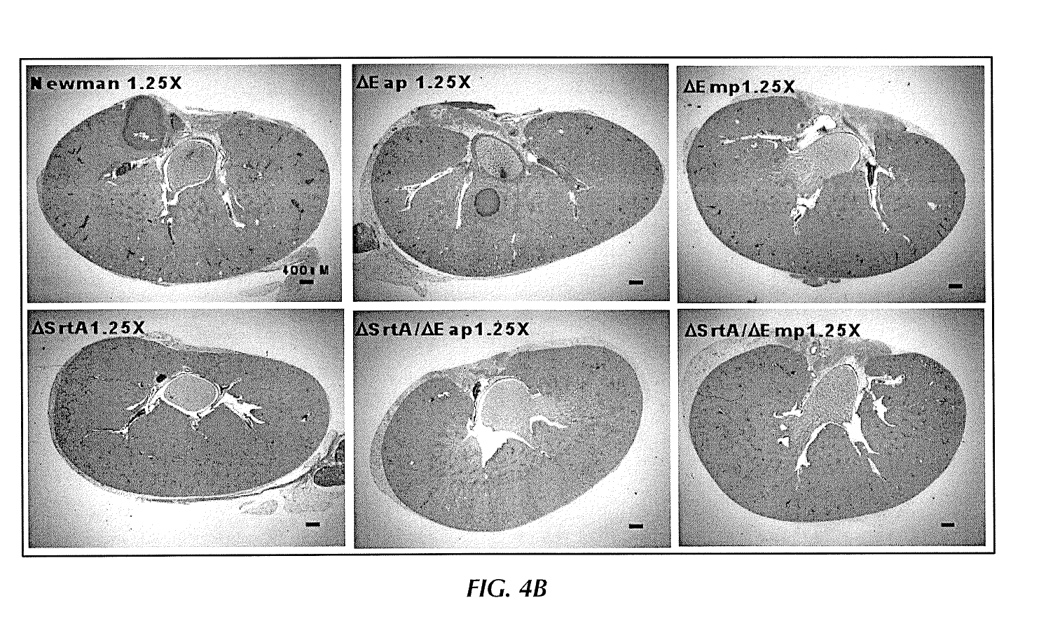

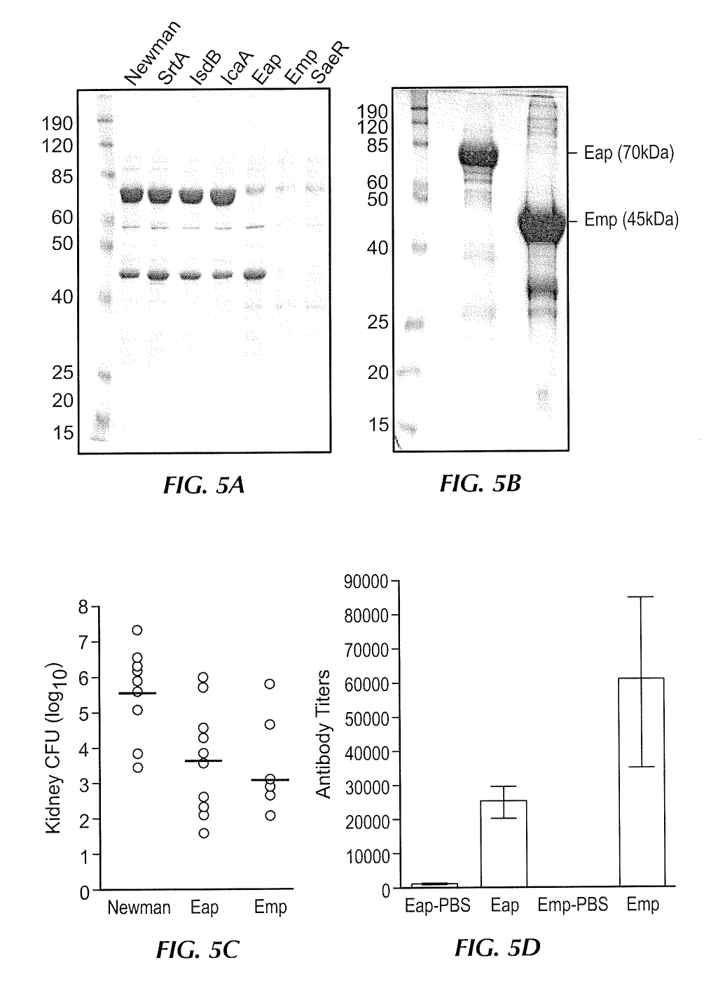

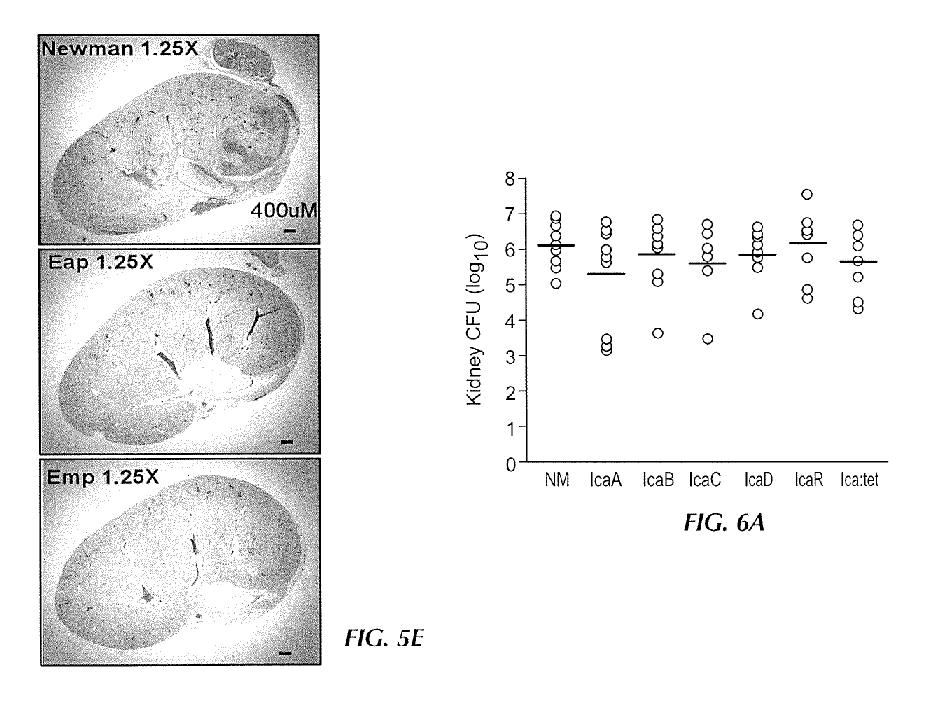

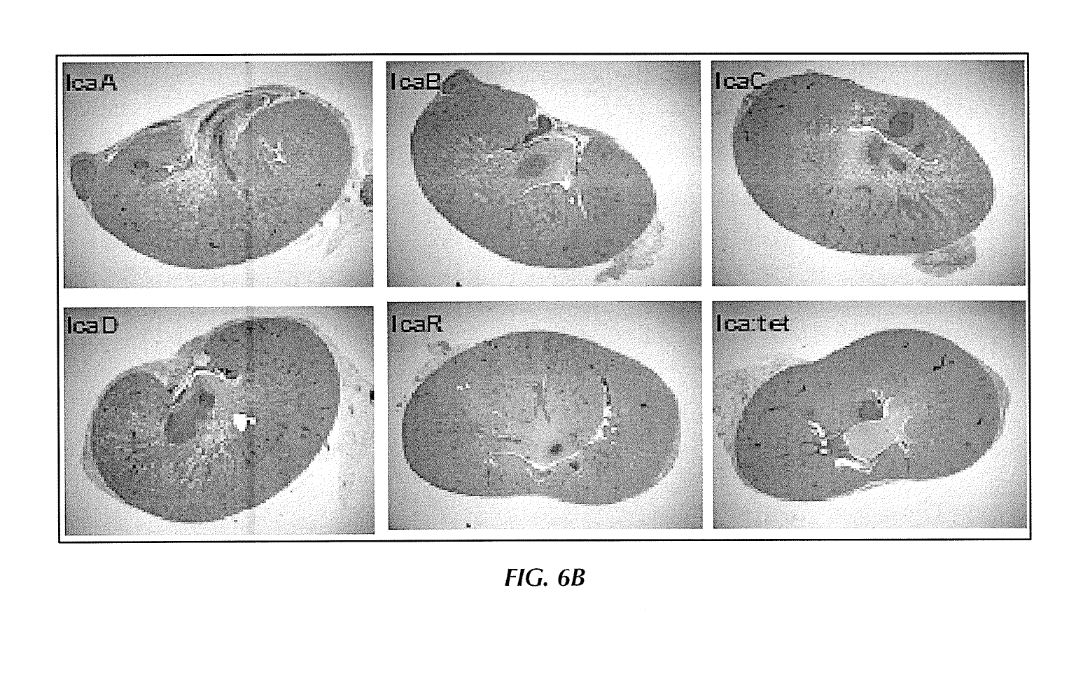

To study if Emp is required for abscess formation, mice were challenged with emp mutant staphylococci and kidneys analyzed five days following infection. The emp mutant staphylococci were isolated from kidney tissue with similar abundance as the wild-type parent (FIG. 4A), however these mutants failed to form abscesses and instead remained dispersed throughout kidney tissue (FIG. 4B). These results identify Emp as an envelope factor that is uniquely required for abscess and biofilm formation in vitro and in vivo. It is noted that mutations in ica genes (icaABCDR), which mediate PNAG synthesis (Heilmann et al., 1997), exhibited only a slight decrease in biofilm formation. Although unable to produce exopolysaccharide, ica mutants were able to generate the extracellular matrix that can be detected by electron microscopy. Further, ica mutants failed to display in vivo defects in abscess formation or bacterial load (FIGS. 6A, 6B, Table 5). These data suggest therefore that the extracellular matrix of S. aureus Newman biofilms is not comprised of PNAG. Emp and Eap are cell wall associated surface proteins, whose production can be detected by SDS extraction of staphylococci and separation on Coomassie-stained PAGE (FIG. 5A). With this assay it was observed that S. aureus Newman produces large quantities of Eap and Emp. Mutations in srtA or isdB do not affect production or cell wall association of Emp and Eap, in agreement with the conjecture that the observed defects of srtA and isdB mutants in abscess and biofilm formation are not due to secondary effects on Eap and Emp. Of note, mutations in emp affect the abundance of Eap and it is surmised that envelope deposition of Emp may affect the surface display of Eap.

TABLE 5
Ica virulence. Mean recovered CFUs, log reduction from Newman, P-value (student's
t-test), % surface abscesses observed, # histological abscesses.

| | Mean S. aureus | | | | |
|---|---|---|---|---|---|
| | per kidneys ± | | | % | |
| | SEM | Reduction | | surface | # histology |
| Strain | (log10(CFU)/mL) | (log10(CFU)/mL) | P-value | abscess | abscess |
| Newman | 6.148 ± 0.194 | — | — | 0.7 | 3 |
| IcaA | 5.326 ± 0.452 | 0.822 | 0.1122 | 0.4 | 2 |
| IcaB | 5.894 ± 0.306 | 0.254 | 0.4917 | 0.35 | 2 |
| IcaC | 5.651 ± 0.441 | 0.497 | 0.3004 | 0.35 | 2 |
| IcaD | 5.886 ± 0.278 | 0.262 | 0.4394 | 0.45 | 2 |
| IcaR | 6.201 ± 0.309 | −0.053 | 0.8837 | 0.6 | 2 |
| Ica:tet | 5.692 ± 0.280 | 0.456 | 0.1909 | 0.55 | 2 |
| SrtA | 3.319 ± 0.604 | 2.849 | 2.26 × 10−4 | 0 | 0 |
| IcaA/SrtA | 2.247 ± 0.559 | 3.901 | 3.45 × 10−6 (N) | 0 | 0 |

1.84 × 10−5 (I)
0.2097 (S)

As both proteins displayed prominently on the staphylococcal surface, the inventors contemplated that Emp and Eap represent suitable vaccine antigens to prevent staphylococcal disease. The structural genes for each protein were cloned into pET15b and recombinant products purified by affinity chromatography via N-terminal His-6 tag under denaturing conditions. Following purification, Eap could be folded and soluble product purified by a second round of Ni-NTA chromatography in renaturing buffer. Purified Emp could not be refolded and was thenceforth kept in 8 M urea (FIG. 5B). Mice were immunized with PBS, Eap or Emp emulsified in complete Freund adjuvant (CFA) and challenged mice with 3×10⁷ CFU S. aureus Newman. FIG. 5C shows that immunization with Emp or Eap conferred significant protection (P<0.05) against staphylococcal infection. Mice vaccinated with Eap displayed a two log reduction in staphylococcal load, whereas Emp immunized mice exhibited a 2.5 log reduction. Mice immunized with Eap (1:24,000) or Emp (>64,000) developed high titers of reactive IgG (FIG. 5D). As expected, animal mock immunized with PBS developed 70% surface and 4/5 histological abscesses. In contrast, mice immunized with Eap and Emp presented with <20% surface abscesses and 1/5 histological lesions (FIG. 5E). Thus, immunization with Eap or Emp generates specific humoral immune responses and protective immunity against staphylococcal infection.

Studies reveal an association between staphylococcal biofilm growth and the ability to form abscesses in infected host tissues. Previous work established that biofilms are required for colonization and persistent infection of implanted medical devices and allow for protection from antibiotics, antibodies, and phagocytic cells. Evidence presented here suggests that biofilms are required for effective seeding and persistent proliferation of staphylococci within organ tissues. Three sortase substrate genes were identified, sdrD, isdB, and isdA that displayed combinatorial defects in abscess formation, staphylococcal load in infected tissues and in vitro biofilm growth. Remarkably, the products of these surface protein genes were also identified as premier vaccine candidates in a comparative evaluation of staphylococcal sortase anchored surface proteins (Stranger-Jones et al., 2006). Such attribute can now also be expanded for two cell wall associated factors Emp and Eap. At least emp is required for abscess formation and biofilm growth and immunization with Emp product affords protective immunity against staphylococcal disease. Thus, these studies expand the list of suitable vaccine candidates to prevent human infections with S. aureus to select cell wall anchored and cell wall associated proteins, whose combined formulation should provide strong protective immunity against all staphylococcal strains.

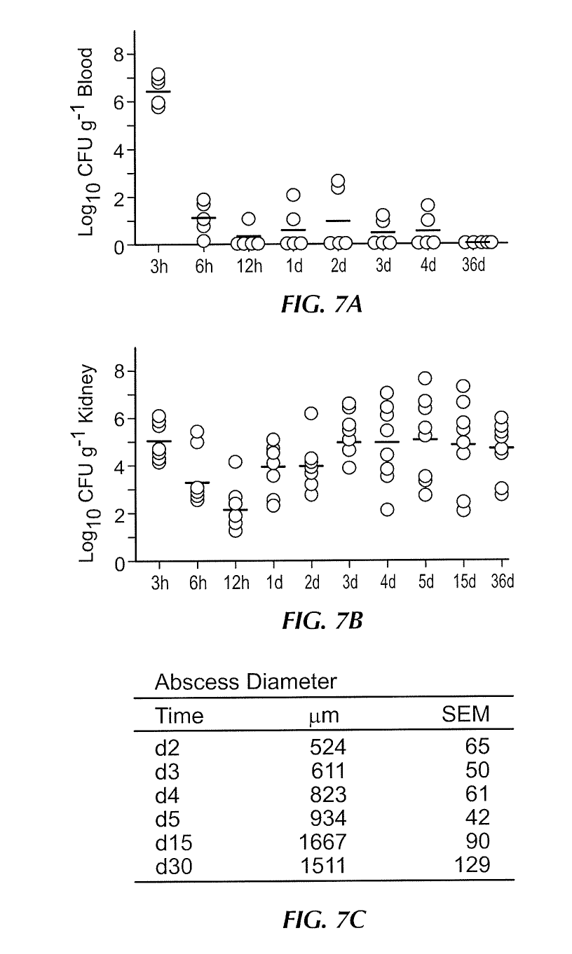

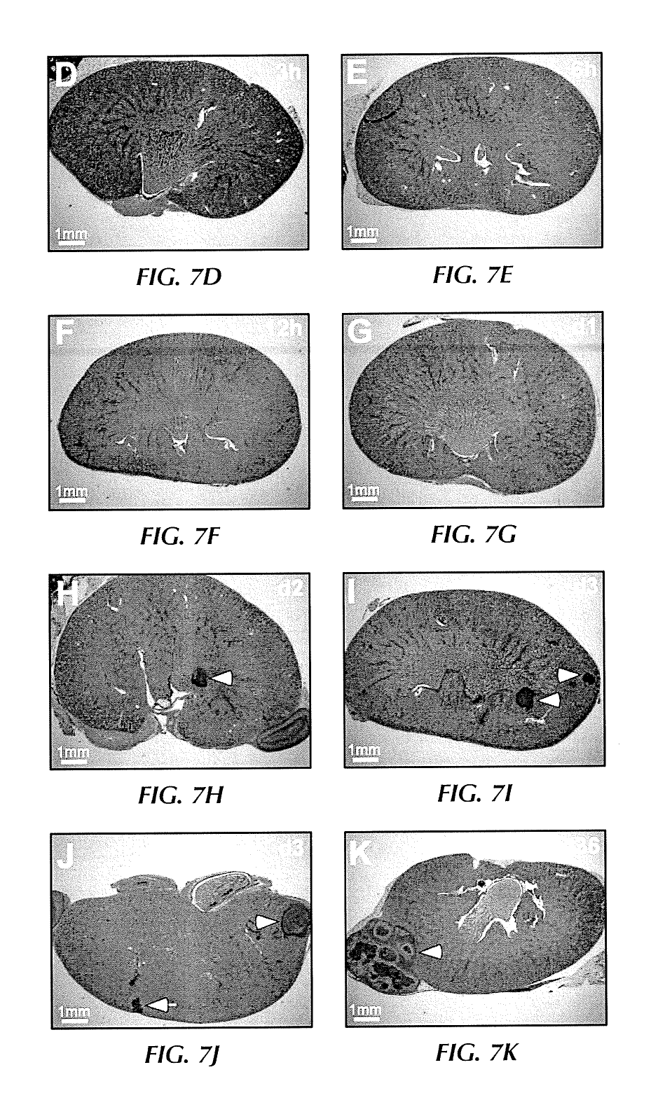

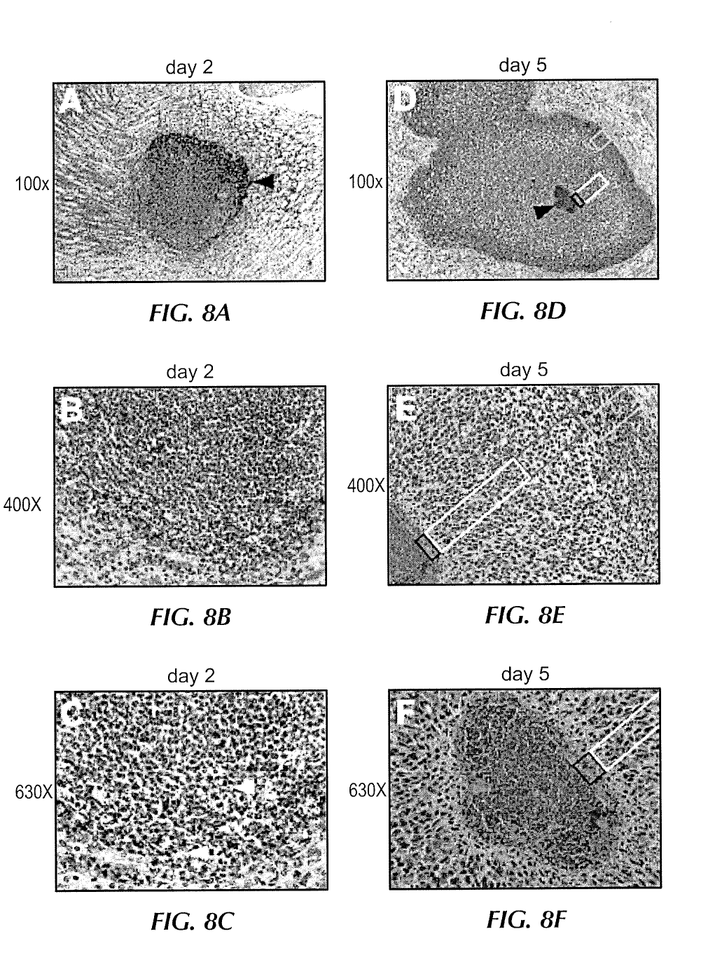

Animal model for staphylococcal abscess formation and persistent infection. To characterize the pathogenesis of S. aureus abscess formation, the renal abscess model was modified (Albus et al., 1991), wherein BALB/c mice were infected by intravenous injection with 1×10⁷ CFU of the human clinical isolate S. aureus Newman (Baba et al., 2007). Within 6 hours following infection, 99.999% of staphylococci disappeared from the blood stream and were distributed via the vasculature (FIG. 7A). Staphylococcal dissemination to peripheral tissues occurred rapidly, as the bacterial load in kidney and other peripheral organ tissues reached 1×10⁵ CFU g⁻¹ within the first three hours (FIG. 7B). The staphylococcal load in kidney tissues increased by 1.5 log CFU within twenty-four hours (FIG. 7B). Forty-eight hours following infection, mice developed disseminated abscesses in multiple organs, detectable by light microscopy of hematoxylin-eosin stained, thin-sectioned kidney tissue (FIG. 7D-K). The initial abscess diameter was 524 μM (±65 μM); lesions were initially marked by an influx of polymorphonuclear leukocytes (PMNs) and harbored no discernable organization of staphylococci, most of which appeared to reside within PMNs (FIG. 8A-C). On day 5 of infection, abscesses had increased in size and enclosed a central population of staphylococci, surrounded by a layer of eosinophilic, amorphous material and a large cuff of PMNs (FIG. 8D-F). Histopathology revealed massive necrosis of PMNs in proximity to the staphylococcal nidus at the center of abscess lesions as well as a mantle of healthy phagocytes (FIG. 8D-F). A rim of necrotic PMNs at the periphery of abscess lesions, bordering eosinophilic, amorphous material that separated healthy renal tissue from the infected lesion were also observed (FIG. 8D-F). Abscesses eventually reached a diameter of ≧1,524 μM on day 15 or 36 (FIG. 7K). At later time intervals, staphylococcal load was increased to 10⁴-10⁶ CFU g⁻¹ and growing abscess lesions migrated towards the organ's capsule (FIG. 7J-K). Peripheral lesions were prone to rupture, thereby releasing necrotic material and staphylococci into the peritoneal cavity or the retroperitoneal space. These events resulted in bacteremia as well as a secondary wave of abscesses, eventually precipitating a lethal outcome of these infections (data not shown).

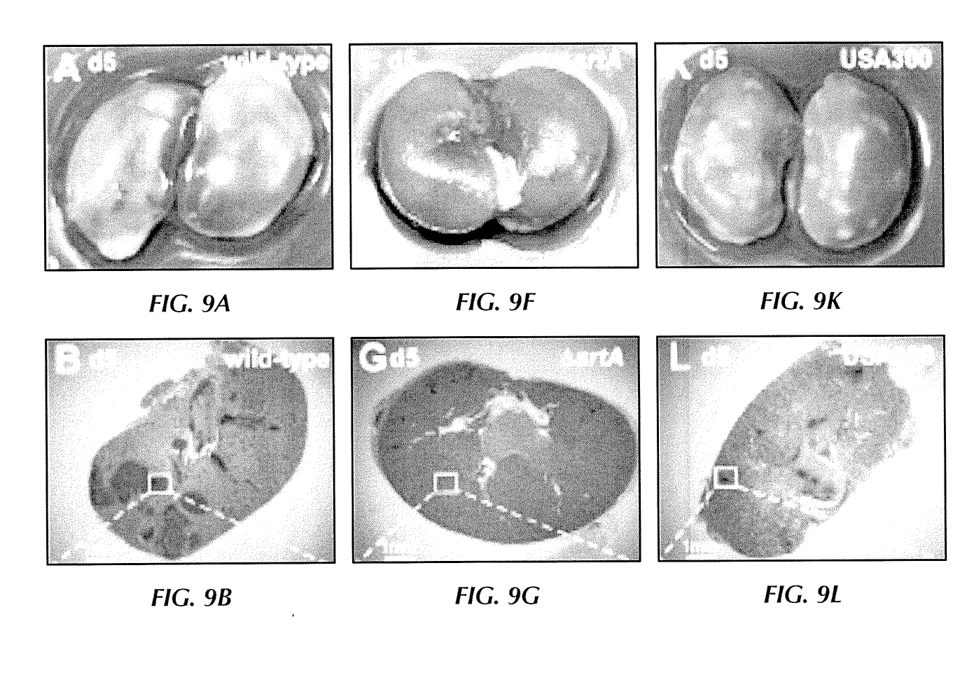

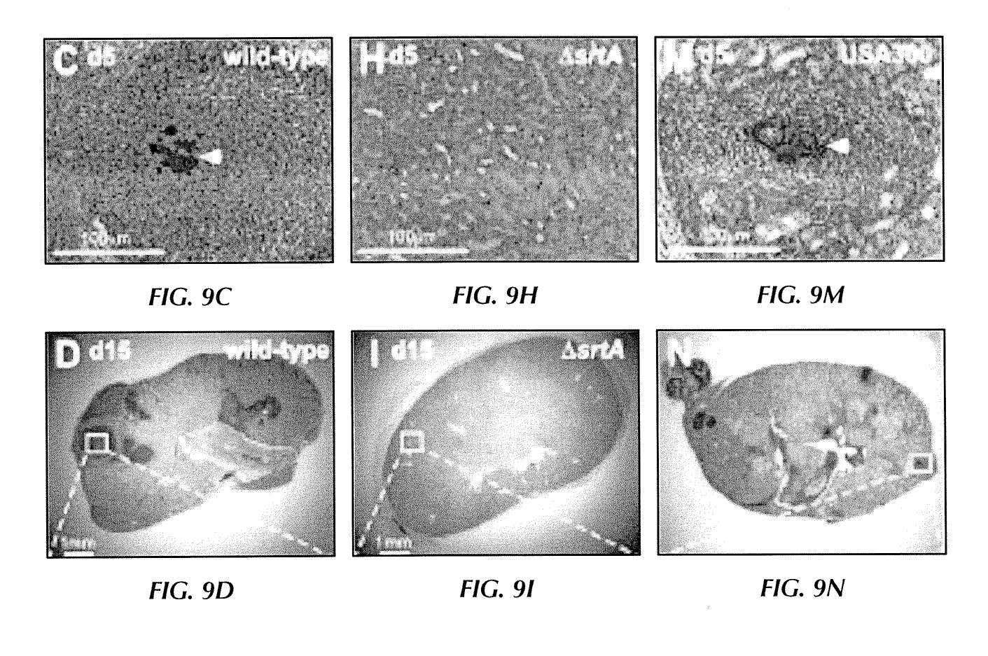

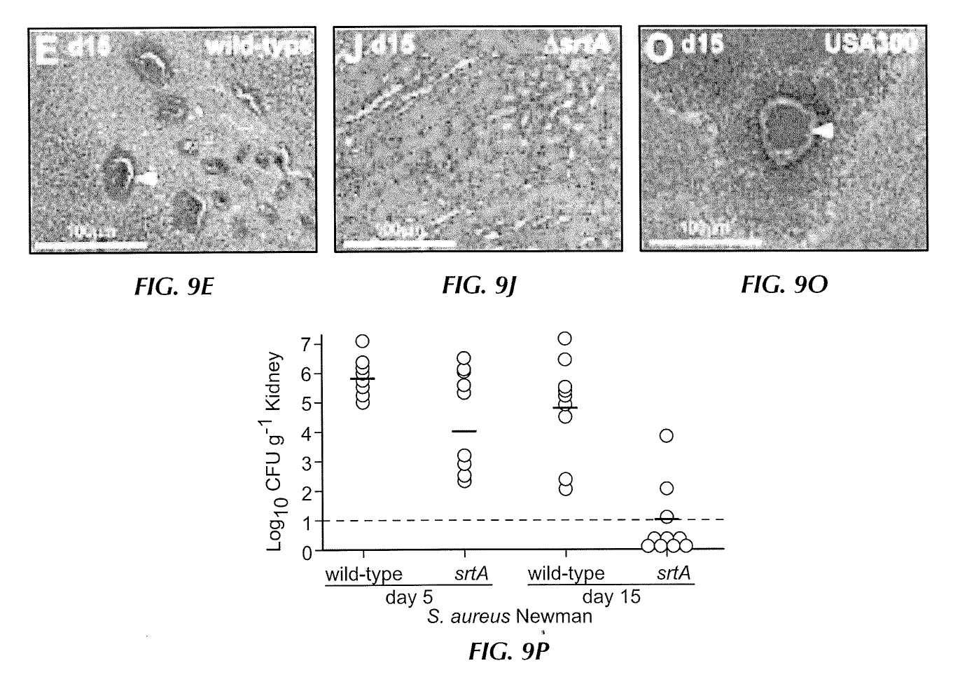

Staphylococcal abscess communities are enclosed by a pseudocapsule. To enumerate staphylococcal load in renal tissue, animals were killed, their kidneys excised and tissue homogenate spread on agar media for colony formation. On day 5 of infection, a mean of 1×10⁶ CFU g⁻¹ renal tissue for S. aureus Newman was observed (FIG. 9P). To quantify abscess formation, kidneys were visually inspected, and each individual organ was given a score of one (FIG. 9A) or zero (FIG. 9F). The final sum was divided by the total number of kidneys to calculate percent surface abscesses (Table 6). In addition, randomly chosen kidneys were fixed in formalin, embedded, thin sectioned, and stained with hematoxylin and eosin. For each kidney, four sagittal sections at 200 μM intervals were viewed by microscopy (FIG. 9). The numbers of lesions were counted for each section and averaged to quantify the number of abscesses within kidneys. S. aureus Newman caused 4.364±0.889 abscesses per kidney, and surface abscesses were observed on 14 out of 20 kidneys (70%) (Table 6).

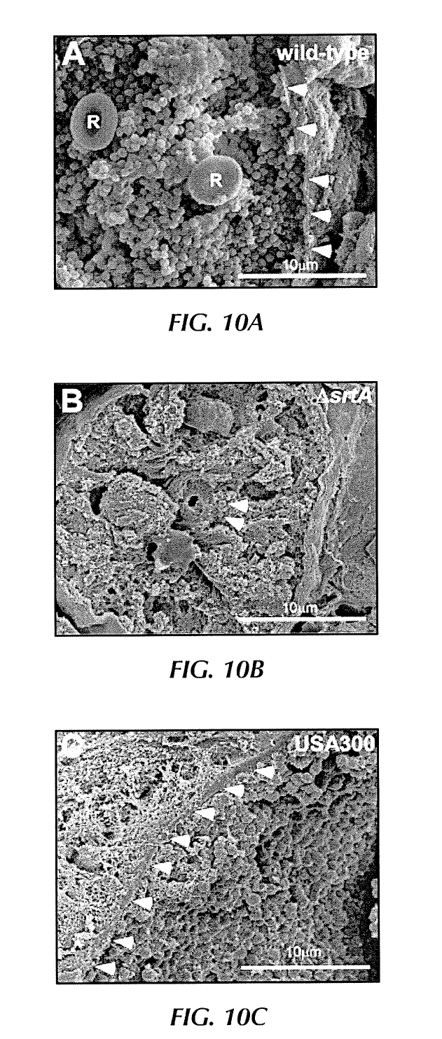

Kidneys were sectioned, fixed, dehydrated and sputter coated with platinum/palladium for scanning electron microscopy. FIG. 10A shows S. aureus Newman in tightly associated lawns at the center of abscesses. Staphylococci were contained by an amorphous pseudocapsule (white arrow heads, FIG. 10A) that separated bacteria from the cuff of abscesses leukocytes. No immune cells were observed in these central nests of staphylococci, however occasional red blood cells were located among the bacteria (R, FIG. 10A). Bacterial populations at the abscess center, designated staphylococcal abscess communities (SAC), appeared homogenous and were coated by an electron-dense, granular material. The kinetics of the appearance of infectious lesions and the morphological attributes of abscesses formed by S. aureus Newman were similar to those observed following mouse infection with S. aureus USA300 (LAC), the current epidemic community acquired methicillin-resistant S. aureus (CA-MRSA) clone in the United States (Diep et al., 2006) (FIG. 9K-O and 10C).

TABLE 6
Genetic requirements for S. aureus Newman abscess formation in mice.

| Staphylococcal load in kidney tissue | | | | Abscess formation in kidney tissue | | |
|---|---|---|---|---|---|---|
| | ^{a}log₁₀ CFU g⁻¹ | ^{b}Significance | ^{c}Reduction | ^{d}Surface | ^{e}Number of | ^{f}Significance |
| Genotype | tissue | (P-value) | (log₁₀ CFU g⁻¹) | abscesses (%) | abscesses per kidney | (P-value) |
| wild-type | 6.141 ± 0.192 | — | — | 70 | 4.364 ± 0.889 | — |
| ΔsrtA | 4.095 ± 0.347 | 6.7 × 10⁻⁶ | 2.046 | 0 | 0.000 ± 0.000 | 0.0216 |

Surface protein genes

| | | | | | | |
|---|---|---|---|---|---|---|
| sdrD | 4.092 ± 0.454 | 0.0001 | 2.049 | 15 | 0.600 ± 0.267 | 0.0265 |
| isdB | 4.535 ± 0.298 | 5.7 × 10⁻⁵ | 1.606 | 5 | 0.500 ± 0.167 | 0.0227 |
| clfB | 4.672 ± 0.302 | 0.0001 | 1.469 | 30 | 1.852 ± 0.654 | 0.1298 |
| isdA | 4.723 ± 0.299 | 0.0002 | 1.418 | 15 | 0.375 ± 0.182 | 0.0350 |
| isdC | 5.050 ± 0.208 | 0.0004 | 1.091 | 27 | 1.000 ± 0.327 | 0.0737 |
| clfA | 5.103 ± 0.260 | 0.0025 | 1.038 | 40 | 1.125 ± 0.350 | 0.0848 |
| spa | 5.137 ± 0.374 | 0.0144 | 1.004 | 13 | 0.375 ± 0.374 | 0.0356 |
| sasG | 5.139 ± 0.287 | 0.0054 | 1.002 | 45 | 1.222 ± 0.425 | 0.0770 |
| sasC | 5.193 ± 0.337 | 0.0167 | 0.948 | 56 | 1.375 ± 0.595 | 0.1335 |
| sasD | 5.312 ± 0.291 | 0.0212 | 0.829 | 48 | 1.500 ± 0.462 | 0.1272 |
| sasA | 5.355 ± 0.217 | 0.0102 | 0.786 | 39 | 2.250 ± 0.453 | 0.2568 |
| sdrE | 5.498 ± 0.255 | 0.0475 | 0.643 | 65 | 2.333 ± 0.667 | 0.5023 |
| sasF | 5.518 ± 0.318 | 0.0884 | 0.623 | 47 | 1.333 ± 0.408 | 0.3187 |
| isdH | 5.555 ± 0.251 | 0.0676 | 0.586 | 44 | 1.125 ± 0.479 | 0.0859 |
| sasB | 5.650 ± 0.255 | 0.1641 | 0.491 | 59 | 1.720 ± 0.620 | 0.1651 |
| fnbA | 5.678 ± 0.270 | 0.1294 | 0.463 | 51 | 2.125 ± 0.666 | 0.2338 |
| sdrC | 5.693 ± 0.287 | 0.1908 | 0.448 | 33 | 1.000 ± 0.378 | 0.0741 |
| fnbB | 5.823 ± 0.246 | 0.3124 | 0.318 | 54 | 2.000 ± 0.567 | 0.2074 |

PNAG (PIA) genes

| | | | | | | |
|---|---|---|---|---|---|---|
| icaA | 5.326 ± 0.452 | 0.3122 | 0.815 | 40 | 2.667 ± 1.453 | 0.5768 |
| icaB | 5.894 ± 0.306 | 0.4917 | 0.247 | 35 | 1.000 ± 0.270 | 0.2690 |
| icaC | 5.651 ± 0.441 | 0.3004 | 0.491 | 35 | 2.000 ± 1.527 | 0.4384 |
| icaD | 5.886 ± 0.278 | 0.4394 | 0.255 | 45 | 1.667 ± 0.667 | 0.3741 |
| icaR | 6.201 ± 0.309 | 0.8837 | +0.06 | 60 | 2.333 ± 0.333 | 0.5033 |
| ica:tet | 5.692 ± 0.280 | 0.1909 | 0.449 | 55 | 2.333 ± 0.667 | 0.5023 |

Envelope associated protein genes

| | | | | | | |
|---|---|---|---|---|---|---|
| eap | 6.530 ± 0.385 | 0.1217 | +0.49 | 55 | 1.250 ± 0.412 | 0.0971 |
| emp | 5.540 ± 0.040 | 0.0576 | 0.601 | 20 | 0.800 ± 0.416 | 0.0361 |

Capsular polysaccharide genes

| | | | | | | |
|---|---|---|---|---|---|---|
| capO | 6.028 ± 0.579 | 0.9825 | 0.113 | 50 | 3.000 ± 1.054 | 0.6035 |

^{a}Means of staphylococcal load calculated as log₁₀ CFU g⁻¹ in homogenized renal tissues 5 days following infection in cohorts of fifteen BALB/c mice per challenge strain. Standard error of the means (±SEM) is indicated.
^{b}Statistical significance was calculated with the Students t-test and P-values recorded; P-values <0.05 were deemed significant.
^{c}Reduction in bacterial load calculated as log₁₀ CFU g⁻¹.
^{d}Abscess formation in kidney tissues five days following infection was measured by macroscopic inspection (% positive)
^{e}Histopathology of hematoxylene-eosin stained, thin sectioned kidneys from eight to ten animals: the average number of abscesses per kidney was recorded and averaged again for the final mean (±SEM).
^{f}Statistical significance was calculated with the Students t-test and P-values recorded, P-values <0.05 were deemed significant.

Sortase mutants cannot establish abscess lesions and fail to persist. Sortase A is a transpeptidase that immobilizes nineteen surface proteins in the envelope of S. aureus strain Newman (Mazmanian et al., 1999; Mazmanian et al., 2000). Earlier work identified sortase A as a virulence factor in multiple animal model systems, however the contributions of this enzyme and its anchored surface proteins to abscess formation or persistence have not yet been revealed (Jonsson et al. 2002; Weiss et al., 2004). Compared to the wild-type parent (Baba et al., 2007), an isogenic srtA variant (ΔsrtA) failed to form abscess lesions on either macroscopic or histopathology examination on days 2, 5 or 15 (FIG. 9F-J and Table 6). In mice infected with the strA mutant, only 1×10⁴ CFU g⁻¹ was recovered from kidney tissue on day 5 of infection, which is a 2.046 log₁₀CFU g⁻¹ reduction compared to the wild-type parent strain (P=6.73×10⁻⁶)(FIG. 9P). A similar defect was observed for the srtA mutant of MRSA strain USA300 (data not shown). Scanning electron microscopy showed that srtA mutants (arrow heads) were highly dispersed and often associated with leukocytes in otherwise healthy renal tissue (FIG. 10B). On day fifteen following infection, srtA mutants were cleared from renal tissues, a ≧3.5 log₁₀CFU g₋₁ reduction compared to the wild-type (FIG. 9P). Thus, sortase A anchored surface proteins enable the formation of abscess lesions and the persistence of bacteria in host tissues, wherein staphylococci replicate as communities embedded in an extracellular matrix and shielded from surrounding leukocytes by an amorphous pseudocapsule.

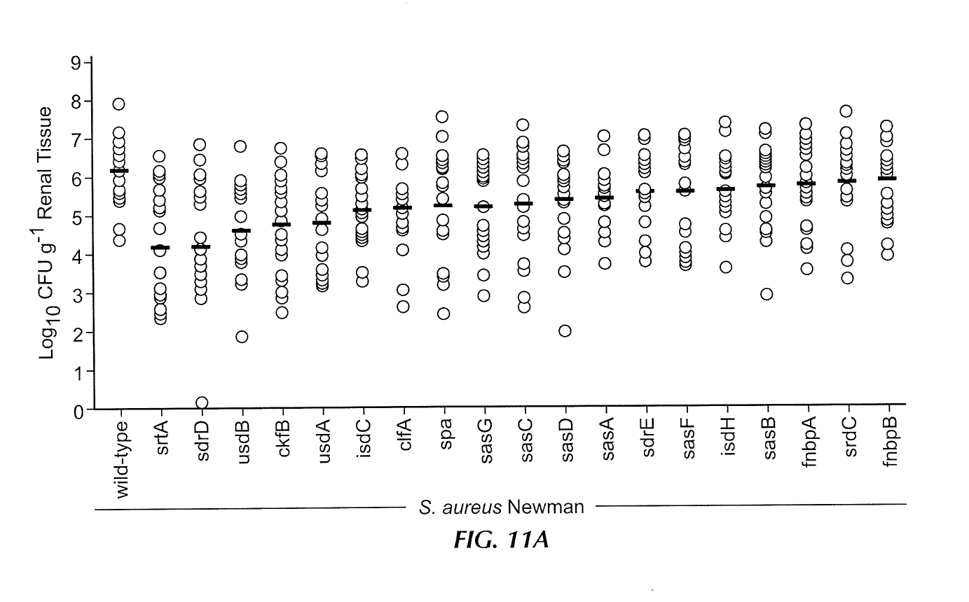

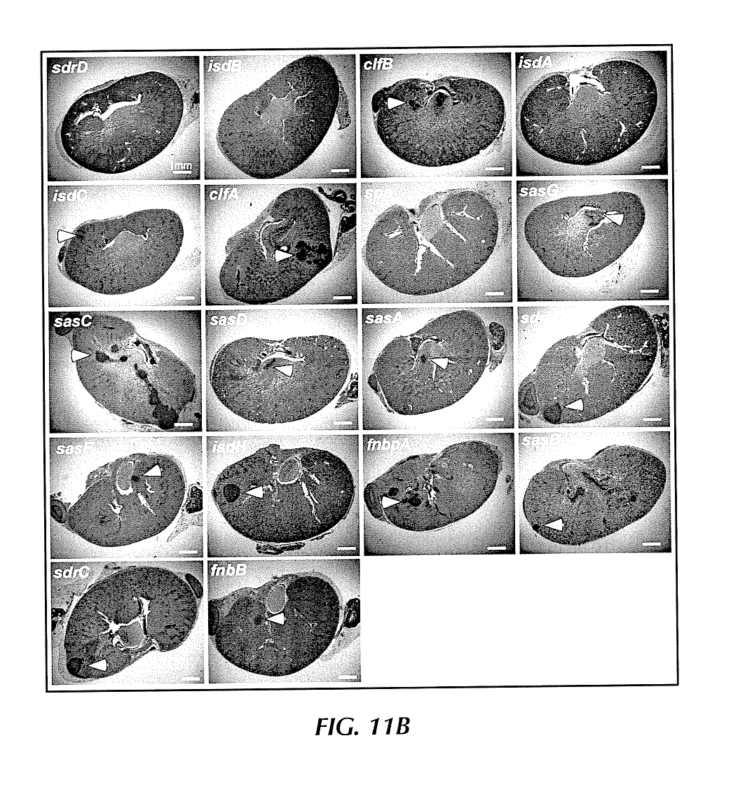

Genetic requirements for staphylococcal surface proteins. Sortase A anchors a large spectrum of proteins with LPXTG motif sorting signals to the cell wall envelope, thereby providing for the surface display of many virulence factors (Mazmanian et al., 2002). To identify surface proteins required for staphylococcal abscess formation, bursa aurealis insertions were introduced in 5′ coding sequences of genes that encode polypeptides with LPXTG motif proteins (Bae et al., 2004) and transduced these mutations into S. aureus Newman. Following intravenous infection of mice and analysis through the renal abscess model, the severity of observed virulence defects was rank ordered as the log₁₀ reduction of the means of staphylococcal CFU g⁻¹ (FIG. 11 and Table 6). Mutations in sdrD, isdB, clfB, isdA, clfA, and isdC caused reduced bacterial load (Table 6). The inventors considered mutants <30% or less surface abscesses and histology abscess average P<0.05 as significant for defects in abscess formation, which included variants with mutations in sdrD, isdB, and isdA (Table 6). Interestingly, mutations in clfA and clfB exhibited defects in staphylococcal load but not in abscess formation (FIG. 11). These virulence findings are in agreement with previous studies suggesting that clumping factor proteins mediate fibrinogen binding as well as resistance to phagocytic clearance, attributes required for pathogen survival and dissemination in blood (McDevitt et al., 1994; Ni Eidhin et al. 1998). Protein A impedes phagocytosis by binding the Fc component of immunoglobulin (Uhlen et al., 1984; Jensen et al., 1958), activates platelet aggregation via the von Willebrand factor (Hartleib et al., 2000), functions as a B cell superantigen by capturing the Fab region of VH3 bearing IgM (Roben et al., 1995), and, through its activation of TNFR1, can initiate staphylococcal pneumonia (Gomez et al., 2004). Protein A mutants (spa) exhibited a modest reduction in staphylococcal load (day 5), however, in contrast to wildtype, clfA and clfB strains, the ability of spa variants to form abscesses was diminished (FIG. 11 and Table 6).

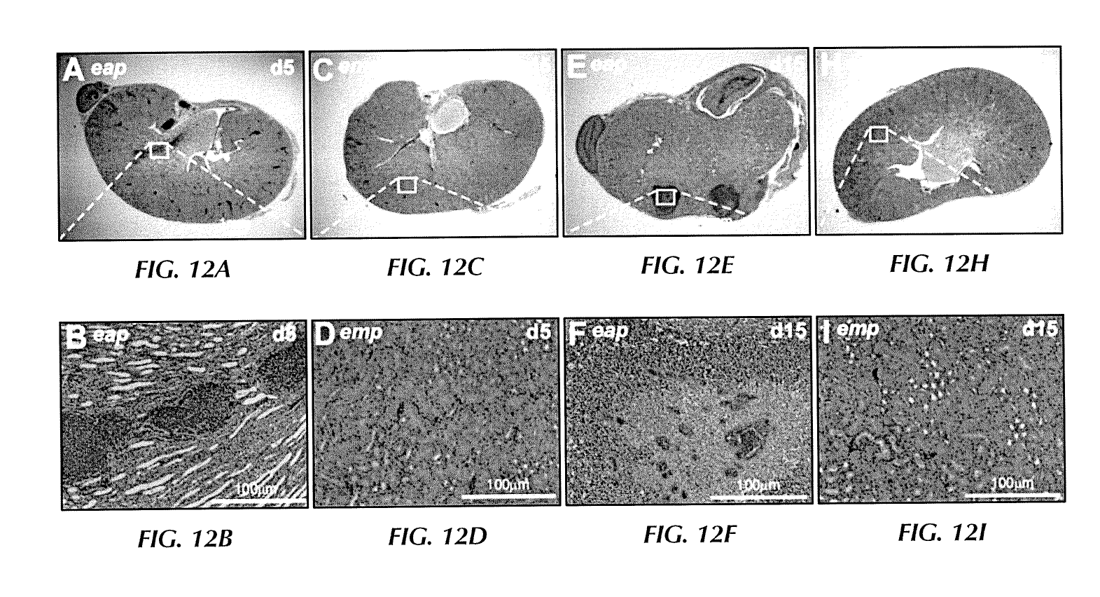

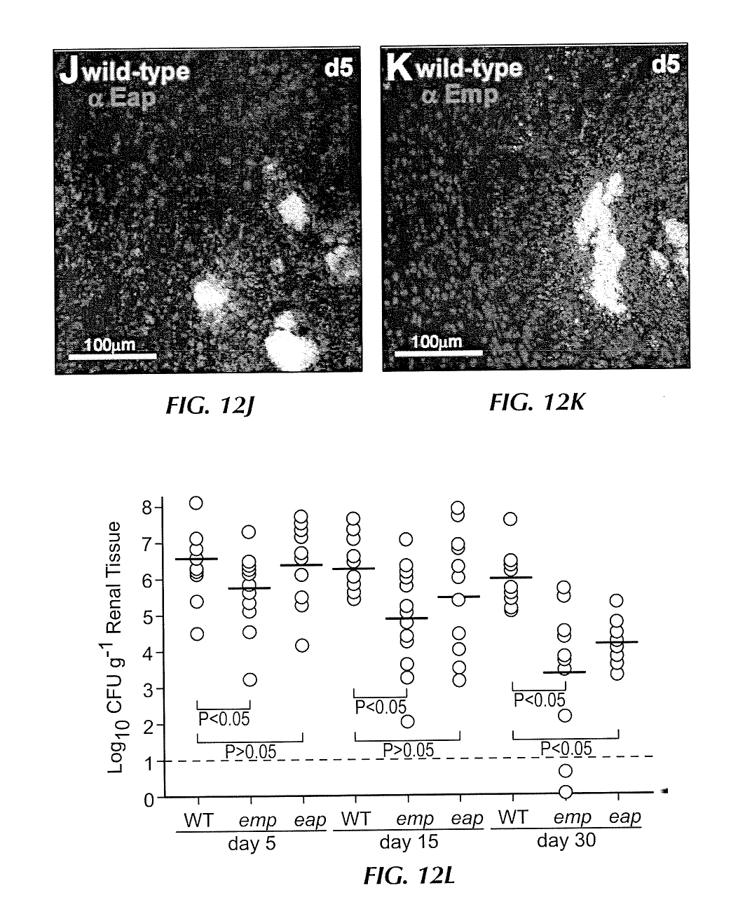

Staphylococcal carbohydrates and envelope associated proteins. S. aureus elaborates two carbohydrate structures, capsular polysaccharide (CPS) (Jones 2005) and poly-N-acetylglucosamine (PNAG) (Gotz 2002). S. aureus Newman and USA300 synthesize type 5 CPS, which is composed of a repeating trisaccharide subunit [→4)-β-D-ManAcA-(1→4)-α-L-FucNAc(3OAc)-(1→3)-β-D-FucNAc-(1→] (Baba et al., 2007). Nucleotide sequences of the cap5 gene cluster comprise a 16 gene operon (capA-P) and two of its products, CapP and CapO, function as epimerase and dehydrogenase in the synthesis UDP-N-acetylmannosaminuronic acid (UDP-ManNAcA) (O'Riordan and Lee, 2004; Sau et al., 1997). As expected, bursa aurealis insertion into cap0 abrogated CPS5 synthesis (data not shown). PNAG (or PIA), a linear β(1-6)-linked glucosaminoglycan, is composed of 2-deoxy-2-amino-D-glucopyranosyl residues, of which 80-85% are N9 acetylated (Mack et al., 1996); the remaining glucosamine residues are positively charged and promote association of the polysaccharide with the bacterial envelope (Vuong et al., 2004). PNAG is synthesized by products of the intercellular adhesin locus (icaADBC) (Heilmann et al., 1996; Cramton et al., 1999). Both S. aureus carbohydrate structures were dispensable for the pathogenesis of animal infections, as mutations in capO as well as icaADBC or the regulator icaR did not affect bacterial load on day 5, the establishment of staphylococcal communities or renal abscess formation (Table 6). The contribution of envelope associated proteins to staphylococcal abscess formation was also examined. The hallmark of envelope associated proteins is that they can be extracted by boiling in hot SDS. This method was used to detect the deposition of two such proteins, Eap and Emp, in the envelope of S. aureus Newman. A mutant with bursa aurealis insertion in emp displayed reduced bacterial load in kidney tissue on day 5 of infection in addition to significant defects in the formation of abscesses and in bacterial persistence within host tissues (FIG. 12, Table 6). No reduction in abscess formation was observed for the eap mutant, whereas the reduced staphylococcal load on day 5 and 15 suggests a defect in bacterial peristence within host tissues (FIG. 12, Table 6). Expression of Emp and Eap during infection was detected with immunofluorescence experiments (FIG. 12J-K). Eap was found deposited within the pseudocapsule, whereas Emp was detected in staphylococal abscess communities. These observations support a model whereby Emp contributes to the formation of staphylococcal communities that elicit abscess lesions, whereas Eap deposition in the pseudocapsule promotes bacterial persistence in host tissues.

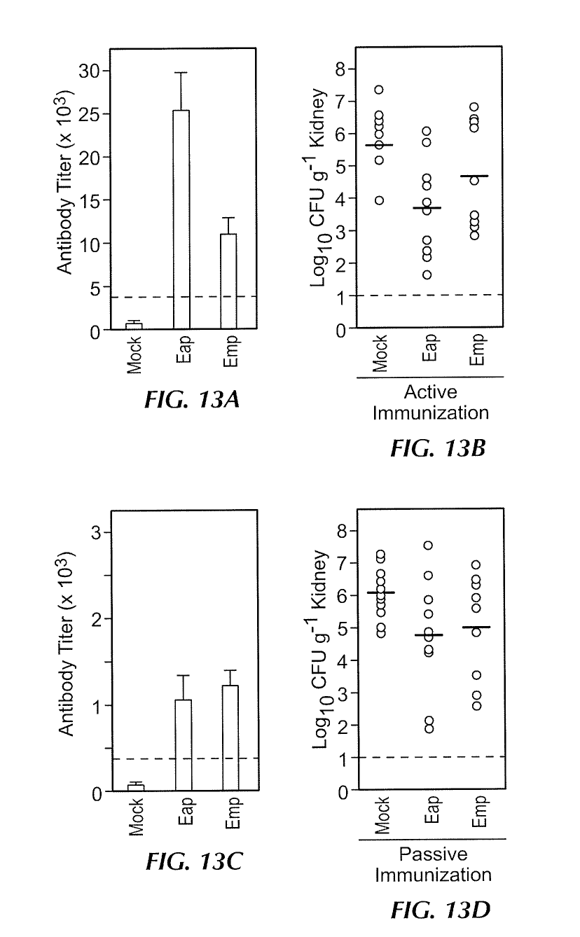

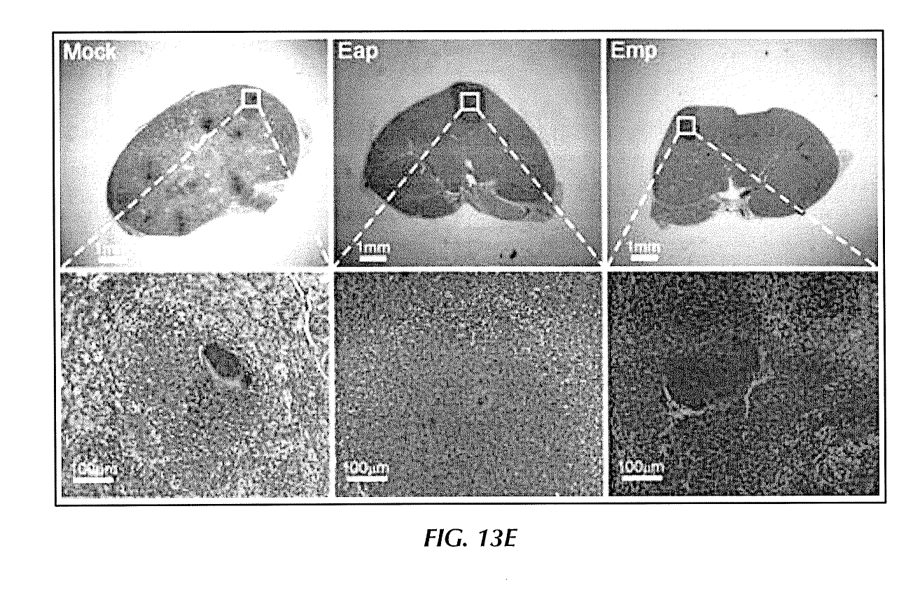

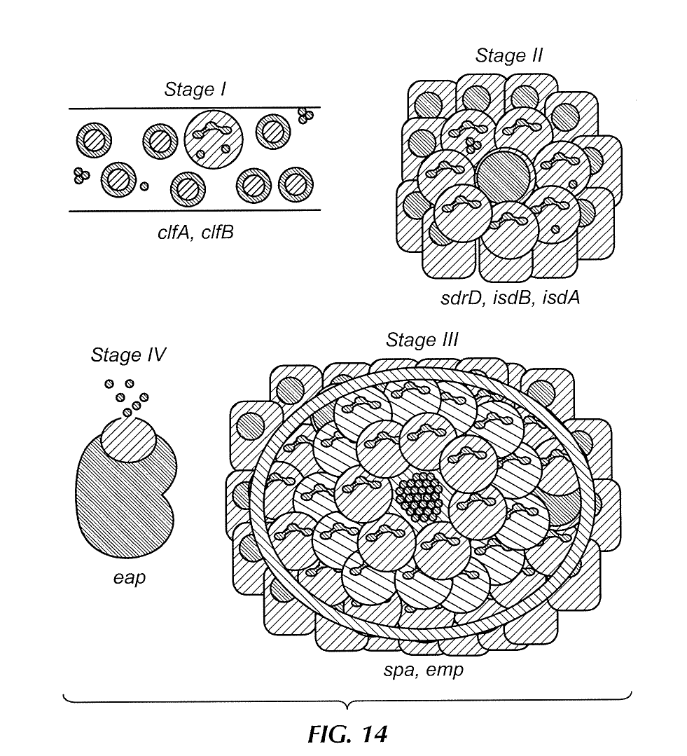

Envelope associated proteins as vaccine antigens. Previous work sought to characterize S. aureus vaccine antigens by interrogating purified sortase A substrates for their ability to elicit protective immunity towards staphylococcal disease (Stranger-Jones et al., 2006). When used as individual subunit vaccine antigens, surface proteins generated variable degrees of protection; immunization with SdrD, IsdA, IsdB, SdrE, SpA, ClfA as well as ClfB achieved a significant reduction in bacterial load, however none of these vaccines afforded complete protection. In contrast, a combination of four antigens generated much more robust vaccine protection against abscess formation or lethal challenge with several different S. aureus strains (Stranger-Jones et al., 2006). Recombinant Eap and Emp was purified from Escherichia coli and used these proteins to immunize BALB/c mice for subsequent challenge with S. aureus Newman (FIG. 13). Following immunization, mice developed humoral immune responses against both envelope associated proteins (FIG. 13A). Immunization with Emp caused a modest 0.959 log₁₀ CFU g⁻¹ reduction in staphylococcal load within kidney tissues (P=0.5114), whereas a significant level of protection was achieved with Eap (1.939 log₁₀CFU g⁻¹ reduction in bacterial load, P=0.0079) (FIG. 13B). To test whether Emp or Eap specific antibodies can provide protection against staphylococcal challenge, rabbits were immunized and Emp-as well as Eap-specific antibodies were purified by affinity chromatography.

Passive immunization with 5 mg kg⁻¹ (85 μg per animal) purified antibodies into the peritoneal cavity of naïve BALB/c mice resulted in low, but detectable levels of serum IgG 24 hours following transfer (antibody titers of 1,000±110 for Eap and 1,124±236 for Emp, FIG. 13C). In parallel, passively immunized animals were challenged by intravenous inoculation with S. aureus Newman, which, when compared to mock controls, resulted in a 1.36 log₁₀CFU g⁻¹ reduction in staphylococcal load for Eap immunized animals (n=10) on day 4 (P=0.0085) and a reduction in the number of abscesses formed (mock treated 4.64±1.09 abscesses kidney⁻¹ vs. Eap immunized 1.40±0.48, P=0.028, n=14 and 10, FIG. 13D-E).

Animals (n=9) that received Emp-specific antibodies displayed a 1.20 log₁₀CFU g⁻¹ reduction in staphylococcal load on day 4 (P=0.0132), but only a slightly reduced number of abscesses formed (2.0±0.98, P=0.1362, FIG. 13D-E). In summary, similar to sortase anchored surface proteins, antibodies against envelope associated factors can generate protection against staphylococcal infection in mice.

### B. Materials and Methods

Bacterial Strains and Growth. Staphylococci were cultured on tryptic soy agar or broth at 37° C. E. coli strains DH5a and BL21(DE3) were cultured on Luria agar or broth at 37° C. Ampicillin (100 μg/ml) and erythromycin (10 μg/ml) were used for plasmid and transposon mutant selection, respectively.

Transposon Mutagenesis. Insertional mutations from the Phoenix library were transduced into human clinical isolate S. aureus Newman. Each mutant carries the transposon bursa aurealis containing an erythromycin resistance cassette in the gene of interest. The mutations were verified as previously described (Bae et al., 2004). Briefly, chromosomal DNA was extracted (Promega Wizard Kit), digested with Acil (NEB), religated with T4 Ligase (Promega) to form individual plasmids, and PCR amplified using primers specific to the transposon bursa aurealis. These products were sequenced to verify the site of transposon insertion in the target gene.

Cloning, purification, and antibody generation. Coding sequences for Eap and Emp were PCR-amplified using S. aureus Newman template DNA (Baba et al., 2007). PCR products were cloned into pET15b to express recombinant proteins with an N-terminal His6 tag fusion. Bacteria were disrupted in a French press, membrane and insoluble components sedimented by ultracentrifugation. His-tagged Emp was purified by affinity chromatography in its native state. Extract containing Eap was solubilized at room temperature in 8 M urea, 50 mM Tris-HCl pH 8.0 for 4-5 hours, then centrifuged at 10,000×g. The supernatant containing the denatured protein was subjected to nickel-nitrilotriacetic acid (Ni-NTA) affinity chromatography (Promega). Protein was eluted in PBS-8M urea containing successively higher concentrations of imidazole (100-500 mM). Eluate fractions positive for Eap were pooled, diluted into PBS-1M Urea and passed over a second Ni-NTA column. Refolded Eap was eluted with PBS buffer containing imidazole. Protein concentration was determined by absorbance at 280 nm. Rabbits (6 month old New-Zealand white, females, Charles River Laboratories) were immunized with 500 μg protein emulsified in CFA (Difco) for initial immunization or IFA for booster immunizations on day 24 and 48. On day 60, rabbits were bled and serum recovered for immunoblotting, immune-fluorescence microscopy or passive transfer experiments.

Scanning Electron Microscopy. Infected kidneys (right side) were fixed for 24-48 hours in 8% glutaraldehyde at 4° C. and sectioned into 2-5 mm pieces to expose internal tissues or abscesses. These samples were then dehydrated by successive incubations in 25, 50, 75, 90, 100% ethanol, followed by 100% HMDS. Following dehydration, samples were mounted and sputter coated with 80% Pt/20% Pd to 8 nm before viewing under a Fei NovaNano SEM200 scanning electron microscope. For biofilm assays, staphylococci were grown on coverslips in iron depleted RPMI in 5% CO₂ and washed 3 times in PBS. Cover slips were serially dehydrated by incubation in ethanol and HMDS, mounted, and sputter coated prior to viewing under the scanning electron microscope.

Biofilm formation. S. aureus strains were grown in Chelex (Sigma) treated RPMI 1640 (Gibco) supplemented with 10% RPMI 1640 and 1% Casamino acids (Difco). Overnight cultures were grown at 37° C. in 6% CO₂, then inoculated 1:10 in quadruplicate into 96-well flat-bottomed tissue culture plates (Costar) containing fresh media. These plates were incubated statically at 37° C. in 6% CO₂ for 24 hours. Wells were washed three times with 1×PBS, dried for 2 hours at 37° C., and stained with 1% safranin. Absorbance at 450 nm was measured to quantify biofilm formation. Each strain was tested in at least 3 separate experiments and a two-tailed Student t test was used to compare mutants to wild-type.

Renal Abscess. Overnight cultures of S. aureus Newman were inoculated 1:100 into fresh tryptic soy broth and grown for 2 hours at 37° C. Staphylococci were sedimented, washed with 1×PBS, and suspended in a volume of PBS to yield an A600 of 0.6 (3×10⁸ CFU/ml). The inoculum was verified by plating and colony enumeration. Mice were anesthetized by intraperitoneal injection of 100 mg/ml of ketamine and 2 mg/ml of xylazine per kilogram of body weight. 6-8 week old female BALB/c mice (Charles River Laboratories) were infected with 100 μl of bacterial suspension (3×10⁷ CFU) by retroorbital injection. Cohorts of 10 or 20 mice were infected per staphylococcal strain. On the day 5 following infection, mice were killed by CO₂ inhalation, dissected, and the kidneys were excised and homogenized in 0.01% Triton X-100 using a sonicator. Aliquots (20 μl) were serially diluted and plated for determination of CFU. Three to four right kidneys from each cohort of mice were fixed in 10% formalin for 24 h at room temperature. Tissues were embedded in paraffin, thin-sectioned, stained with hematoxylin and eosin, and examined by microscopy. 3-4 week old female BALB/c mice were used for persistence studies.

Immunization. BALB/c mice (24-day-old female, 8-10 mice per group, Charles River Laboratories, Wilmington, Mass.) were immunized by intramuscular injection into the hind leg with purified protein. Antigen (25 μg purified protein per animal) was administered on days 0 (emulsified 1:1 with complete Freund's adjuvant) and 11 (emulsified 1:1 with incomplete Freund's adjuvant). Mice were bled periorbitally on day 20, followed by retro-orbital challenge in the opposite eye with 10⁷ CFU/ml bacteria on day 21. Mice were killed on day 25 and processed according to the renal abscess model.

Immunofluorescence microscopy. Kidneys of infected animals were dissected, placed in 1×PBS on ice, and then flash frozen in Tissue Tek OCT Compound within cryomolds. Samples were thin sliced (4 μm thick), mounted on slides, and stored at −80° C. Prior to staining, slides were warmed to room temperature for 30 minutes, fixed in ice cold acetone for 10 minutes, and washed twice with ice cold PBS. The slides were blocked in 3% BSA, 1:20 Human IgG (Sigma), 1×PBS, 0.1% Tween-80 for 1 hour at room temperature with shaking Specific rabbit antibody (1:2,000) was added to the mixture and slides were allowed to incubate for another hour. The solution was decanted and glass slides were washed 3 times with PBS and 10 minute incubations each. Slides were placed in 3% BSA, 1×PBS, 0.1% Tween-80, 1:200 AlexaFluor-647 mouse anti-rabbit secondary antibody and allowed to incubate at room temperature in the dark, with shaking. The solution was decanted, slides were washed 3 times with PBS, placed in PBS containing 1:1,000 Hoechst dye (Invitrogen) as well as 1 μg/ml BODIPY-vancomycin and allowed to incubate in the dark for 5 minutes with shaking The slides were washed once more with PBS, mounted in Npropylgallate, and viewed under a Leica SP5 AOBS spectral two-photon confocal microscope.

Active and passive immunization. BALB/c mice (n=15) were immunized with purified Eap or Emp or PBS on day 0, 11. On day 20 following immunization, 5 mice were bled to obtain sera to determine antibody titers and on day 21, all mice were challenged with 1×10⁷ CFU S. aureus Newman. Five days following infection, kidneys were removed during necropsy and renal tissue analyzed for staphylococcal load or histopathology.

Rabbit Eap or Emp antibodies were purified by affinity chromatography (purified Eap or Emp covalently linked to sepharose) and transferred by intraperitoneal injection into mice. Passively immunized animals were challenged twenty-four hours later by retroorbital injection with 1×10⁷ CFU S. aureus Newman. Serum IgG titers of actively or passively immunized animals were analyzed by ELISA. Four days following infection, kidneys were removed during necropsy and renal tissue was analyzed for staphylococcal load or histopathology.

### Example 2

### Staphylococcus Aureus Synthesizes Adenosine to Escape Host Immune Responses

### A. Results

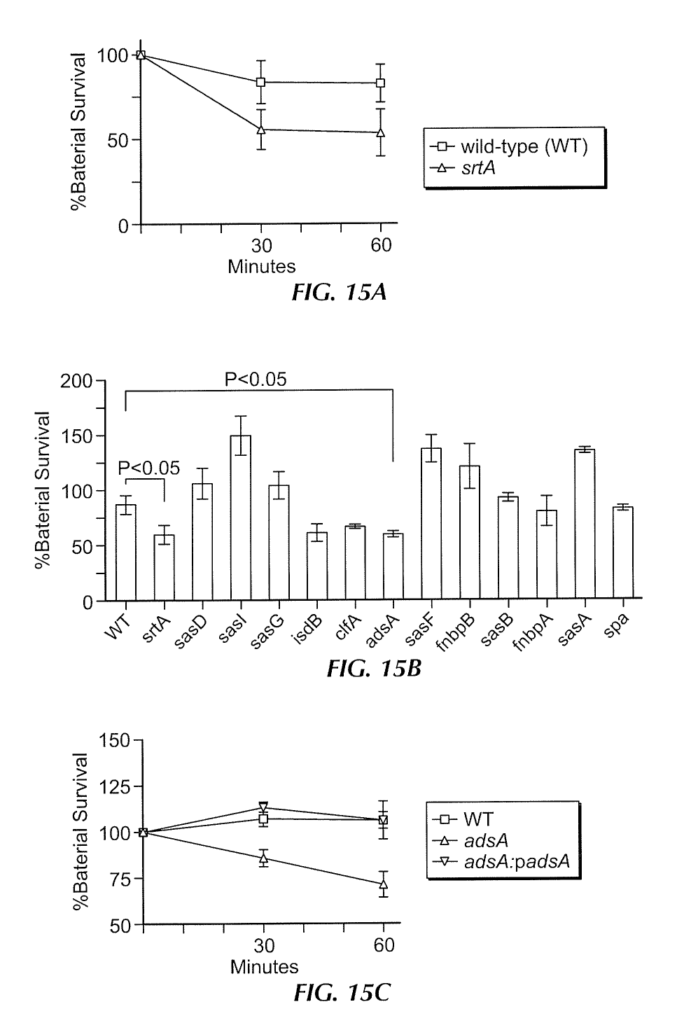

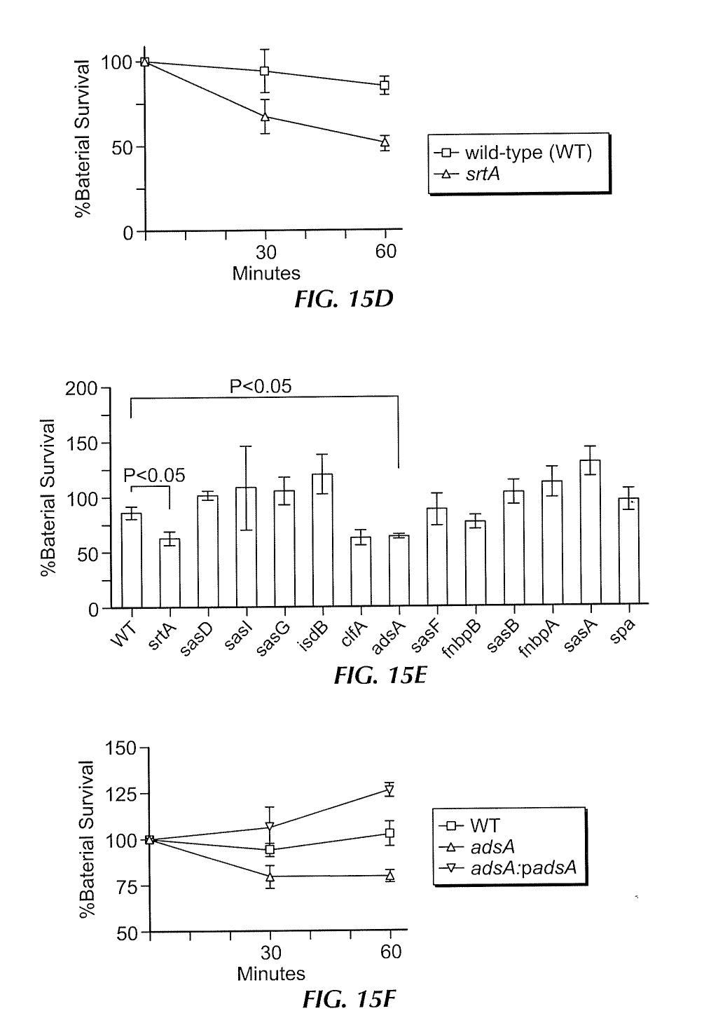

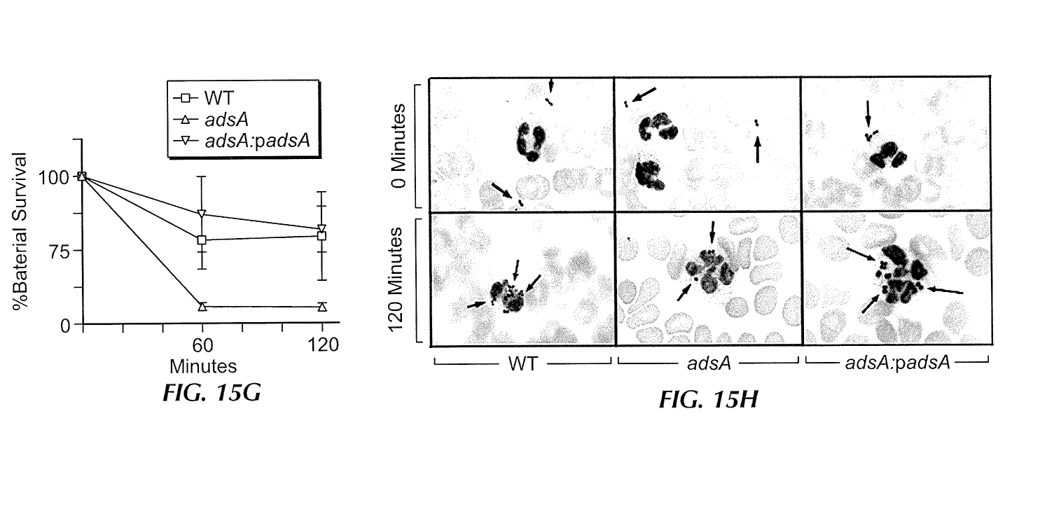

AdsA is required for staphylococcal survival in blood. To identify staphylococcal genes required for escape from innate immune responses, the ability of S. aureus strain Newman to survive in whole blood from BALB/c mice or Sprague-Dawley rats was examined by recording bacterial load at timed intervals via the formation of colonies on agar medium (FIG. 15). As expected, immune cells within blood of naïve mice and rats, which lack antibodies specific for staphylococci (data not shown), were unable to phagocytose and kill S. aureus Newman (FIGS. 15A and 15D). In contrast to the wild-type strain, a variant lacking the structural gene for sortase A (srtA) displayed a defect in staphylococcal escape from phagocytic killing (P<0.05) (FIGS. 15A and 15D). Sortase A anchors a large spectrum of different polypeptides in the staphylococcal envelope, using a transpeptidation mechanism and LPXTG motif sorting signal at the C-terminus of surface proteins (Mazmanian et al., 2002). To examine these surface proteins for their contribution to staphylococcal escape from phagocytic killing, the inventors transduced bursa aurealis insertions in surface protein genes (Bae et al., 2004) into wild-type strain S. aureus Newman and measured survival of staphylococcal variants in blood (FIGS. 15B and 15E). Mutations in clfA and sasH (Staphylococcus aureus surface protein), hereafter named adsA, displayed consistent survival defects. The phenotype of clfA mutants represents an expected result, as the encoded clumping factor A (ClfA) product is known to precipitate fibrin and interfere with macrophage and neutrophil phagocytosis (Palmqvist et al., 2004; Higgins et al., 2006). The contribution of AdsA to pathogenesis is not yet known. AdsA harbors a 5′-nucleotidase domain with the two signature sequences ILHTnDiHGrL (residues 124-134) and YdamaVGNHEFD (residues 189-201), suggesting that the protein may catalyze the synthesis of adenosine from 5′-AMP. To further examine the importance of adsA in staphylococcal virulence, the inventors complemented the adsA gene by cloning the entire adsA gene and upstream promoter sequences into expression vector pOS1, generating padsA. Transformation of adsA mutant staphylococci with padsA restored their ability to survive in mouse or rat blood, indicating that the observed virulence defect is indeed caused by the absence of adsA expression (FIGS. 15C and 15F, and FIG. 20). S. aureus survival was also examined in blood of human volunteers. As with murine blood, the number of adsA mutant staphylococci was reduced and staphylococcal phagocytosis by neutrophils was increased as compared to wild-type strain S. aureus Newman (FIGS. 15G and 15H).

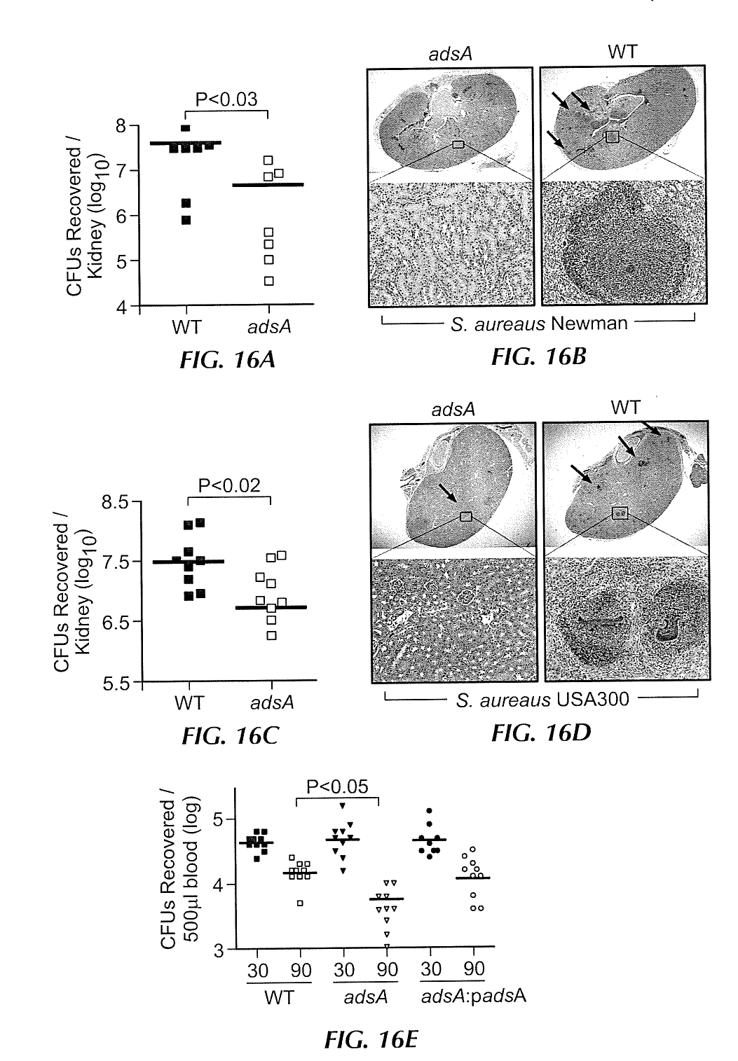

AdsA is required for staphylococci virulence and abscess formation. To investigate the contribution of adsA in invasive staphylococcal disease, BALB/c mice were infected by intravenous inoculation with 10⁷ colony forming units (CFU) of wild type S. aureus Newman or its isogenic asdA variant. Animals were killed 5 days post-infection and both kidneys were removed. The right kidney was homogenized and staphylococcal load enumerated by plating on agar and colony formation (FIG. 16A). The left kidney was fixed with glutaraldehyde, embedded in paraffin, thin sectioned and analyzed by histology (FIG. 16B). As expected, wild-type S. aureus Newman formed abscesses in kidney tissue with an average bacterial load of 10⁷ CFU per gram of organ tissue. In contrast, adsA mutant staphylococci were unable to form abscesses and displayed a greater than ten-fold reduction in bacterial load, as compared to the wild-type (FIG. 16A).

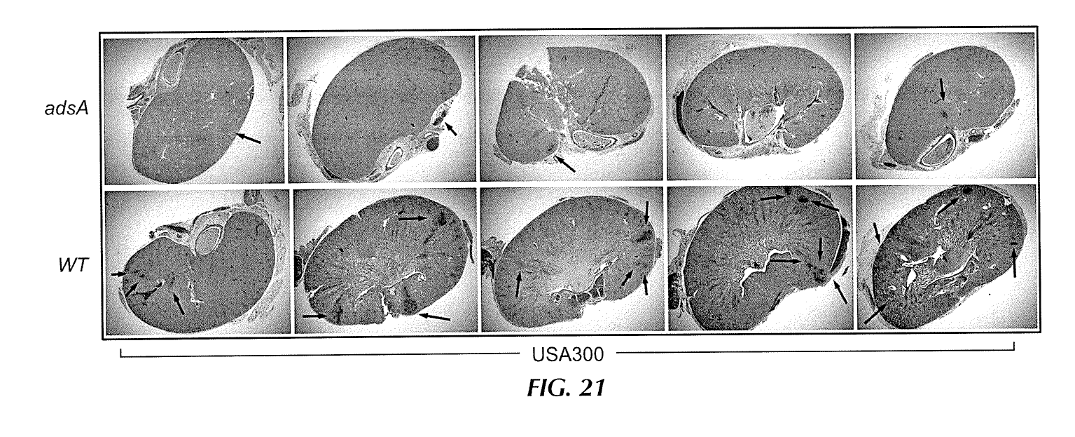

Infections with MRSA strains that were acquired in communities of the United States (CA-MRSA) have been characterized by pulsed-field gel electrophoresis and DNA sequencing. Currently, the major CA-MRSA clone is USA300 (McDougal et al., 2003), the predominant cause of skin and soft tissue infections as well as bacteremia (Carleton et al., 2004). To assess the contribution of adsA towards virulence of USA300, an isogenic adsA mutant was isolated using phage transduction and S. aureus strain LAC (USA300) (Bae et al., 2004). BALB/c mice were infected by retro-orbital injection of staphylococci into the blood stream. Five days following challenge, staphylococci were enumerated in homogenized kidney tissue and the histopathologies of abscesses were visualized in hematoxylin-eosin stained thin sections of this organ (FIG. 16C). Similar to S. aureus Newman, the inventors observed a one-log reduction in CFU recovered from the kidneys of animals infected with the adsA mutant of S. aureus USA300. Further, fewer abscesses and smaller lesions were observed in kidneys of mice infected with the adsA variant (FIG. 16D and FIG. 21). Together these results document the requirement of adsA for virulence in two clinical isolates, S. aureus strains Newman and USA300.

Differences in abscess formation and recovery of CFUs from kidneys of infected mice may stem from enhanced bacterial clearance in the blood stream, causing fewer bacteria to reach peripheral organ tissues. Alternatively, adsA could play a direct role in the formation of abscesses and infectious foci. To discern between these possibilities, BALB/c mice were infected by retro-orbital inoculation and peripheral blood was sampled at timed intervals by cardiac puncture. In agreement with observations of enhanced clearance of adsA mutant staphylococci in vitro, significantly fewer CFU of adsA mutant staphylococci were retrieved 90 minutes post-infection, as compared to the wild-type parent strain S. aureus Newman. Further, transformation of the adsA mutant strain with padsA restored its ability to survive in blood following intravenous challenge (FIG. 16E). Although we cannot rule out the possibility that adsA contributes also specifically to abscess formation, these data suggest that the reduced virulence of adsA mutant staphylococci results from their decreased survival in blood.

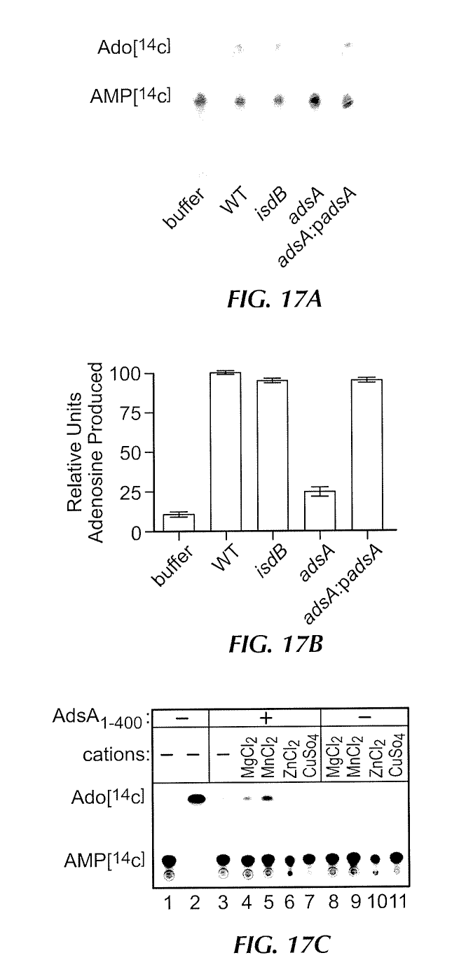

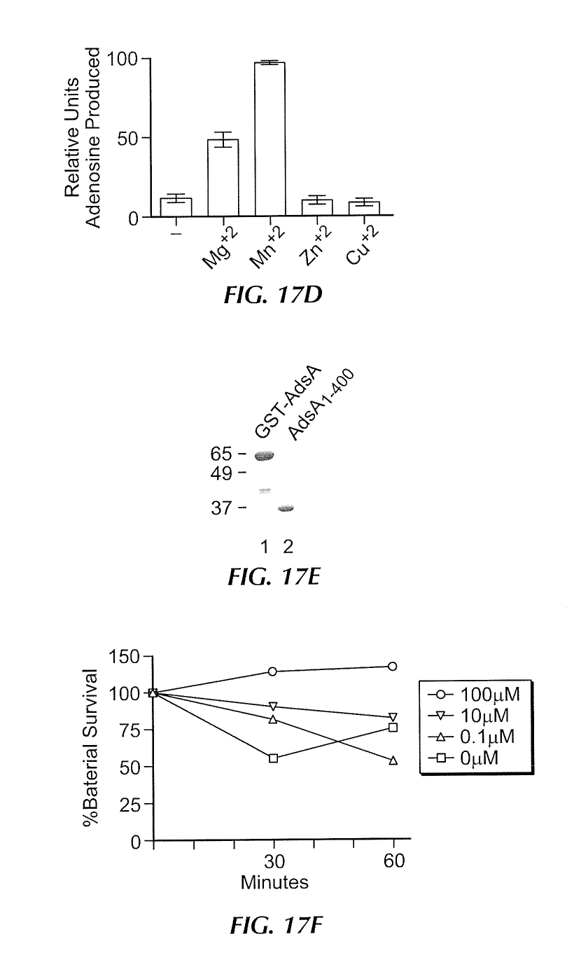

AdsA-mediated synthesis of adenosine correlates with staphylococcal survival in blood. Given that AdsA harbors a 5′nucleotidase signature sequence, it was asked whether AdsA can synthesize adenosine from AMP. Cell wall peptidoglycan of S. aureus wild-type, adsA and isdB (iron surface determinant B, a gene that does not contribute to AMP hydrolysis) (Mazmanian et al., 2003) mutant strains, and the adsA:padsA strain was degraded with lysostaphin (Schindler and Schuhardt, 1964), and cell wall extracts were incubated with radiolabeled [¹⁴C]AMP. Production of adenosine was monitored by thin layer chromatography (TLC). Lysostaphin extracts of adsA mutant staphylococci displayed significantly reduced adenosine synthase activity (˜25% of wild-type). Adenosine synthase activity was restored to wild-type levels when adsA mutants were transformed with padsA (FIG. 17A). Disruption of isdB, in contrast, did not affect the generation of adenosine by S. aureus.

To characterize the enzymatic activity of AdsA, we expressed and purified a soluble recombinant form of AdsA from Escherichia coli. Purified AdsA cleaved [¹⁴C]AMP to generate adenosine and maximal activity (K_{M}=44 nM) was observed in the presence of 5 mM MgCl₂ or 5 mM MnCl₂, similar to the metal requirements of other adenosine synthases (Zimmermann, 1992). On the other hand, incubation of AdsA with 5 mM ZnCl₂ or 5 mM CuSO₄ prior to the addition of [¹⁴C]AMP, completely inhibited adenosine synthase activity (FIG. 17B, lanes 6 and 7). A similar inhibiting effect was observed when EDTA, a divalent metal ion chelator, was added to AdsA, demonstrating that AdsA requires divalent cations for adenosine synthase activity in vitro.

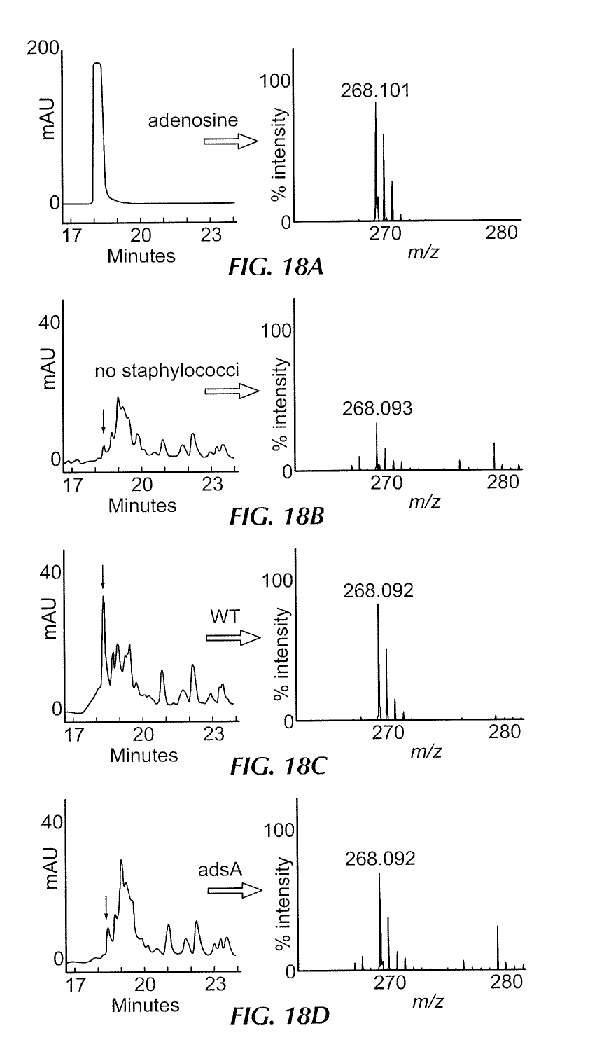

It was contemplated that staphylococci escape phagocytic clearance in blood by synthesizing adenosine. The survival defect of adsA mutant staphylococci in blood could be rescued by exogenous supplies of adenosine. This was tested, revealing a dose-dependent increase in the survival of adsA mutant staphylococci upon the addition of 1-100 μM adenosine (FIG. 17F). Under physiological conditions, the concentration of AMP in the extracellular milieu is estimated to be in the nanomolar range. Immunological insult or tissue injury, however, causes release of AMP whose concentration may increase up to 100 μM. It therefore seems plausible that these AMP stores may be converted to adenosine during staphylococcal infection. To assess the relative abundance of adenosine during staphylococcal infection, mouse blood was infected with S. aureus for 60 min. Plasma was retrieved, protein removed and samples subjected to reverse phase high pressure liquid chromatography (rpHPLC). For calibration, commercially purified adenosine was separated by rpHPLC and determined its molecular mass in the eluate (FIG. 18A). Chromatography of uninfected blood revealed the adenosine absorption peak, whose identity was confirmed by mass spectrometry (FIG. 18A). The adenosine peak in blood was increased ten-fold following infection with S. aureus Newman (FIG. 18C), whereas infection with the isogenic adsA mutant produced less than a two-fold increase in adenosine (FIG. 18D). Of note, extracellular adenosine is imported rapidly by blood cells (half life <1 min) (Thiel et al., 2003). In view of this, the observed ten-fold increase of adenosine in blood during S. aureus infection represents a substantial accumulation of this signaling molecule and an important virulence strategy whereby staphylococci combat host immunity.

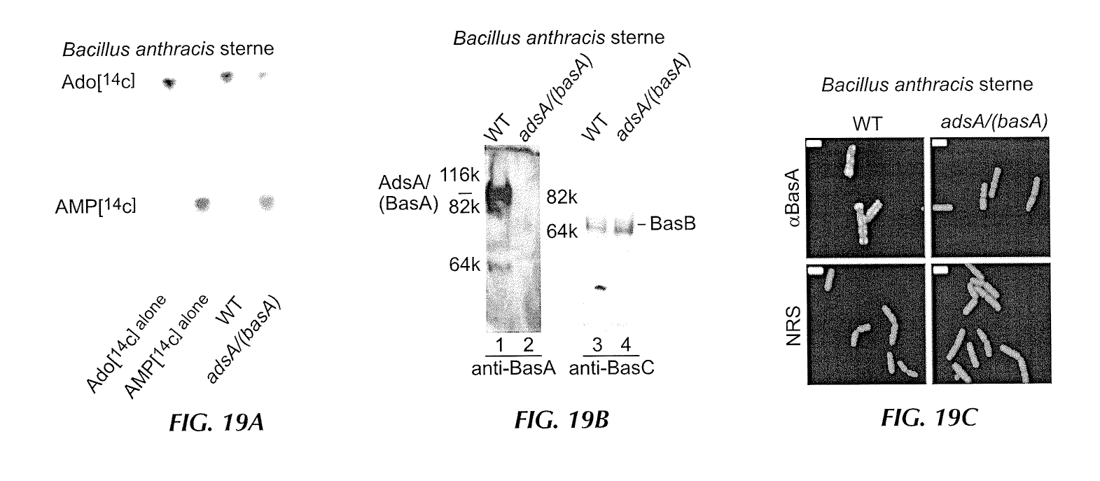

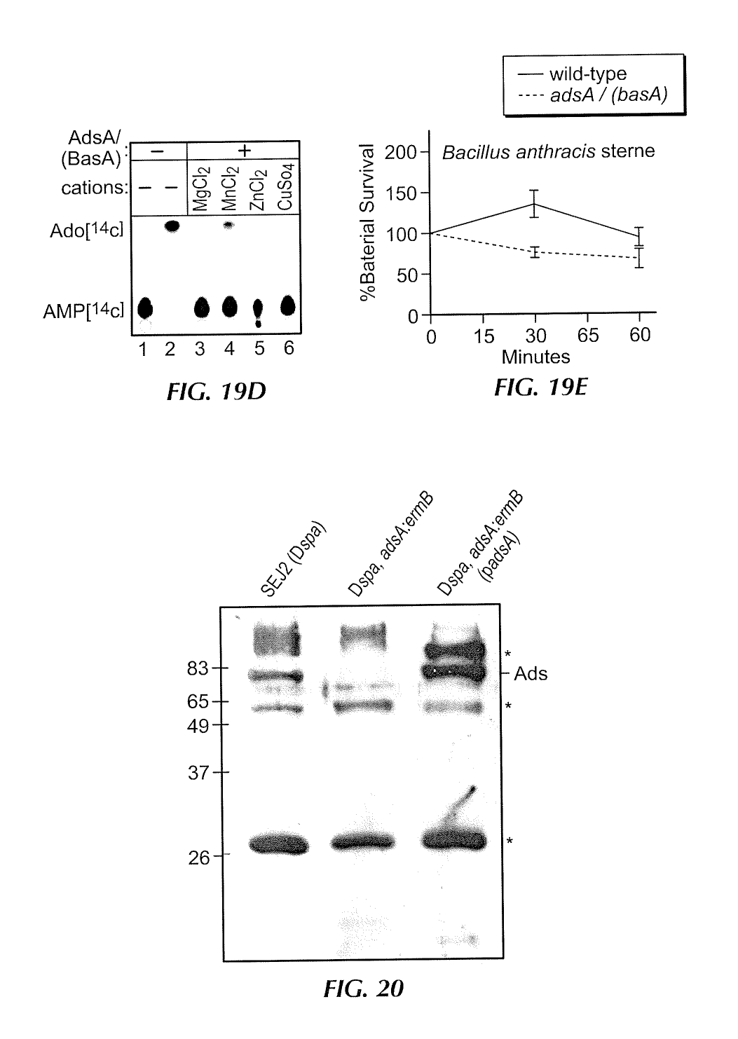

Bacillus anthracis survives in blood and synthesizes adenosine. To investigate whether other pathogenic bacteria also employ adenosine synthase to promote escape from phagocytic clearance, bacterial genome sequences were searched for products harboring the adenosine synthase domain of AdsA and several different genes were identified (Table 3). The genome of B. anthracis encodes BasA (Bacillus anthracis surface protein, NCBI locus tag BAS4031) with a 5′-nucleotidase signature sequence (YdvisLGNHEFN, residues 131-142) and a C-terminal LPXTG sorting signal, indicating that this surface protein is also deposited by sortase A in the cell wall envelope (Gaspar et al. 2005). To determine whether BasA functions as an adenosine synthase and contributes to escape from innate immune responses, we constructed a deletion mutant of basA by allelic replacement (FIG. 19). Mutanolysin, a muralytic enzyme that cleaves N-acetylmuramyl-(β1→4)-N-acetylglucosamine within peptidoglycan (Yokigawa et al., 1974), was used to generate cell wall lysates. Cell wall extracts from wild-type bacilli harbored adenosine synthase activity, however extracts derived from basA mutant bacilli displayed a reduction in this activity (FIG. 19A). Deletion of the structural gene basA abolished expression (FIG. 19B) and surface display of BasA in B. anthracis (FIG. 19C) and reduced the ability of bacilli to synthesize adenosine (FIG. 19A). Residual amounts of AMP hydrolysis may be attributable to other phosphatases, for example alkaline phosphatase. The inventors expressed and affinity purified tagged BasA from E. coli. Similar to S. aureus AdsA, optimal adenosine synthase activity of BasA was observed in the presence of 5 mM MnCl₂ (K_{M}=2.01 nM), whereas 5 mM MgCl₂ showed reduced activity (FIG. 19D). When inoculated into mouse blood, increased phagocytic clearance of the basA mutant was observed, as compared to the wild-type parent B. anthracis Sterne (FIG. 19E). Together these experiments suggest that, similar to staphylococci, B. anthracis also employs AdsA to synthesize adenosine and escape innate immune responses.

TABLE 7
Other microbes with putative 5′-nucleotidases

| | | Pubmed |
|---|---|---|
| Organism | Function | Accession |
| Parasites | | |
| Trichinella spiralis | Secreted 5′-nucleotidase | Q8MQS9 |
| Giardia lamblia | Putative uncharacterized protein | A8BZM2 |
| Gram Positive bacteria | | |
| Bacillus anthracis | 2′,3′-cyclic-nucleotide 2′-phospodiesterase | Q6HTQ7 |
| Bacillus cereus | 5′-nucleotidase domain protein | A7GMX9 |
| Clostridium perfringens | 5′-nucleotidase family protein | B1BIR2 |
| Enterococcus faecalis | 5′-nucleotidase family protein | Q839U0 |
| Listeria monocytogenes | Putative uncharacterized protein | A3FTX4L |
| Listeria monocytogenes | Putative uncharacterized protein | A4DAM1 |
| Staphylococcus aureus str. MW2 | Putative 5′-nucleotidase | Q8NYQ6 |
| Staphylococcus epidermis | 5′-nucleotidase family protein | Q5HQE0 |
| Streptococcus pyogenes | Putative surface-anchored 5′-nucleotidase | A2RF30 |
| Streptococcus mutans | Putative 5′-nucleotidase | Q8CVC5 |
| Streptococcus gordonii | 5′-nucleotidase family protein | A8AXM1 |
| Streptococcus suis | Putative 5′-nucleotidase | A4VV27 |
| Gram Negative bacteria | | |
| Aeromonas salmonicida | Putative 5′-nucleotidase | A4SNE6 |
| Burkholderia dolosa | 5′-nucleotidase/2′,3′-cyclic phosphodiesterase | A2W738 |
| Bacteroides fragilis | Possible secreted 5′-nucleotidase | A5ZBW1 |
| Bacteroides caccai | Putative uncharacterized protein | Q5LHW0 |
| Enterobacter | 5′-nucleotidase domain protein | A4W7G3 |
| Escherichia coli str. UTI89 | Putative uncharacterized protein | Q1R3X2 |
| Haemophilus parasuis | Putative uncharacterized protein | B0QT39 |
| Haemophilus influenzae | Probable 5′-nucleotidase precursor | P44569 |
| Klebsiella pneumoniae | Putative 5′-nucleotidase precursor | A6TGD1 |
| Salmonella choleraesuis | UDP-sugar hydrolase 5′-nucleotidase | Q57S69 |
| Salmonella typhimurium | Putative 5′-nucleotidase | Q7CR96 |
| Salmonella paratyphi A | Putative secreted 5′-nucleotidase | Q5PDK6 |
| Trepenoma denticola | Phosphatase/5′-nucleotidase | Q73PC9 |
| Vibrio cholerae | 5′-nucleotidase precursor | Q9KQ30 |
| Vibrio parahaemolyticus | 5′-nucleotidase precursor | P22848 |
| Yersenia pestis str Antiqua | 5′-nucleotidase precursor | Q1C4S3 |

Other microbes with putative 5′-nucleotidases represent well known pathogens (Table 7) and we sought to analyze their ability to synthesize adenosine. Similar to cell wall extracts from S. aureus and B. anthracis, Enterococcus faecilis and Staphylococcus epidermidis both synthesized adenosine from AMP, whereas the non-pathogenic microbe Bacillus subtilis did not (data not shown). The inventors conclude that the ability of bacterial pathogens to synthesize adenosine and release this immunosuppressive compound into host tissues may represent a universal virulence strategy.

### B. Materials and Methods

Bacterial Strains. S. aureus strains were grown in TSB at 37° C. S. aureus strain USA300 was obtained through the Network on Antimicrobial Resistance in S. aureus (NARSA, NIAID). All mutants used in this study were obtained from the Phoenix (SNE) library (Bae et al., 2004). Each Phoenix isolate is a derivative of the clinical isolate Newman (Duthie and Lorenz, 1952) or USA300 (Carleton et al., 2004) as indicated. All bursa aurealis insertions were transduced into wild-type S. aureus Newman or USA300 using bacteriophage φ85 and verified by PCR analysis. Chloramphenicol was used at 10 mg l⁻¹ for plasmid and allele selection with padsA. Erythromycin was used at 10 mg l⁻¹ for allele selection in S. aureus Newman and at 50 mg l⁻¹ for allele selection in USA300. Mutants of B. anthracis strain Sterne were generated with pLM4, containing a thermosensitive origin of replication. Plasmids with 1 kb DNA sequence flanking each side of the mutation were transformed into B. anthracis and transformants grown at 30° C. (permissive temperature) in LB broth (20 μg ml⁻¹ kanamycin). Cultures were diluted 1:100 and plated on LB agar (20 μg ml⁻¹ kanamycin) at 43° C. overnight (restrictive temperature). Single colonies were inoculated into LB broth without antibiotics and grown overnight at 30° C. To ensure loss of pLM4-based plasmid, these cultures were diluted four times into fresh LB broth without antibiotic pressure and propagated at 30° C. Cultures were diluted and plated on LB agar and colonies examined for kanamycin resistance. DNA from kanamycin-sensitive colonies was analyzed by PCR for the presence or absence of mutant alleles.

Plasmids. The following primers were employed for PCR amplification reactions P55 (5′-TTTCCCGGGACGATCCAGCTCTAATCGCTG-3′) (SEQ ID NO:42), P56 (5′-TTTGAGCTCAAAGCAAATAGATAATCGAGAAATATAAAAAG-3) (SEQ ID NO:43), P57 (5′-TTTGAGCTCAGTTGCTCCAGCCAGCAT T-3′) (SEQ ID NO:44), P58 (5′-TTTGAATTCAAACGGATTCATTCCAGCC-3′) (SEQ ID NO:45), FP10 (5′-TACGAATTCGACTTGGCAGGCAATTGAAAA-3′) (SEQ ID NO:46), RP10 (5′-TGTGAATTCTTAGCTAGCTTTTCTACGTCG-3′) (SEQ ID NO:47), FP3C (5′-TCGGGATCCGCTGAGCAGCATACACCAATG-3′) (SEQ ID NO:48), RPB (5′-TGTGGATCCTTATTGATTAATTTGTTCAGCTAATGC-3′) (SEQ ID NO:49). Ligation of FP10/RP10 (adsA+700 bp upstream from start site) PCR products into pOS1 (EcoRI) generated padsA. Insertion of P55/P56 (basA 1 kb 5′ flanking sequence) and P57/P58 (basA 1 kb 3′ flanking sequence) PCR products into pLM4 (EcoRI, SacI, and XmaI sites) generated pJK34. This plasmid was used to delete the basA coding sequence. Ligation products were transformed into E. coli DH5α, and plasmid DNA into E. coli K1077 (dam⁻, dcm⁻) and purified (non-methylated) plasmid DNA was transformed into B. anthracis following a previously developed protocol (Gaspar et al., 2005). Ligation of FP3C/RPB (1.2 kb truncation of adsA starting 5′ after the signal peptide) PCR products into pGEX-2T (GE Healthcare) generated the adsA expression vector pVT1 and this plasmid was transformed into E. coli BL21.

Animal experiments. All experimental protocols were reviewed, approved and performed under regulatory supervision of The University of Chicago's Institutional Biosafety Committee (IBC) and Institutional Animal Care and Use Committee (IACUC). BALB/c mice were purchased from Charles River Laboratories and Sprague-Dawley rats were purchased from Harlan. Overnight cultures of S. aureus strains were diluted 1:100 into fresh TSB and grown for 3 h at 37° C. Staphylococci were centrifuged, washed twice and diluted in PBS to yield an OD₆₀₀ of 0.5 (1×10⁸ CFU ml⁻¹). Viable staphylococci were enumerated by colony formation on tryptic soy agar plates to quantify the infectious dose. Mice were anaesthetized by intraperitoneal injection of 80-120 mg of ketamine and 3-6 mg of xylazine per kilogram of body weight. One hundred microliters of bacterial suspension (1×10⁷ CFU) were administered intravenously via retro-orbital injection into BALB/c mice (6-wk old female). On day 5, mice were killed by compressed CO₂ inhalation. Kidneys were removed and homogenized in PBS containing 1% Triton X-100. Aliquots of homogenates were diluted and plated on agar medium for triplicate determination of CFU. Student's t-test was performed for statistical analysis using Prizm software. For histopathology, kidney tissue was incubated at room temperature in 10% formalin for 24 h. Tissues were embedded in paraffin, thin-sectioned, stained with haematoxylin-eosin and examined by microscopy.

To measure staphylococcal survival in blood, 6-week old female BALB/c mice were infected with 1×10⁷ CFU of staphylococci by retro-orbital injection. At 30 or 90 minutes, mice were killed by compressed CO₂ inhalation and blood was collected by cardiac puncture using a 25 gauge needle. Aliquots were incubated on ice for 30 minutes in a final concentration of 0.5% saponin/PBS to lyse host eukaryotic cells. Dilutions were plated on TSA for enumeration of surviving CFU at the two different time points.

Chemicals. Mutanolysin (Sigma) was suspended at a concentration of 5,000 units ml⁻¹ in 100 mM sodium phosphate, pH 6.0, containing 1 mM PMSF and stored at −20 C. [¹⁴C]AMP and [¹⁴C]adenosine were purchased from Moravek Biochemicals. Lysostaphin was purchased from AMBI and purified adenosine was purchased from Sigma.

Bacterial survival in blood. Overnight cultures of S. aureus strains were diluted 1:100 into fresh TSB and grown for 3 h at 37° C. Staphylococci were centrifuged, washed twice and diluted in PBS to yield an OD₆₀₀ of 0.5 (1×10⁸ CFU ml⁻¹). Whole blood was collected by cardiac puncture of Sprague-Dawley rats or BALB/c mice and 5 μg ml⁻¹ of lepirudin anticoagulant immediately added. 100 μl of 10⁵ CFU ml⁻¹ of bacteria were mixed with 900 μl of rat or mouse blood. For human blood studies, 100 μl of 10⁸ CFU ml⁻¹ of bacteria was mixed with 900 μl of freshly drawn human blood. The tubes were then incubated at 37° C. with slow rotation for the indicated time points, at which time aliquots were incubated on ice for 30 minutes in a final concentration of 0.5% saponin/PBS to lyse eukaryotic cells. Dilutions of staphylococci were plated on TSA for enumeration of surviving CFU. Experiments with blood from human volunteers involved protocols that were reviewed, approved and performed under regulatory supervision of The University of Chicago's Institutional Review Board (IRB).

Adenosine synthase activity. Overnight cultures of S. aureus strains were diluted 1:100 into fresh TSB and grown for 3 h at 37° C. Staphylococci were centrifuged and washed twice with PBS. 3 ml of cells were spun down and resuspended in 100 μL TSM buffer (50 mM Tris-HCL pH 7.5, 10 mM MgCl₂, and 0.5 M sucrose); 2 μl of lysostaphin was then added and allowed to incubate for 30 min at 37° C. The solution was then spun down for 5 min at 10 k rpm and supernatants containing released cell surface proteins collected. 15 μl of lysostaphin extracts were then incubated with 3 μCi [¹⁴C]AMP for 30 minutes at 37° C. Samples were then spotted on a silica plate followed by separation by TLC using a (75:25 isopropanol: ddH2O) 0.2 M ammonia bicarbonate solvent. For cell wall extracts of S. aureus, E. faecilis, B. anthracis and S. epidermidis digested with mutanolysin, mutanolysin was substituted for lysostaphin and used per the manufacturer's recommended conditions. When assayed with purified proteins, 2 μM of purified AdsA or BasA was incubated in a final volume of 15 μl with 3 μCi [¹⁴C]AMP in the presence of the indicated metal cations in TSM buffer.

Adenosine concentration in blood. Whole blood killing assay with staphylococci was performed as described above. Extraction of plasma was performed as described (Mo and Ballard, 2001). Briefly, after conclusion of the whole blood killing assay, blood samples were centrifuged at 13 k rpm for 5 minutes and non-cellular plasma was collected. 600 μl of plasma was then extracted with 75 μl perchloric acid (1.5 M) and 1 mM EDTA. The supernatant (500 μl) was withdrawn after centrifugation for 5 min at 13 k rpm and neutralized with 29 μl 4 M KOH. After ice cooling for 10 min, the sample was again centrifuged at 13 k rpm for 5 min. The pH of the supernatant was finally adjusted to 6-7, diluted 1:4 with PBS, filtered with a 0.22 μm syringe filter prior to reverse phase high performance liquid chromatography (rpHPLC).

HPLC and mass spectrometry. Presence of adenosine production was determined by rpHPLC. Samples were chromatographed on a 250 mm×3 mm column (BDS Hypersil C18, 5 μm particle size, Thermoscientific). The mobile phase consisted of solution A (dH₂0: 0.1% trifluoroacetic acid) and solution B (acetonitrile: 0.1% trifluoroacetic acid). Adenosine was eluted with a solvent B gradient from 1 to 100%, run from 5 to 50 min. The solvent flow rate was 0.5 ml/min. Peaks were detected by their UV absorbance at 280 nm. The peak of adenosine in the HPLC chromatogram was identified by comparison of its retention time to the retention time of purified adenosine (Sigma) used as a standard sample. Fractions containing adenosine were then co-spotted with matrix (α-cyano-4-hydroxycinnamic acid) and subjected to MALDI-MS under reflector positive conditions.

### REFERENCES

The following references, to the extent that they provide exemplary procedural or other details supplementary to those set forth herein, are specifically incorporated herein by reference.
U.S. Pat. No. 3,791,932
U.S. Pat. No. 3,949,064
U.S. Pat. No. 4,174,384
U.S. Pat. No. 4,197,290
U.S. Pat. No. 4,338,298
U.S. Pat. No. 4,356,170
U.S. Pat. No. 4,367,110
U.S. Pat. No. 4,372,945
U.S. Pat. No. 4,452,901
U.S. Pat. No. 4,474,757
U.S. Pat. No. 4,554,101
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,683,195
U.S. Pat. No. 4,683,202
U.S. Pat. No. 4,690,915
U.S. Pat. No. 4,748,018
U.S. Pat. No. 4,800,159
U.S. Pat. No. 4,879,236
U.S. Pat. No. 5,084,269
U.S. Pat. No. 5,199,942
U.S. Pat. No. 5,221,605
U.S. Pat. No. 5,238,808
U.S. Pat. No. 5,310,687
U.S. Pat. No. 5,322,783
U.S. Pat. No. 5,384,253
U.S. Pat. No. 5,512,282
U.S. Pat. No. 5,538,877
U.S. Pat. No. 5,538,880
U.S. Pat. No. 5,548,066
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,620,896
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,801,234
U.S. Pat. No. 5,840,846
U.S. Pat. No. 5,843,650
U.S. Pat. No. 5,846,709
U.S. Pat. No. 5,846,783
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,871,986
U.S. Pat. No. 5,916,776
U.S. Pat. No. 5,922,574
U.S. Pat. No. 5,925,565
U.S. Pat. No. 5,928,905
U.S. Pat. No. 5,928,906
U.S. Pat. No. 5,932,451
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,958,895
U.S. Pat. No. 5,981,274
U.S. Pat. No. 5,994,624
U.S. Pat. No. 6,008,341
U.S. Pat. No. 6,288,214
U.S. Pat. No. 6,294,177
U.S. Pat. No. 6,482,240
U.S. Pat. No. 6,651,655
U.S. Pat. No. 6,656,462
U.S. Pat. No. 6,733,754
U.S. Pat. No. 6,756,361
U.S. Pat. No. 6,770,278
U.S. Pat. No. 6,793,923
U.S. Pat. No. 6,814,971
U.S. Pat. No. 6,936,258
U.S. Patent Appln. 20030153022
U.S. Patent Appln. 20020169288
Albus et al., Infect. Immun., 59:1008-1014, 1991.
An, J. Virol., 71(3):2292-302, 1997.
Anavi, Sc. thesis from the department of Molecular Microbiology and Biotechnology of the Tel-Aviv University, Israel, 1998.
Andersen et al., J. Immunol., 154, 3359-3372, 1995.
Angel et al., Cell, 49:729, 1987b.
Angel et al., Mol. Cell. Biol., 7:2256, 1987a.
Archer, Clin. Infect. Dis., 26, 1179-1181, 1998.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Baba et al., J. Bacteriol., 190:300-310, 2008.
Baba et al., Lancet., 359:1819-1827, 2007.
Bae and Schneewind, Plasmid, 55:58-63, 2006.
Bae et al., Proc. Natl. Acad. Sci. USA, 101, 12312-12317, 2004.
Banerji et al., Cell, 27(2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Bellus, J. Macromol. Sci. Pure Appl. Chem., A31(1): 1355-1376, 1994.
Berkhout et al., Cell, 59:273-282, 1989.
Blanar et al., EMBO J., 8:1139, 1989.
Bodine and Ley, EMBO J., 6:2997, 1987.
Borges et al., Vet. Parasitol, 143(2):106-11, 2007.
Borrebaeck, In: Antibody Engineering—A Practical Guide, W. H. Freeman and Co., 1992.
Boshart et al., Cell, 41:521, 1985.
Bosze et al., EMBO J., 5(7):1615-1623, 1986.
Braddock et al., Cell, 58:269, 1989.
Brady et al., FEMS Immunol. Med. Microbiol., 52:13-22, 2008.
Brady et al., Infect. Immun., 74:3415-3426, 2006.
Bubeck-Wardenburg et al., Proc. Natl. Acad. Sci. USA, 103:13831-13836, 2006.
Buckley et al. J. Pediatr., 130:378-387, 1997.
Bulla and Siddiqui, J. Virol., 62:1437, 1986.
Burke et al. J. Inf. Dis., 170:1110-1119, 1994.
Burlak et al., Cell Microbiol., 9:1172-1190, 2007.
Burts et al., Proc. Natl. Acad. Sci. USA, 102:1169-1174, 2005.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Carleton et al., Infect. Dis., 190:1730-1738, 2004.
Cassat et al., Methods Mol. Biol., 391:127-144, 2007.
Celander and Haseltine, J. Virology, 61:269, 1987.
Celander et al., J. Virology, 62:1314, 1988.
Champion et al., Science, 313:1632-1636, 2006.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol., 7(8):2745-2752, 1987.
Chen et al. Nat. Chem. Biol., 2:591-5, 2006.
Cheung et al., Proc. Natl. Acad. Sci. USA 89, 6462-6466, 1992.
Choi et al., Cell, 53:519, 1988.
Cocea, Biotechniques, 23(5):814-816, 1997.
Cohen et al., J. Cell. Physiol., 5:75, 1987.
Costa et al., Mol. Cell. Biol., 8:81, 1988.
Cramton et al., Infect. Immun., 67:5427-5433, 1999.
Cripe et al., EMBO J., 6:3745, 1987.
Cronstein et al., Clin. Invest., 85:1150-1157, 1990.
Culotta and Hamer, Mol. Cell. Biol., 9:1376, 1989.
Dalbey and Wickner, J. Biol. Chem., 260:15925-15931, 1985.
Dandolo et al., J. Virology, 47:55-64, 1983.
de Haas, C. J. et al. J. Exp. Med. 199, 687-695 (2004).
De Villiers et al., Nature, 312(5991):242-246, 1984.
Deschamps et al., Science, 230:1174-1177, 1985.
Deussen et al., J. Am. J. Physiol., 264:692-700, 1993.
Devereux et al., Nucl. Acid Res., 12:387-395, 1984.
Diep et al., J. Infect. Dis., 193:1495-1503, 2006a.
Diep et al., Lancet., 367:731-739, 2006b.
Dinges et al., Clin. Microbiol. Rev., 13:16-34, 2000.
Drenkard and Ausubel, Nature, 416, 740-743, 2002.
Duthie and Lorenz, J. Gen. Microbiol., 6:95-107, 1952.
Edbrooke et al., Mol. Cell. Biol., 9:1908, 1989.
Edlund et al., Science, 230:912-916, 1985.
Eltzschig et al. J. Exp. Med., 198:783-796, 2003.
Emorl and Gaynes, Clin. Microbiol. Rev., 6:428-442, 1993.
EP 0 594 610 B1
EP497524
EP497525
Epitope Mapping Protocols In: Methods in Molecular Biology, Vol. 66, Morris (Ed.), 1996.
Fechheimer, et al., Proc Natl. Acad. Sci. USA, 84:8463-8467, 1987.
Feng and Holland, Nature, 334:6178, 1988.
Firak and Subramanian, Mol. Cell. Biol., 6:3667, 1986.
Firestein, G. S. et all. Immunol. 154, 326-334 (1995).
Foecking and Hofstetter, Gene, 45(1):101-105, 1986.
Fortune et al., Proc Natl. Acad. Sci. USA, 102:10676-10681, 2005.
Foster, Nat. Rev. Microbiol., 3:948-958, 2005.
Fournier et al., Infect. Immun. 45:87-93, 1984.
Fraley et al., Proc. Natl. Acad. Sci. USA, 76:3348-3352, 1979.
Fujita et al., Cell, 49:357, 1987.
Gaspar et al., J. Bacteriol., 187, 4646-55, 2005.
GB Appln. 2 202 328
Giblett et al., Lancet., 2:1067-1069, 1972.
Gilles et al., Cell, 33:717, 1983.
Gloss et al., EMBO J., 6:3735, 1987.
Godbout et al., Mol. Cell. Biol., 8:1169, 1988.
Gomez et al., Nature Med., 10:842-848, 2004.
Goodbourn and Maniatis, Proc. Natl. Acad. Sci. USA, 85:1447, 1988.
Goodbourn et al., Cell, 45:601, 1986.
Gopal, Mol. Cell. Biol., 5:1188-1190, 1985.
Gotz, Mol. Microbiol., 43:1367-1378, 2002.
Graham and Van Der Eb, Virology, 52:456-467, 1973.
Greene et al., Immunology Today, 10:272, 1989
Grosschedl and Baltimore, Cell, 41:885, 1985.
Guinn et al., Mol. Microbiol., 51:359-370, 2004.
Hall-Stoodley et al., Nat. Rev. Microbiol., 2:95-108, 2004.
Harland and Weintraub, J. Cell Biol., 101(3):1094-1099, 1985.
Harlow and Lane, Antibodies: A Laboratory Manual, 1988.
Harraghy et al. Microbiology, 151:1789-1800. 2005.
Hartleib et al., Blood, 96:2149-2156, 2000.
Haskó and Pacher, Leukoc. Biol., 83:447-455, 2008.
Haslinger and Karin, Proc. Natl. Acad. Sci. USA, 82:8572, 1985.
Hauber and Cullen, J. Virology, 62:673, 1988.
Heilmann et al., Mol. Microbiol., 24:1013-1024, 1997.
Hen et al., Nature, 321:249, 1986.
Hensel et al., Lymphokine Res., 8:347, 1989.
Herr and Clarke, Cell, 45:461, 1986.
Higgins et al., FEMS Microbiol. Lett., 258:290-296, 2006.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Hsu et al., Proc. Natl. Acad. Sci. USA, 100:12420-12425, 2003.
Huang et al., Cell, 27:245, 1981.
Hug et al., Mol. Cell. Biol., 8:3065, 1988.
Hussain et al., J. Bacteriology, 183:6778-86. 2001.
Huston et al., In: Methods in Enzymology, Langone (Ed.), Academic Press, NY, 203:46-88, 1991.
Hwang et al., Mol. Cell. Biol., 10:585, 1990.
Ichiman and Yoshida, J. Appl. Bacterid., 51:229-241, 1981.
Ichiman et al., J. Appl. Bacterid., 71:176-181, 1991.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., Proc Natl Acad Sci USA, 85(24):9436-9440, 1988.
Inouye and Inouye, Nucleic Acids Res., 13: 3101-3109, 1985.
Iwamoto et al., Planta Med., 54(5):422-5, 1988.
Jakobovits et al., Mol. Cell. Biol., 8:2555, 1988.
Jameel and Siddiqui, Mol. Cell. Biol., 6:710, 1986.
Jardetzky et al., Nature, 368:711-718, 1994.
Jaynes et al., Mol. Cell. Biol., 8:62, 1988.
Jensen, Acta Path. Microbiol. Scandin., 44:421-428, 1958.
Jin et al., J. Immunol., 172:1169-1176, 2004.
Johnson et al., Methods in Enzymol., 203:88-99, 1991.
Johnson et al., Mol. Cell. Biol., 9:3393, 1989.
Johnson et al., Infect. Immun. 76, 1756-1765, 2008.
Johnstone et al., In: Immunochemistry in Practice, Blackwell Scientific Publications, Oxford, 1982.
Jones, Carb. Research, 340:1097-1106, 2005.
Jonsson et al., J. Infect. Dis., 185:1417-1424, 2002.
Joyce et al., Carbohydrate Res., 338:903-922, 2003.
Kadesch and Berg, Mol. Cell. Biol., 6:2593, 1986.
Kaeppler et al., Plant Cell Rep., 8:415-418, 1990.
Kaneda et al., Science, 243:375-378, 1989.
Karin et al., Mol. Cell. Biol., 7:606, 1987.
Katinka et al., Cell, 20:393, 1980.
Kato et al., J. Biol. Chem., 266:3361-3364, 1991.
Kawamoto et al., Mol. Cell. Biol., 8:267, 1988.
Khoa et al. J. Immunol., 167:4026-4032, 2001.
Kiledjian et al., Mol. Cell. Biol., 8:145, 1988.
Klamut et al., Mol. Cell. Biol., 10:193, 1990.
Klevens et al., Clin. Infect. Dis., 42, 389-91, 2006.
Klevens et al., Jama, 298:1763-71, 2007.
Koch et al., Mol. Cell. Biol., 9:303, 1989.
Kohler and Milstein, Nature 256:495-497, 1975.
Kolter and Greenberg, Nature 441, 300-302, 2006.
Kriegler and Botchan, In: Eukaryotic Viral Vectors, Gluzman (Ed.), Cold Spring
Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
Kuroda et al., Lancet., 357:1225-1240, 2001.
Kyte and Doolittle, J. Mol. Biol., 157(1):105-132, 1982.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Nature, 294:228, 1981.
Lee et al., Nucleic Acids Res., 12:4191-206, 1984.
LeeTrends Microbiol., 4(4):162-166, 1996.
Levenson et al., Hum. Gene Ther., 9(8):1233-1236, 1998.
Levinson et al., Nature, 295:79, 1982.
Lin et al., Mol. Cell. Biol., 10:850, 1990.
Lowy, New Engl. J. Med., 339:520-532, 1998.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
MacGurn et al., Mol. Microbiol., 57:1653-1663, 2005.
Macket al., J. Bacteriol. 178, 175-183, 1996.
Maira-Litran et al., Infect. Immun., 70:4433-4440, 2002.
Maira-Litran et al., Vaccine, 22:872-879, 2004.
Majors and Varmus, Proc. Natl. Acad. Sci. USA, 80:5866, 1983.
Mazmanian et al., Mol. Microbiol. 40, 1049-1057, 2001.
Mazmanian et al., Proc. Natl. Acad. Sci. USA, 99:2293-2298, 2002.
Mazmanian et al., Science, 285, 760-763, 1999.
Mazmanian et al., Science, 299:906-909, 2003.
McColl et al. FASEB J., 20:187-189, 2006.
McDevitt et al., Mol. Microbiol., 11:237-248, 1994.
McDougal et al., J. Clin. Microbiol., 41:5113-5120, 2003.
McLaughlin et al., PLoS Pathog., 3:e105, 2007.
McNeall et al., Gene, 76:81, 1989.
Merck Manual Of Diagnosis And Therapy, §13, Ch. 157, 100^{th} Ed. (Beers & Berkow, Eds., 2004
Mernaugh et al., In: Molecular Methods in Plant Pathology, Singh et al. (Eds.), CRC Press Inc., Boca Raton, Fla., 359-365, 1995.
Merrifield, Science, 232(4748):341-347, 1986.
Miksicek et al., Cell, 46:203, 1986.
Mo and Ballard, J. Physiol., 536:593-603, 2001.
Mordacq and Linzer, Genes and Dev., 3:760, 1989.
Moreau et al., Carbohydrate Res., 201:285-297, 1990.
Moreau et al., Nucl. Acids Res., 9:6047, 1981.
Mosmann and Coffman, Ann. Rev. Immunol., 7:145-173, 1989.
Muesing et al., Cell, 48:691, 1987.
Musher et al., Medicine (Baltimore), 73:186-208, 1994.
Needleman & Wunsch, J. Mol. Biol., 48:443, 1970.
Nemeth et al. J. Immunol., 176:5616-5626, 2006.
Ng et al., Nuc. Acids Res., 17:601, 1989.
Ni Eidhin et al., Mol. Microbiol., 30:245-257, 1998.
Nicolau and Sene, Biochim. Biophys. Acta, 721:185-190, 1982.
Nicolau et al., Methods Enzymol., 149:157-176, 1987.
Novick, Mol. Microbiol., 48:1429-1449, 2003.
Novick and Jiang. Microbiology 149, 2709-17 (2003).
Omirulleh et al., Plant Mol. Biol., 21(3):415-28, 1993.
Ondek et al., EMBO J., 6:1017, 1987.
O'Riordan and Lee, Clin. Microbiol. Rev., 17:218-234, 2004.
Ornitz et al., Mol. Cell. Biol., 7:3466, 1987.
Pallen, Trends Microbiol., 10:209-212, 2002.
Palmiter et al., Nature, 300:611, 1982.
Palmqvist et al., Microbes Infect., 6:188-195, 2004.
Panther et al., FASEB J., 15:1963-1970, 2001.
PCT Appln. PCT/US89/01025
PCT Appln. WO 00/02523
PCT Appln. WO 00/12689
PCT Appln. WO 01/60852
PCT Appln. WO 03/53462
PCT Appln. WO 04/43405
PCT Appln. WO 04/43407
PCT Appln. WO 95/08348
Pearson & Lipman, Proc. Natl. Acad. Sci. USA, 85:2444, 1988.
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Peschel and Sahl, Nat. Rev. Microbiol., 4:529-536, 2006.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potrykus et al., Mol. Gen. Genet., 199(2):169-177, 1985.
Pugsley, Microbiol. Rev., 57:50-108, 1993.
Pym et al., Mol. Microbiol., 46; 709-717, 2002.
Pym et al., Nat. Med., 9:533-539, 2003.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Remington's Pharmaceutical Sciences, 18th Ed. Mack Printing Company, 1289-1329, 1990.
Resendez Jr. et al., Mol. Cell. Biol., 8:4579, 1988.
Ripe et al., Mol. Cell. Biol., 9:2224, 1989.
Rippe, et al., Mol. Cell. Biol., 10:689-695, 1990.
Rittling et al., Nuc. Acids Res., 17:1619, 1989.
Roben et al., J. Immunol., 154:6437-6445, 1995.
Rooijakkers et al., Trends Microbiol., 13:596-601, 2005.
Rosen et al., Cell, 41:813, 1988.
Sakai et al., Genes and Dev., 2:1144, 1988.
Sambrook et al., In: Molecular cloning, Cold Spring Harbor Laboratory Press, Cold Spring Harbor, N.Y., 2001.
Sau et al., Microbiology, 143:2395-2405, 1997.
Schaffner et al., J. Mol. Biol., 201:81, 1988.
Schindler and Schuhardt, Proc. Natl. Acad. Sci. USA, 51:414-421, 1964.
Scriba, T. J. et al. The Infect. Immun. 76, 2164-2168 (2008).
Searle et al., Mol. Cell. Biol., 5:1480, 1985.
Sharp and Marciniak, Cell, 59:229, 1989.
Shaul and Ben-Levy, EMBO J., 6:1913, 1987.
Shaw et al., Microbiology, 150:217-228, 2004.
Sherman et al., Mol. Cell. Biol., 9:50, 1989.
Sibbald et al., Microbiol. Mol. Biol. Rev., 70:755-788, 2006.
Singh et al., Nature, 417, 552-555, 2002.
Sleigh and Lockett, J. EMBO, 4:3831, 1985.
Smith & Waterman, Adv. Appl. Math., 2:482, 1981.
Sorensen et al., Infect. Immun., 63:1710-1717, 1995.
Spalholz et al., Cell, 42:183, 1985.
Spandau and Lee, J. Virology, 62:427, 1988.
Spandidos and Wilkie, EMBO J., 2:1193, 1983.
Stanley et al., Proc. Natl. Acad. Sci. USA, 100:13001-13006, 2003.
Stephens and Hentschel, Biochem. J., 248:1, 1987.
Stewart and Young, In: Solid Phase Peptide Synthesis, 2d. ed., Pierce Chemical Co., 1984.
Stranger-Jones et al., Proc. Nat. Acad. Sci. USA, 103:16942-16947, 2006.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiel et al., Microbes Infect., 5:515-526, 2003.
Thiesen et al., J. Virology, 62:614, 1988.
Thompson et al., Biochem. Biophys. Res. Commun., 145:118-125, 1987.
Thompson et al., J. Immunol., 143:1815-1821, 1989.
Thomson et al., J. Immunol., 157(2):822-826, 1996.
Tigges et al., J. Immunol., 156(10):3901-3910, 1996.
Toukairin et al., Chem. Pharm. Bull. (Tokyo), 39(6):1480-3, 1991.
Treisman, Cell, 42:889, 1985.
Tronche et al., Mol. Biol. Med., 7:173, 1990.
Trudel and Constantini, Genes and Dev., 6:954, 1987.
Tyndell et al., Nuc. Acids. Res., 9:6231, 1981.
Uchino et al., Planta. Medt., 54(5):419-22, 1988.
van den Ent and Lowe, FEBS Lett., 579:3837-3841, 2005.
van Wely et al., FEMS Microbiol. Rev., 25:437-454, 2001.
Vannice and Levinson, J. Virology, 62:1305, 1988.
Vasseur et al., Proc Natl. Acad. Sci. USA, 77:1068, 1980.
Vaughan et al., Nat. Biotech., 16:535-539, 1998.
Voyich et al., J. Immunol., 175:3907-3919, 2005.
Vuong et al., J. Biol. Chem., 279:54881-54886, 2004.
Wang and Calame, Cell, 47:241, 1986.
Wang et al., Nat. Med., 13:1510-1514, 2007.
Weber et al., Cell, 36:983, 1984.
Weinberger et al. Mol. Cell. Biol., 8:988, 1984.
Weiss et al., J. Antimicrob. Chemother., 53:480-486, 2004.
Winoto and Baltimore, Cell, 59:649, 1989.
Wong et al., Gene, 10:87-94, 1980.
Xie, C. et al. J. Exp. Med. 203, 985-994 (2006).
Xiong et al. Antimicrob. Agents Chemother., 49:380-387, 2005.
Xu et al., J. Infect. Dis., 189:2323-2333, 2004.
Xu et al., Mol. Microbiol., 66(3):787-800, 2007.
Yang et al. J. Immunol., 175:6458-6464, 2005.
Yokogawa et al., Antimicrob. Agents Chemother., 6:156-165, 1974.
Yutzey et al. Mol. Cell. Biol., 9:1397, 1989.
Zimmermann, Biochem. J., 285:345-365, 1992.

1. An antigen composition comprising:
(a) an isolated Emp antigen, or an immunogenic fragment thereof; and
(b) at least one additional staphylococcal antigen selected from a group consisting of an isolated ClfA, CIfB, Eap, EsaB, EsxA, EsxB, EsaC, IsdA, IsdB, IsdC, SasF, SasB, SdrC, SdrD, SdrE, SasH, Ebh, Coa, vWa, Hla and SpA antigen, or an immunogenic fragment thereof,
wherein the antigens are comprised in a pharmaceutically acceptable composition capable of stimulating an immune response in a subject in need thereof.

2. The composition of claim 1, further comprising one or more of a type V capsular saccharide, a type VIII capsular saccharide, and/or a polysaccharide intracellular adhesin (PIA).

3. (canceled)

4. (canceled)

5. The composition of claim 1, wherein the at least one additional staphylococcal antigen is selected from the group consisting of an isolated ClfB, Eap, EsxA, EsxB, IsdA, and SrdD.

6. The composition of claim 1, wherein the at least one additional staphylococcal antigen is an isolated IsdA antigen.

7. The composition of claim 6, further comprising one or more additional staphylococcal antigens selected from the group consisting of ClfB, Eap, EsxA, EsxB, Hla, and SdrD.

8. The composition of claim 7, further comprising one or more of a type V capsular saccharide, a type VIII capsular saccharide, and/or a polysaccharide intracellular adhesin (PIA).

9. (canceled)

10. (canceled)

11. The composition of claim 1, wherein the composition comprises less than a 1% weight/weight contamination with other staphylococcal bacterial components.

12. The composition of claim 1, wherein one or more isolated antigens are coupled to an adjuvant.

13. The composition of claim 1, wherein the Emp antigen comprises at least 5, 10, 15, or 20 consecutive amino acids of SEQ ID NO:2.

14. The composition of claim 1, wherein the Emp antigen is at least 70%, 80%, 90%, 95%, or 98% identical to SEQ ID NO:2.

15. (canceled)

16. (canceled)

17. The composition of claim 1, wherein the Emp antigen comprises the amino acid sequence of SEQ ID NO:2.

18. (canceled)

19. An antigen comprising two or more of a CIfA, CIfB, Eap, Emp, EsaB, EsxA, EsxB, EsaC, IsdA, IsdB, IsdC, SasF, SasH, Ebh, Coa, vWa, Hla, SasB, SdrC, SdrD, SdrE, and SpA antigen, wherein the two or more antigens are coupled forming a multimeric antigen.

20-30. (canceled)

31. A method for eliciting an immune response against a staphylococcus bacterium in a subject comprising administering to the subject an effective amount of a composition comprising:
(a) an isolated Emp antigen, or an immunogenic fragment thereof, and
(b) at least one additional staphylococcal antigen selected from a group consisting of an isolated ClfA, ClfB, Eap, EsaB, EsxA, EsxB, EsaC, Hla, SasB, SasH, Ebh, Coa, vWa, IsdA, IsdB, IsdC, SasF, SdrC, SdrD, SdrE, and SpA antigen, or an immunogenic fragment thereof.

32. The method of claim 31, wherein the subject is provided with an effective amount of an isolated antigen by administering to the subject a composition comprising:
i) the isolated antigen, or
ii) at least one isolated recombinant nucleic acid molecule encoding the antigen.

33. The method of claim 31, where the subject is also administered an adjuvant.

34. (canceled)

35. (canceled)

36. The method of claim 31, wherein the Emp antigen comprises at least 5, 10, 15, or 20 consecutive amino acids of SEQ ID NO:2.

37. The method of claim 31, wherein the Emp antigen is at least 70%, 80%, 90%, 95%, or 98% identical to SEQ ID NO:2.

38. (canceled)

39. (canceled)

40. The method of claim 31, wherein the Emp antigen comprises the amino acid sequence of SEQ ID NO:2.

41. The method of claim 31, wherein the staphylococcus bacterium is a S. aureus bacterium.

42. The method of claim 31, wherein the stapylococcus bacterium is a drug resistant bacterium.

43-52. (canceled)

53. The method of claim 31, wherein the composition includes a recombinant, non-staphylococcus bacterium expressing an isolated Emp antigen, or an immunogenic fragment thereof, and at least one additional staphylococcal antigen selected from a group consisting of an isolated ClfA, CIfB, Eap, EsaB, EsxA, EsxB, EsaC, Hla, SasB, SasH, Ebh, Coa, vWa, IsdA, IsdB, IsdC, SasF, SdrC, SdrD, SdrE, and SpA antigen, or an immunogenic fragment thereof.

54. (canceled)

55. (canceled)

56. The method of claim 31, wherein the subject is human.

57. The method of claim 31, wherein the immune response is a protective immune response.

58-71. (canceled)

72. A method for limiting staphylococcal abscess formation and/or persistence in a subject comprising providing to a subject having or suspected of having or at risk of developing a staphylococcal infection an effective amount of an isolated Emp antigen or an immunogenic fragment thereof, and at least one additional staphylococcal antigen selected from a group consisting of an isolated ClfA, ClfB, Eap, EsaB, EsaC, EsxA, EsxB, IsdA, IsdB, IsdC, SasB, SasH, Ebh, Coa, vWa, SasF, SdrC, SdrD, SdrE, Hla and SpA antigen, or an immunogenic fragment thereof.

73-97. (canceled)

98. A method for eliciting an immune response against a staphylococcus bacterium in a subject comprising administering to the subject an effective amount of a composition comprising:
(a) an isolated Eap antigen, or an immunogenic fragment thereof, and
(b) at least one additional staphylococcal antigen selected from a group consisting of an isolated ClfA, ClfB, Emp, EsaB, EsaC, EsxA, EsxB, IsdA, IsdB, IsdC, SasF, SasH, Ebh, Coa, vWa, SasB, SdrC, SdrD, SdrE, Hla and SpA antigen, or an immunogenic fragment thereof.

99-162. (canceled)

163. A method for eliciting an immune response against a staphylococcus bacterium in a subject comprising administering to the subject an effective amount of a composition comprising an isolated AdsA peptide, or an immunogenic fragment thereof.

164-215. (canceled)


SEQUENCE LISTING (53 sequences)

SEQ ID NO: 1 - DNA, 1023 bp, Staphylococcus aureus
  CDS (1)..(1023)
atgaaaaagaaattattagttttaactatgagcacgctatttgctacacaaattatgaattcaaatcacgctaaagcatcagtgacagagagtgttgacaaaaaatttgtagttccagaatcaggaattaataaaattattccagcttacgatgaatttaagaattcgccaaaagtaaatgttagtaatttaactgacaataaaaactttgtagcttctgaagataaattgaataagattgcagattcatcggcagctagtaaaattgtagataaaaactttgtcgtaccagaatcaaagttaggaaacattgtgccagagtacaaagaaatcaataatcgcgtgaatgtagcaacaaacaatccagcttcacaacaagttgataagcattttgttgctaaaggcccagaagtaaatagatttattacgcaaaacaaagtaaaccaccacttcattactacgcaaacccactacaagaaagttattacttcatacaaatcaacacatgtacataaacatgtaaatcatgcaaaggattctattaataaacactttattgttaaaccatcagaatcgcctagatatacacatccatctcaatctttaattatcaagcatcattttgcagttcctggatatcacgcgcataaatttgttacaccagggcatgctagcattaaaattaatcacttttgtgttgtgccacaaataaatagtttcaaggtaattccaccatatggtcacaattcacatcgtatgcatgtaccaagtttccaaaataacacaacagcaacacatcaaaatgctaaagtaaataaagcatatgactataaatacttctattcttataaagtagttaaaggtgtgaagaaatatttctcattttcacaatcaaatggttataaaattgggaaaccatcattaaatatcaaaaatgtaaattatcaatatgctgttccaagttatagccctacacactacgttcctgaatttaagggtagcttaccagcaccacgagtataa

SEQ ID NO: 2 - protein, 340 aa, Staphylococcus aureus
MKKKLLVLTMSTLFATQIMNSNHAKASVTESVDKKFVVPESGINKIIPAYDEFKNSPKVNVSNLTDNKNFVASEDKLNKIADSSAASKIVDKNFVVPESKLGNIVPEYKEINNRVNVATNNPASQQVDKHFVAKGPEVNRFITQNKVNHHFITTQTHYKKVITSYKSTHVHKHVNHAKDSINKHFIVKPSESPRYTHPSQSLIIKHHFAVPGYHAHKFVTPGHASIKINHFCVVPQINSFKVIPPYGHNSHRMHVPSFQNNTTATHQNAKVNKAYDYKYFYSYKVVKGVKKYFSFSQSNGYKIGKPSLNIKNVNYQYAVPSYSPTHYVPEFKGSLPAPRV

SEQ ID NO: 3 - DNA, 1755 bp, Staphylococcus aureus
  CDS (1)..(1755)
atgaaatttaagtcattgattacaacaactttagcattaggtgttttagcatcaacaggtgcaaactttaataataatgaagcgtctgccgcagctaagccattagataaatcatcaagttcgttacaccatggatattctaaagtccatgttccatatgcaatcactgtgaacggtacaagccaaaatattttatcaagcttaacatttaataagaatcaaaatattagttataaagatttagaggatagagttaaatcagttttaaaatcagacagaggtattagtgatatagatttaagactatcgaagcaagcgaaatatactgtttactttaaaaatggaacaaagaaagttatcgatttgaaagcaggtatttacacagctgatttaattaatacaagtgaaattaaagctattaatattaacgtagatactaaaaagcaagttgaagataaaaagaaagataaagcaaattaccaagttccatacacaatcactgtgaacggtacaagccaaaatattttatcaaacttaacatttaataagaatcaaaatattagttacaaagatttagaggataaagttaaatcagttttagaatcaaatagaggtattaccgatgttgatttaagattatcgaagcaagcgaaatatacagttaattttaaaaatggaacgaagaaagttatcgatttgaaatcaggtatttacacagcgaatttaataaattcaagtgatattaaaagtatcaatattaacgtagatacaaaaaaacatatcgaaaataaagctaaaagaaactatcaagttccatattcaattaatttaaatggtacatctacaaacattttatcgaatctttcattttcaaataaaccttggacaaattacaaaaatttaactagtcaaataaaatcagtactgaagcatgatagaggtattagtgaacaagatttaaaatatgctaagaaagcttattatactgtttattttaaaaatggtggtaaaagaatcttacagttgaattcaaaaaattacacagcaaacttagttcatgcgaaagatgttaagagaattgaaattactgttaaaacaggaactaaagcgaaagcagacagatatgtaccatacacaattgcagtaaatggcacatcaacaccaattttatcagatttaaaatttacaggtgacccacgtgtaggctacaaagatatctctaaaaaagttaaatcagtattgaagcatgatagaggtatcggggaacgtgaattaaaatatgcaaaaaaagcaacttacacagtacattttaaaaatggaacgaaaaaagtgattaacataaattcaaatattagccaactgaatctgctttatgttcaagatattaaaaagatagatattgatgttaaaacaggaactaaagcgaaagcggatagctatgtaccatatacaattgcagtaaatggcacatcaacaccaattttatcaaaacttaaaatttcgaataaacaattaattagttacaaatatttaaatgacaaagtgaaatctgtattaaaaagtgaaagaggcatcagtgatcttgacttaaaatttgcgaaacaagcaaaatatacagtatatttcaaaaatggaaagaaacaagtagtgaatttaaaatcagacatctttacacctaatttatttagtgccaaagatattaaaaagattgatattgatgtaaaacaatacactaaatcaaaaaaaaataaataa

SEQ ID NO: 4 - protein, 584 aa, Staphylococcus aureus
MKFKSLITTTLALGVLASTGANFNNNEASAAAKPLDKSSSSLHHGYSKVHVPYAITVNGTSQNILSSLTFNKNQNISYKDLEDRVKSVLKSDRGISDIDLRLSKQAKYTVYFKNGTKKVIDLKAGIYTADLINTSEIKAININVDTKKQVEDKKKDKANYQVPYTITVNGTSQNILSNLTFNKNQNISYKDLEDKVKSVLESNRGITDVDLRLSKQAKYTVNFKNGTKKVIDLKSGIYTANLINSSDIKSININVDTKKHIENKAKRNYQVPYSINLNGTSTNILSNLSFSNKPWTNYKNLTSQIKSVLKHDRGISEQDLKYAKKAYYTVYFKNGGKRILQLNSKNYTANLVHAKDVKRIEITVKTGTKAKADRYVPYTIAVNGTSTPILSDLKFTGDPRVGYKDISKKVKSVLKHDRGIGERELKYAKKATYTVHFKNGTKKVININSNISQLNLLYVQDIKKIDIDVKTGTKAKADSYVPYTIAVNGTSTPILSKLKISNKQLISYKYLNDKVKSVLKSERGISDLDLKFAKQAKYTVYFKNGKKQVVNLKSDIFTPNLFSAKDIKKIDIDVKQYTKSKKNK

SEQ ID NO: 5 - DNA, 294 bp, Staphylococcus sp.
  CDS (1)..(294)
atggcaatgattaagatgagtccagaggaaatcagagcaaaatcgcaatcttacgggcaaggttcagaccaaatccgtcaaattttatctgatttaacacgtgcacaaggtgaaattgcagcgaactgggaaggtcaagctttcagccgtttcgaagagcaattccaacaacttagtcctaaagtagaaaaatttgcacaattattagaagaaattaaacaacaattgaatagcactgctgatgccgttcaagaacaagaccaacaactttctaataatttcggtttgcaataa

SEQ ID NO: 6 - protein, 97 aa, Staphylococcus sp.
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 7 - DNA, 307 bp, Staphylococcus sp.
  CDS (1)..(306)
atgggtggatataaagggattaaagcagatggtggcaaggtgaatcaagcgaaacaattagcggcaaaaatagctaaagatattgaagcatgtcaaaagcaaacgcaacagctcgctgagtatatcgaaggtagtgattgggaaggacagttcgccaataaggtgaaagatgtgttacttattatggcaaagtttcaagaagaattagtacaaccgatggctgaccatcaaaaagcaattgataacttaagtcaaaatctagcgaaatacgatacattatcaattaagcaaggacttgatagggtga

SEQ ID NO: 8 - protein, 102 aa, Staphylococcus sp.
MGGYKGIKADGGKVNQAKQLAAKIAKDIEACQKQTQQLAEYIEGSDWEGQFANKVKDVLLIMAKFQEELVQPMADHQKAIDNLSQNLAKYDTLSIKQGLDRV

SEQ ID NO: 9 - DNA, 4158 bp, Staphylococcus sp.
  CDS (1)..(4158)
atgctaaacagagaaaataaaacggcaataacaagaaaaggcatggtatccaatcgattaaataaattttcgattagaaagtacacagtgggaacagcatcaattttagtaggtacaacattaatttttggtctggggaaccaagaagcaaaggctgcagaaagtactaataaagaattgaacgaagcgacaacttcagcaagtgataatcaatcgagtgataaagttgatatgcagcaactaaatcaagaagacaatactaaaaatgataatcaaaaagaaatggtatcatctcaaggtaatgaaacgacttcaaatgggaataaatcaatagaaaaagaaagtgtacaatctaccactggaaataaagttgaagtttcaactgccaaatcagatgagcaagcttcaccaaaatctacgaatgaagatttaaacactaaacaaactataagtaatcaagaagggttacaacctgatttgctagagaataaatcagtggtaaatgttcaaccaactaatgaggaaaacaaaaaggtagatgcgaaaactgaatcaactacattaaatgttaaaagtgatgctatcaagagtaatgctgaaactcttgttgataacaatagtaattcaaataatgaaaataatgcagatatcattttgccaaaaagtacagcacctaaaagtttgaatacaagaatgcgtatggcagcaatacaaccaaactcaacagattctaaaaatgttaatgatttaatcacatcaaatacaacattaactgtcgttgatgcagataatagcaaaacgattgtaccagcccaagattatttatcattaaaatcacaaattacagttgatgacaaagttaaatcaggtgattatttcacaattaaatactcagatacagtacaagtatatggattgaatccggaagatattaaaaatattggtgatattaaagatccaaataatggtgaaacaattgcgactgcaaaacatgatactgcaaataatttaattacatatacatttacagattatgttgatcgatttaattcagtaaaaatgggtattaattactcaatttatatggatgcagatacaattcctgttgacaagaaagatgttccttttagtgtaactattggaaatcaaattacaactacaacagcagatatcacttatccggcttataaagaagctgacaataattcaataggatcagcttttacagagacagtttctcatgtaggaaatgttgaagaccctggttactataaccaggtagtatatgttaatcctatggataaggatttaaaaggtgctaagttaaaagttgaagcgtaccatccgaaatatccaactaatattggtcaaattaatcaaaatgttacaaatataaaaatatatcgtgttcctgaaggatatacattgaataaaggatatgacgttaatactaatgatttggtagacgtaactgatgaatttaaaaataaaatgacgtatggatcaaatcaaagtgttaatcttgattttggtgatattacatcagcatatgttgtaatggttaatacaaaattccaatatacaaatagcgaaagcccaacacttgttcaaatggctactttatcttcaacaggtaataaatccgtttctactggcaatgctttaggatttactaataaccaaagtggcggagctggtcaagaagtatataaaattggtaactacgtatgggaagatactaataaaaacggtgttcaagaattaggagaaaaaggcgttggcaatgtaactgtaactgtatttgataataatacaaatacaaaagtaggagaagcagttactaaagaagatgggtcatacttgattccaaacttacctaatggagattaccgtgtagaattttcaaacttaccaaaaggttatgaagtaaccccttcaaaacaaggtaataacgaagaattagattcaaacggcttatcttcagttattacagttaatggcaaagataacttatctgcagacttaggtatttacaaacctaaatacaacttaggtgactatgtctgggaagatacaaataaaaatggtatccaagaccaagatgaaaaaggtatatctggcgtaacggtaacattaaaagatgaaaacggtaacgtgttaaaaacagttacaacagacgcagatggcaaatataaatttactgatttagataatggtaattataaagttgaatttactacaccagaaggctatacaccgactacagtaacatctggtagcgacattgaaaaagactctaatggtttaacaacaacaggtgttattaatggtgctgataacatgacattagatagtggattctacaaaacaccaaaatataatttaggtaattatgtatgggaagatacaaataaagatggtaagcaggattcaactgaaaaaggtatttcaggcgtaacagttacattgaaaaatgaaaacggtgaagttttacaaacaactaaaacagataaagatggtaaatatcaatttactggattagaaaatggaacttataaagttgaattcgaaacaccatcaggttacacaccaacacaagtaggttcaggaactgatgaaggtatagattcaaatggtacatcaacaacaggtgtcattaaagataaagataacgatactattgactctggtttctacaaaccgacttacaacttaggtgactatgtatgggaagatacaaataaaaacggtgttcaagataaagatgaaaagggtatttcaggtgtaacagttacgttaaaagatgaaaacgacaaagttttaaaaacagttacaacagatgaaaatggtaaatatcaattcactgatttaaacaatggaacttataaagttgaattcgagacaccatcaggttatacaccaacttcagtaacttctggaaatgatactgaaaaagattctaatggtttaacaacaacaggtgtcattaaagatgcagataacatgacattagacagtggtttctataaaacaccaaaatatagtttaggtgattatgtttggtacgacagtaataaagacggcaaacaagattcaactgaaaaaggtatcaaagatgttaaagttattttattaaatgaaaaaggcgaagtaattggaacaactaaaacagatgaaaatggtaaataccgctttgataatttagatagcggtaaatacaaagttatttttgaaaagcctactggcttaacacaaacaggtacaaatacaactgaagatgataaagatgccgatggtggcgaagttgatgtaacaattacggatcatgatgatttcacacttgataatggctactacgaagaagaaacatcagatagcgactcagattcggacagcgattcagactcagacagcgattcagactcagatagtgattcagattcagatagtgattcagattcagatagtgattcagattcagacagcgactcagactcagatagtgactcagactcagatagcgattcagattcagatagcgattcagactcagacagcgattcagattcagacagcgactcagactcagatagcgactcagattcggacagcgattcagactcagatagcgactcagactcagacagcgattcagactcagatagcgactcagactcagatagcgattcagattcagacagcgattcagattcagacagtgattcagattcagacagcgactcagattcagatagcgattcagactcagactcagatagcgattcagattcagacagcgactcagattcggacagcgactcagactcagacagtgattcagattcagatagcgactcagactcagatagcgactcagattcagacagcgattcagactcagatagtgactcagattcggacagcgattcagactcagatagcgactcagattcagacagtgattcagactcagatgcaggtaagcacacacctgttaaaccaatgagtactactaaagaccatcacaataaagcaaaagcattaccagaaacaggtaatgaaaatagtggctcaaataacgcaacgttatttggcggattattcgcagcattaggatcattattgttattcggtcgtcgtaaaaaacaaaataaataa

SEQ ID NO: 10 - protein, 1385 aa, Staphylococcus sp.
MLNRENKTAITRKGMVSNRLNKFSIRKYTVGTASILVGTTLIFGLGNQEAKAAESTNKELNEATTSASDNQSSDKVDMQQLNQEDNTKNDNQKEMVSSQGNETTSNGNKSIEKESVQSTTGNKVEVSTAKSDEQASPKSTNEDLNTKQTISNQEGLQPDLLENKSVVNVQPTNEENKKVDAKTESTTLNVKSDAIKSNAETLVDNNSNSNNENNADIILPKSTAPKSLNTRMRMAAIQPNSTDSKNVNDLITSNTTLTVVDADNSKTIVPAQDYLSLKSQITVDDKVKSGDYFTIKYSDTVQVYGLNPEDIKNIGDIKDPNNGETIATAKHDTANNLITYTFTDYVDRFNSVKMGINYSIYMDADTIPVDKKDVPFSVTIGNQITTTTADITYPAYKEADNNSIGSAFTETVSHVGNVEDPGYYNQVVYVNPMDKDLKGAKLKVEAYHPKYPTNIGQINQNVTNIKIYRVPEGYTLNKGYDVNTNDLVDVTDEFKNKMTYGSNQSVNLDFGDITSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVILLNEKGEVIGTTKTDENGKYRFDNLDSGKYKVIFEKPTGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGNENSGSNNATLFGGLFAALGSLLLFGRRKKQNK

SEQ ID NO: 11 - DNA, 3426 bp, Staphylococcus sp.
  CDS (1)..(3426)
atgattaacagggataataaaaaggcaataacaaaaaagggtatgatttcaaatcgcttaaacaaattttcgattagaaagtatactgtaggaactgcatcgattttagtaggtacgacattgatttttggtctagggaaccaagaagctaaagctgctgaaaacactagtacagaaaatgcgaaacaagatgatgcaacgactagtgataataaagaagtagtgtcggaaactgaaaataattcgacaacagaaaatgattcaacaaatccaattaagaaagaaacaaatactgattcacaaccagaagctaaagaagaatcaactacatcaagtactcaacaacagcaaaataacgttacagctacaactgaaactaagcctcaaaacattgaaaaagaaaatgttaaaccttcaactgataaaactgcgacagaagatacatctgttattttagaagagaagaaagcaccaaattatacaaataacgatgtaactacaaaaccatctacaagtgaaattcaaacaaaaccaactacacctcaagaatctacaaatattgaaaattcacaaccgcaaccaacgccttcaaaagtagacaatcaagttacagatgcaactaatccaaaagaaccagtaaatgtgtcaaaagaagaacttaaaaataatcctgagaaattaaaagaattagttagaaatgataacaatacagatcgttcaactaaaccagttgctacagctccaacaagtgttgcaccaaaacgattaaatgcgaaaatgcgttttgcagttgcacaaccagcagcagttgcttcaaataatgtaaatgacttaattacagttacgaaacagacgatcaaagttggcgatggtaaagataatgtggcagcagcgcatgacggtaaagatattgaatatgatacagagtttacaattgacaataaagtcaaaaaaggcgatacaatgacgattaattatgataagaatgtaattccttcggatttaacagataaaaatgatcctatcgatattactgatccatcaggagaggtcattgccaaaggaacatttgataaagcgactaagcaaatcacatatacatttacagattatgtagataaatatgaagatataaaagcacgtttaactttatactcatatattgataagcaagcagtacctaatgaaactagtttgaatttaacgtttgcaacagcaggtaaagaaactagccaaaacgtttctgttgattatcaagacccaatggttcatggtgattcaaacattcaatctatctttacaaagttagatgaaaacaaacaaactattgaacaacaaatttatgttaatcctttgaaaaaaacagcaactaacactaaagttgatatagctggtagtcaagtagatgattatggaaatattaaactaggaaatggtagtaccattattgaccaaaatacagaaataaaagtttataaagttaaccctaatcaacaattgcctcaaagtaatagaatctatgattttagtcaatacgaagatgtaacaagtcaatttgataataaaaaatcatttagtaataatgtagcaacattggattttggtgatattaattcagcctatattatcaaagttgttagtaaatatacacctacatcagatggcgaactagatattgctcaaggtactagtatgagaacaactgataaatatggttattataattatgcaggatattcaaacttcatcgtaacttctaatgacactggcggtggcgacggtactgttaaacctgaagaaaagttatacaaaattggtgactatgtatgggaagacgttgataaagacggtgtccaaggtacagattcgaaagaaaagccaatggcaaacgttttagttacattaacttacccggacggtactacaaaatcagtaagaacagatgctaacggtcattatgaattcggtggtttgaaagacggagaaacttatacagttaaattcgaaacgccagctggatatcttccaacaaaagtaaatggaacaactgatggtgaaaaagactcaaatggtagttctataactgttaaaattaatggtaaagatgatatgtctttagacactggtttttataaagaacctaaatataatcttggtgactatgtatgggaagatacaaataaagatggtatccaagatgctaatgaacctggtatcaaagatgttaaggttacattaaaagatagtactggaaaagttattggtacaactactactgatgcctcgggtaaatataaatttacagatttagataatggtaactatacagtagaatttgaaacaccagcaggttacacgccaacggttaaaaatactacagctgaagataaagattctaatggtttaacaacaacaggtgtcattaaagatgcagataatatgacattagacagtggtttctataaaacaccaaaatacagtttaggtgattatgtttggtacgacagtaataaagacggtaaacaagattcaactgaaaaaggtatcaaagatgttaaagttactttattaaatgaaaaaggcgaagtaattggaacaactaaaacagatgaaaatggtaaatatcgtttcgataatttagatagcggtaaatacaaagttatttttgaaaagcctgctggcttaacacaaacagttacaaatacaactgaagatgataaagatgccgatggtggcgaagttgacgtaacaattacggatcatgatgatttcacacttgataacggatacttcgaagaagatacatcagacagtgattcagactcagacagtgattcagactcagacagcgactcagattcagacagtgattcagactcagatagcgattcagattcagacagcgactcagactcagatagcgactcagactcagacagcgactcagactcagatagcgactcagattcggacagcgattcagactcagatagcgactcagattcagacagcgattcagactcagatagcgactcagattcagacagtgactcagactcagatagcgactcagactcagacagtgactcagactcagacagcgattcagattcagatagcgactcagattcggacagtgattcagactcagatagcgactcagattcagacagcgactcagactcagatagcgactcagactcagacagtgattcagactcagatagcgattcggactcggatgcaggaaaacatacacctgttaaaccaatgagtactactaaagaccatcacaataaagcaaaagcattaccagaaacaggtagtgaaaataacggctcaaataacgcaacgttatttggtggattatttgcagcattaggttcattattgttattcggtcgtcgcaaaaaacaaaacaaataa

SEQ ID NO: 12 - protein, 1141 aa, Staphylococcus sp.
MINRDNKKAITKKGMISNRLNKFSIRKYTVGTASILVGTTLIFGLGNQEAKAAENTSTENAKQDDATTSDNKEVVSETENNSTTENDSTNPIKKETNTDSQPEAKEESTTSSTQQQQNNVTATTETKPQNIEKENVKPSTDKTATEDTSVILEEKKAPNYTNNDVTTKPSTSEIQTKPTTPQESTNIENSQPQPTPSKVDNQVTDATNPKEPVNVSKEELKNNPEKLKELVRNDNNTDRSTKPVATAPTSVAPKRLNAKMRFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPAGYLPTKVNGTTDGEKDSNGSSITVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTAEDKDSNGLTTTGVIKDADNMTLDSGFYKTPKYSLGDYVWYDSNKDGKQDSTEKGIKDVKVTLLNEKGEVIGTTKTDENGKYRFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 13 - DNA, 1052 bp, Staphylococcus sp.
  CDS (1)..(1050)
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaata

SEQ ID NO: 14 - protein, 350 aa, Staphylococcus sp.
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 15 - DNA, 1938 bp, Staphylococcus sp.
ttagtttttacgttttctaggtaatacgaatgcaacgatgctacttaaagctagtaatgccattaatggtaatgtcatatctttatttgattcttcaccagtttgtggtaatgattttgctttattttcttgtgtatttttattgttttggctttgagtgtgtccatcatttgtgtttttaatgtttgctttttgtaatggagcactatcttttgcttcgctagaacctgctgaagtttgaacaacatcttttgttgtttttgatgaagcagttgttggttttgcaacattttgagtcgtagatactaccttagttggagttgtactacttgattctacttcacctttagttggttttgtagcaggcgttttgtctttacctgactcactagatgcgtcattttctttttcaacacttggtaattgtttattgtcatctttttggctgtcttgtttttgtgattctttttcaacaggtgatggtgttggtttgctaggcgtagctggagtagcttccttcttagctgagttatcttgttgttcttttttgttagatttatcggtattggcttttgtaaatgcttctttatcaacgattctgacatggtattgtccatcataatcaatcgtttttacgtgaactttaacgatagcatcatatagagttttaccttcaacatatgggaaaataattgttctagtattatttttagcatctttgcttatagttctaacacgttgaccttcaaccatgaaatctttccagtaatcgtcattagtagtttccatgaccatatattttttgccgttaagcatacctgttttaatagggtgtttaacaaaagtatccatcatagattcgttattctcaacactttcataaacaacatattttgtatcttgtaaatcagtcattttttcatttgttggttgtacattttggaattcagtaatagctgatttcacttgctcatctaaagctttctttgtatcctctaatttcttcttgtactcagcctttaatttttcaggaagtttatcttgaattttatttaattcataaacttgtctttctagtgttttcgcttttttatatggcgctaataatttttcagctttataatcttcttcagttttgaatttatctgcactgttataaattggttgtgcgaattccattaatgtgtaatcgtatttttcttctttgttattgaagtgagttgaacttacaattttaacggcttttgttccatttgaaacagagaagcgaatgtaagcgtaatctttaacagtatcgtatgatactaatttaattggcaactttttgtcaccttcataaacttcaaattttctccaaaattgacctgattgtaatcctaattcaatttctggttttgaatcagtgaaaataactctagcaggtttaacagagctggcataatgataaaattgttgctcaccattttcttttttcatttcaaaatcaattggacgagagtttggtgcgctatgatctttatcttttattgcagggtttttaatcgcttctctaagttcctgattcaaaataggatatgtattgttagtggcttttgctgctggtttaactgcttttgtttccttaggggctttaacttctttaacttctttagcttcttttgtttcagaagtaggggcctcaacttctttattagatactgagacagcattagctactggtttagtttctggagctttttcagatgttgttgttggacttgcaactgcttcagtttttggttgtgcttctgtatttgtaccacctgtttcttcagctgctgcttgtgcttcgccatttgacattaataataaaagtgtactaatcgctacagatgcaacgcctagtgatgactttctaattgaataaaatgatttaaattctttttgctgtttgttcat

SEQ ID NO: 16 - protein, 645 aa, Staphylococcus sp.
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 17 - DNA, 1353 bp, Staphylococcus sp.
  CDS (1)..(1353)
ttgaaaaagaaaaacatttattcaattcgtaaactaggtgtaggtattgcatctgtaactttaggtacattacttatatctggtggcgtaacacctgctgcaaatgctgcgcaacacgatgaagctcaacaaaatgctttttatcaagtgttaaatatgcctaacttaaacgctgatcaacgtaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaatgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgtaacggcttcatccaaagccttaaagacgatccttcagtgagcaaagaaattttagcagaagctaaaaagctaaacgatgctcaagcaccaaaagaggaagacaacaaaaaacctggtaaagaagacggcaacaaacctggcaaagaagacggcaacaagcctggtaaagaagacaacaaaaaacctggtaaagaagacggcaacaagcctggtaaagaagacaacaacaaacctggcaaagaagacggcaacaagcctggtaaagaagacaacaacaagcctggtaaagaagacggcaacaagcctggtaaagaagacggcaacaaacctggtaaagaagacggcaacggagtacatgtcgttaaacctggtgatacagtaaatgacattgcaaaagcaaacggcactactgctgacaaaattgctgcagataacaaattagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataa

SEQ ID NO: 18 - protein, 450 aa, Staphylococcus sp.
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSVSKEILAEAKKLNDAQAPKEEDNKKPGKEDGNKPGKEDGNKPGKEDNKKPGKEDGNKPGKEDNNKPGKEDGNKPGKEDNNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 19 - DNA, 2634 bp, Staphylococcus sp.
  CDS (1)..(2634)
ttgaaaaaaagaattgattatttgtcgaataagcagaataagtattcgattagacgttttacagtaggtaccacatcagtaatagtaggggcaactatactatttgggataggcaatcatcaagcacaagcttcagaacaatcgaacgatacaacgcaatcttcgaaaaataatgcaagtgcagattccgaaaaaaacaatatgatagaaacacctcaattaaatacaacggctaatgatacatctgatattagtgcaaacacaaacagtgcgaatgtagatagcacaacaaaaccaatgtctacacaaacgagcaataccactacaacagagccagcttcaacaaatgaaacacctcaaccgacggcaattaaaaatcaagcaactgctgcaaaaatgcaagatcaaactgttcctcaagaagcaaattctcaagtagataataaaacaacgaatgatgctaatagcatagcaacaaacagtgagcttaaaaattctcaaacattagatttaccacaatcatcaccacaaacgatttccaatgcgcaaggaactagtaaaccaagtgttagaacgagagctgtacgtagtttagctgttgctgaaccggtagtaaatgctgctgatgctaaaggtacaaatgtaaatgataaagttacggcaagtaatttcaagttagaaaagactacatttgaccctaatcaaagtggtaacacatttatggcggcaaattttacagtgacagataaagtgaaatcaggggattattttacagcgaagttaccagatagtttaactggtaatggagacgtggattattctaattcaaataatacgatgccaattgcagacattaaaagtacgaatggcgatgttgtagctaaagcaacatatgatatcttgactaagacgtatacatttgtctttacagattatgtaaataataaagaaaatattaacggacaattttcattacctttatttacagaccgagcaaaggcacctaaatcaggaacatatgatgcgaatattaatattgcggatgaaatgtttaataataaaattacttataactatagttcgccaattgcaggaattgataaaccaaatggcgcgaacatttcttctcaaattattggtgtagatacagcttcaggtcaaaacacatacaagcaaacagtatttgttaaccctaagcaacgagttttaggtaatacgtgggtgtatattaaaggctaccaagataaaatcgaagaaagtagcggtaaagtaagtgctacagatacaaaactgagaatttttgaagtgaatgatacatctaaattatcagatagctactatgcagatccaaatgactctaaccttaaagaagtaacagaccaatttaaaaatagaatctattatgagcatccaaatgtagctagtattaaatttggtgatattactaaaacatatgtagtattagtagaagggcattacgacaatacaggtaagaacttaaaaactcaggttattcaagaaaatgttgatcctgtaacaaatagagactacagtattttcggttggaataatgagaatgttgtacgttatggtggtggaagtgctgatggtgattcagcagtaaatccgaaagacccaactccagggccgccggttgacccagaaccaagtccagacccagaaccagaaccaacgccagatccagaaccaagtccagacccagaaccggaaccaagcccagacccggatccggattcggattcagacagtgactcaggctcagacagcgactcaggttcagatagcgactcagaatcagatagcgattcggattcagacagtgattcagattcagacagcgactcagaatcagatagcgattcagaatcagatagcgactcagattcagatagcgattcagattcagatagcgattcagaatcagatagcgattcggattcagacagtgattcagattcagacagcgactcagaatcagatagcgactcagaatcagatagtgagtcagattcagacagtgactcggactcagacagtgattcagactcagatagcgattcagactcagatagcgattcagactcagacagcgattcagattcagacagcgactcagaatcagacagcgactcagactcagatagcgactcagactcagacagcgactcagattcagatagcgattcagactcagacagcgactcagactcagacagcgactcagactcagatagcgattcagactcagacagcgactcagattcagatagcgattcggactcagacagcgattcagattcagacagcgactcagactcggatagcgattcagattcagacagcgactcagactcggatagcgactcggattcagatagtgactccgattcaagagttacaccaccaaataatgaacagaaagcaccatcaaatcctaaaggtgaagtaaaccattctaataaggtatcaaaacaacacaaaactgatgctttaccagaaacaggagataagagcgaaaacacaaatgcaactttatttggtgcaatgatggcattattaggatcattactattgtttagaaaacgcaagcaagatcataaagaaaaagcgtaa

SEQ ID NO: 20 - protein, 877 aa, Staphylococcus sp.
MKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPTPDPEPSPDPEPEPSPDPDPDSDSDSDSGSDSDSGSDSDSESDSDSDSDSDSDSDSDSESDSDSESDSDSDSDSDSDSDSDSESDSDSDSDSDSDSDSDSESDSDSESDSESDSDSDSDSDSDSDSDSDSDSDSDSDSDSDSDSDSDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 21 - DNA, 684 bp, Staphylococcus sp.
  CDS (1)..(684)
ttgaaaaatattttaaaagtttttaatacaacgattttagcgttaattatcatcatcgcgacattcagtaattctgcaaatgccgcagatagcggtactttgaattatgaggtttacaaatacaataccaatgacacgtcaattgctaatgactattttaataaaccggcaaagtacattaagaaaaatggtaaattgtatgttcaaataactgtcaaccacagtcattggattactggaatgagtatcgaaggacataaagaaaatattattagtaaaaacactgccaaagatgaacgcacttctgaatttgaagtaagtaagttgaacggtaaaatagatggaaaaattgacgtttatatcgatgaaaaagtaaatggaaagccattcaaatatgaccatcattacaacattacatataaatttaatggaccaactgatgtagcaggtgctaatgcaccaggtaaagatgataaaaattctgcttcaggtagtgacaaaggatctgatggaacgactactggtcaaagtgaatctaatagttcgaataaagacaaagtagaaaatccacaaacaaatgctggtacacctgcatatatatatgcaataccagttgcatccttagcattattaatcgcaatcacattgtttgttagaaaaaaatctaaaggcaatgtggaataa

SEQ ID NO: 22 - protein, 227 aa, Staphylococcus sp.
MKNILKVFNTTILALIIIIATFSNSANAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGANAPGKDDKNSASGSDKGSDGTTTGQSESNSSNKDKVENPQTNAGTPAYIYAIPVASLALLIAITLFVRKKSKGNVE

SEQ ID NO: 23 - DNA, 1908 bp, Staphylococcus sp.
  CDS (1)..(1908)
atggctaaatatcgagggaaaccgtttcaattatatgtaaagttatcgtgttcgacaatgatggcgtcaagtatcattttaacgaatatcttgccgtacgatgcccaagctgcatctgaaaaggatactgaaatttcaaaagagatattatctaagcaagatttattagacaaagttgacaaagcaattcgtcaaattgagcaattaaaacagttatcggcttcatctaaagcacattataaagcacaactaaatgaagcgaaaacagcatcgcaaatagatgaaatcataaaacgagctaatgagttggatagcaaagaaaataaaagttctcacactgaaatgaacggtcaaagtgatatagacagtaaattagatcaattgcttaaagatttaaatgaggtttcttcaaatgttgataggggtcaacaaagtggcgaggacgatcttaatgcaatgaaaaatgatatgtcacaaacggctacaacaaaatatggagaaaaagatgataaaaatgatgaagcaatggtaaataaggcgttagaagacctagaccatttgaatcagcaaatacacaaatcgaaagatgcattgaaagatgcatcgaaagatccggcagtgtctacaacagatagtaatcatgaagtagctaaaacgccaaataatgatggttctggacatgttgtgttaaataaatttctttcaaatgaagagaatcaaagccatagtaatcaactcactgataaattacaaggaagcgataaaattaatcatgctatgattgaaaaattggctaaaagtaatgcctcaacgcaacattacacatatcataaactgaatacgttacaatctttagatcaacgtattgcaaatacgcaacttcctaaaaatcaaaaatcagacttaatgagcgaagtaaataagacgaaagagcgtataaaaagtcaacgaaatattattttggaagaacttgcacgtactgatgataaaaagtatgctacacaaagcattttagaaagtatatttaataaagacgaggcagataaaattctaaaagatatacgtgttgatggtaaaacagatcaacaaattgcagatcaaattactcgtcatattgatcaactatctctgacaacgagtgatgatttattaacgtcattgattgatcaatcacaagataagtcgctattgatttctcaaatcttacaaacgaaattaggaaaagctgaagcagataaattggctaaagattggacgaataaaggattatcaaatcgccaaatcgttgaccaattgaagaaacattttgcatcaactggcgacacgtcttcagatgatatattaaaagcaattttgaataatgccaaagataaaaagcaagcaattgaaacgattttagcaacacgtatagaaagacaaaaggcaaaattactggcagatttaattactaaaatagaaacagatcaaaataaaatttttaatttagttaaatcggcattgaatggtaaagcggatgatttattgaatttacaaaagagactcaatcaaacgaaaaaagatatagactatattttatcaccaatagtaaatcgtccaagtttactagatcgattgaataaaaatgggaaaacaacggatttaaataagttagcaaatttaatgaatcaaggatcaaatttattagacagtattccagatatacccacaccaaagccagaaaagacgttaacacttggtaaaggtaatggattgttaagtggattattaaatgctgatggtaatgtatctttgcctaaagcgggggaaacgataaaagaacattggttgccgatatctgtaattgttggtgcaatgggtgtactaatgatttggttatcacgacgcaataagttgaaaaataaagcataa

SEQ ID NO: 24 - protein, 635 aa, Staphylococcus sp.
MAKYRGKPFQLYVKLSCSTMMASSIILTNILPYDAQAASEKDTEISKEILSKQDLLDKVDKAIRQIEQLKQLSASSKAHYKAQLNEAKTASQIDEIIKRANELDSKENKSSHTEMNGQSDIDSKLDQLLKDLNEVSSNVDRGQQSGEDDLNAMKNDMSQTATTKYGEKDDKNDEAMVNKALEDLDHLNQQIHKSKDALKDASKDPAVSTTDSNHEVAKTPNNDGSGHVVLNKFLSNEENQSHSNQLTDKLQGSDKINHAMIEKLAKSNASTQHYTYHKLNTLQSLDQRIANTQLPKNQKSDLMSEVNKTKERIKSQRNIILEELARTDDKKYATQSILESIFNKDEADKILKDIRVDGKTDQQIADQITRHIDQLSLTTSDDLLTSLIDQSQDKSLLISQILQTKLGKAEADKLAKDWTNKGLSNRQIVDQLKKHFASTGDTSSDDILKAILNNAKDKKQAIETILATRIERQKAKLLADLITKIETDQNKIFNLVKSALNGKADDLLNLQKRLNQTKKDIDYILSPIVNRPSLLDRLNKNGKTTDLNKLANLMNQGSNLLDSIPDIPTPKPEKTLTLGKGNGLLSGLLNADGNVSLPKAGETIKEHWLPISVIVGAMGVLMIWLSRRNKLKNKA

SEQ ID NO: 25 - DNA, 2862 bp, Staphylococcus sp.
  CDS (1)..(2862)
atgaataataaaaagacagcaacaaatagaaaaggcatgataccaaatcgattaaacaaattttcgataagaaagtattctgtaggtactgcttcaattttagtagggacaacattgatttttgggttaagtggtcatgaagctaaagcggcagaacatacgaatggagaattaaatcaatcaaaaaatgaaacgacagccccaagtgagaataaaacaactgaaaaagttgatagtcgtcaactaaaagacaatacgcaaactgcaactgcagatcagcctaaagtgacaatgagtgatagtgcaacagttaaagaaactagtagtaacatgcaatcaccacaaaacgctacagctagtcaatctactacacaaactagcaatgtaacaacaaatgataaatcatcaactacatatagtaatgaaactgataaaagtaatttaacacaagcaaaaaacgtttcaactacacctaaaacaacgactattaaacaaagagctttaaatcgcatggcagtgaatactgttgcagctccacaacaaggaacaaatgttaatgataaagtacattttacgaacattgatattgcgattgataaaggacatgttaataaaacaacaggaaatactgaattttgggcaacttcaagtgatgttttaaaattaaaagcgaattacacaatcgatgattctgttaaagagggcgatacatttacttttaaatatggtcaatatttccgtccaggttctgtaagattaccttcacaaactcaaaatttatataatgcccaaggtaatattattgcaaaaggtatttacgatagtaaaacaaatacaacaacgtatacttttacgaattatgtagatcaatacacaaatgttagcggtagctttgaacaagtcgcatttgcgaaacgtgaaaatgcaacaactgataaaactgcttataaaatggaagtaactttaggtaatgatacatatagtaaagatgtcattgtcgattatggtaatcaaaaaggtcaacaacttatttcgagtacaaattatattaataatgaagatttgtcacgtaatatgactgtttatgtaaatcaacctaaaaagacctatacaaaagaaacatttgtaacaaatttaactggttataaatttaatccagatgctaaaaacttcaaaatttacgaagtgacagatcaaaatcaatttgtggatagtttcaccccagatacttcaaaacttaaagatgttactggtcaattcgatgttatttatagtaatgataataagacggcgacagtagatttattgaatggtcaatctagtagtgataaacagtacatcattcaacaagttgcttatccagataatagttcaacagataatgggaaaattgattatactttagaaacacaaaatggaaaaagtagttggtcaaacagttattcaaatgtgaatggctcatcaactgcaaatggcgaccaaaagaaatataatctaggtgactatgtatgggaagatacaaataaagatggtaaacaagatgccaatgaaaaagggattaaaggtgtttatgtcattcttaaagatagtaacggtaaagaattagatcgtacgacaacagatgaaaatggtaaatatcagttcactggtttaagcaatggaacttatagtgtagagttttcaacaccagccggttatacaccgacaactgcaaatgcaggtacagatgatgctgtagattctgatggactaactacaacaggtgtcattaaagacgctgacaacatgacattagatagtggattctacaaaacaccaaaatatagtttaggtgattatgtttggtacgacagtaataaagatggtaaacaagattcgactgaaaaaggaattaaaggtgttaaagttactttgcaaaacgaaaaaggcgaagtaattggtacaactgaaacagatgaaaatggtaaataccgctttgataatttagatagtggtaaatacaaagttatctttgaaaagcctgctggtttaactcaaacaggtacaaatacaactgaagatgataaagatgccgatggtggcgaagttgatgtaacaattacggatcatgatgatttcacacttgataatggctactacgaagaagaaacatcagatagtgactcagattcggacagcgattcagactcagatagcgactcagattcagatagtgactcagactcagatagcgactcagactcagatagcgactcagacagcgactcagactcagatagtgattcagattcggacagcgactcagattcagacagcgaatcagattcggatagcgactcagactcagatagcgactcagacagcgactcagattcagacagtgactcagactcagacagcgactcagattcagacagcgattcagattcggatagcgactcagattcagatagcgattcggactcagacaacgactcagattctgacagcgattcagactcagatagcgactcagattcagacagcgactcagattcagacagcgattcagattcagatagcgattcagattcagacagcgactcagattcagatagcgactcagactcagacagcgattcagactcagatagcgactcagacagcgattcagattcggatagcgattcagattcagatgcaggtaaacatactccgactaaaccaatgagtacggttaaagatcagcataaaacagctaaagcattaccagaaacaggtagtgaaaataataattcaaataatggcacattattcggtggattattcgcggcattaggatcattattgttattcggtcgtcgtaaaaaacaaaataaataa

SEQ ID NO: 26 - protein, 953 aa, Staphylococcus sp.
MNNKKTATNRKGMIPNRLNKFSIRKYSVGTASILVGTTLIFGLSGHEAKAAEHTNGELNQSKNETTAPSENKTTEKVDSRQLKDNTQTATADQPKVTMSDSATVKETSSNMQSPQNATASQSTTQTSNVTTNDKSSTTYSNETDKSNLTQAKNVSTTPKTTTIKQRALNRMAVNTVAAPQQGTNVNDKVHFTNIDIAIDKGHVNKTTGNTEFWATSSDVLKLKANYTIDDSVKEGDTFTFKYGQYFRPGSVRLPSQTQNLYNAQGNIIAKGIYDSKTNTTTYTFTNYVDQYTNVSGSFEQVAFAKRENATTDKTAYKMEVTLGNDTYSKDVIVDYGNQKGQQLISSTNYINNEDLSRNMTVYVNQPKKTYTKETFVTNLTGYKFNPDAKNFKIYEVTDQNQFVDSFTPDTSKLKDVTGQFDVIYSNDNKTATVDLLNGQSSSDKQYIIQQVAYPDNSSTDNGKIDYTLETQNGKSSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANAGTDDAVDSDGLTTTGVIKDADNMTLDSGFYKTPKYSLGDYVWYDSNKDGKQDSTEKGIKGVKVTLQNEKGEVIGTTETDENGKYRFDNLDSGKYKVIFEKPAGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSESDSDSDSDSDSDSDSDSDSDSDSDSDSDSDSDSDSDSDSDSDSDSDSDSDNDSDSDSDSDSDSDSDSDSDSDSDSDSDSDSDSDSDSDSDSDSDSDSDSDSDSDSDSDSDSDSDSDSDSDAGKHTPTKPMSTVKDQHKTAKALPETGSENNNSNNGTLFGGLFAALGSLLLFGRRKKQNK

SEQ ID NO: 27 - DNA, 2970 bp, Staphylococcus sp.
  CDS (1)..(2970)
atgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagaatacagatgcaagtaataaagatgtagttagtcaagcggttaatccaagtacgcctagaatgagagcatttagtttagcggcagtagctgcagatgcaccggcagctggcacagatattacgaatcagttgacagatgtgaaagttactattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgttgataataaagaaaatgtaacagctaatattactatgccagcttatattgaccctgaaaatgttacaaagacaggtaatgtgacattgacaactggcataggaaccaatactgctagtaagacagtattaatcgactatgagaaatatggacaattccataatttatcaattaaaggtacgattgatcaaatcgataaaacaaataatacgtatcgccaaacaatttatgtcaatccaagcggagataacgttgtgttacctgccttaacaggtaatttaattcctaatacaaagagtaatgcgttaatagatgcaaaaaacactgatattaaagtttatagagtcgataatgctaatgatttatctgaaagttattatgtgaatcctagcgattttgaagatgtaactaatcaagttagaatttcatttccaaatgctaatcaatacaaagtagaatttcctacggacgatgaccaaattacaacaccgtatattgtagttgttaatggccatattgatcctgctagtacaggtgatttagcactacgttcgacattttatggttatgattctaattttatatggagatctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattctggcagtgattctacatcagatagtggttcagattcagcgagtgattcagattcagcaagtgattcagactcagcgagtgattcagattcagcaagtgattcagattcagcaagtgattcagattcagcaagtgattcagactcagcaagtgattcagattcagcaagtgattcagattcagcaagcgattcagattcagcgagcgattcagattcagcgagcgattcagattcagcgagtgattccgactcagcgagcgattcagactcagatagtgactcagattccgatagcgattccgactcagatagcgactcagattcagacagcgattctgactcagacagcgattctgactcagacagtgactcagattccgatagcgattccgactcagacagtgactcagattccgatagcgattccgactcagacagtgactcagattccgatagcgattcagattccgacagtgattccgactcagatagcgattccgactcagatagcgactcagattcagacagcgattcagattcagacagcgattctgactcagacagtgactcagattccgatagcgattcagattcagacagtgattcagactcagatagcgattcagattccgacagtgactcagactcagacagcgattcagattccgatagcgattcagattccgacagtgactcagattccgatagtgactcggattcagcgagtgattcagattcagatagcgattcagaatcagatagtgactcagactcagacagtgattcagattcagatagtgactcagactcagacagcgattcagaatcagatagtgactccgattcagacagcgattcagaatcagatagtgactccgattcagatagcgattcggattcagcgagtgattcagactcaggtagtgactccgattcatcaagtgattcagattccgattcaacgagtgacacaggatcagacaacgactcagacagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcgaatacgtcactaatttggggattattagcatcattaggttcattactacttttcagaagaaaaaaagaaaataaagataagaaataa

SEQ ID NO: 28 - protein, 989 aa, Staphylococcus sp.
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQNTDASNKDVVSQAVNPSTPRMRAFSLAAVAADAPAAGTDITNQLTDVKVTIDSGTTVYPHQAGYVKLNYGFSVPNSAVKGDTFKITVPKELNLNGVTSTAKVPPIMAGDQVLANGVIDSDGNVIYTFTDYVDNKENVTANITMPAYIDPENVTKTGNVTLTTGIGTNTASKTVLIDYEKYGQFHNLSIKGTIDQIDKTNNTYRQTIYVNPSGDNVVLPALTGNLIPNTKSNALIDAKNTDIKVYRVDNANDLSESYYVNPSDFEDVTNQVRISFPNANQYKVEFPTDDDQITTPYIVVVNGHIDPASTGDLALRSTFYGYDSNFIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSASDSDSASDSDSASDSDSASDSDSASDSDSASDSDSASDSDSASDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSESDSDSDSDSDSDSDSDSDSDSDSESDSDSDSDSDSESDSDSDSDSDSDSASDSDSGSDSDSSSDSDSDSTSDTGSDNDSDSDSNSDSESGSNNNVVPPNSPKNGTNASNKNEAKDSKEPLPDTGSEDEANTSLIWGLLASLGSLLLFRRKKENKDKK

SEQ ID NO: 29 - DNA, 243 bp, Staphylococcus sp.
  CDS (1)..(240)
atgaatcagcacgtaaaagtaacatttgattttactaattataattacggcacatatgacttagcagtaccagcatatttaccgataaaaaacttaatagctttagtattggatagtttggacatttcaatatttgatgtcaatacacaaattaaagtgatgacgaaaggtcaattacttgttgaaaatgatcgactcattgattatcaaatcgctgatggagatattttgaagttactatag

SEQ ID NO: 30 - protein, 80 aa, Staphylococcus sp.
MNQHVKVTFDFTNYNYGTYDLAVPAYLPIKNLIALVLDSLDISIFDVNTQIKVMTKGQLLVENDRLIDYQIADGDILKLL

SEQ ID NO: 31 - DNA, 393 bp, Staphylococcus sp.
  CDS (1)..(393)
atgaattttaatgatattgaaacaatggttaagtcgaaatttaaagatattaaaaagcatgctgaagagattgcgcatgaaattgaagttcgttctggatatttaagaaaagctgaacaatataagcgattagaatttaatttgagttttgcactagatgatattgaaagcacagcaaaggacgtacaaactgcaaaatctagtgctaataaggacagtgtaactgttaagggaaaggcgcccaatacgttatatattgaaaaaagaaatttgatgaaacaaaagcttgaaatgttgggtgaagatatcgataaaaataaagaatccctccaaaaagctaaggaaattgctggcgaaaaggcaagtgaatattttaataaagcaatgaattaa

SEQ ID NO: 32 - protein, 130 aa, Staphylococcus sp.
MNFNDIETMVKSKFKDIKKHAEEIAHEIEVRSGYLRKAEQYKRLEFNLSFALDDIESTAKDVQTAKSSANKDSVTVKGKAPNTLYIEKRNLMKQKLEMLGEDIDKNKESLQKAKEIAGEKASEYFNKAMN

SEQ ID NO: 33 - DNA, 462 bp, Staphylococcus aureus
  CDS (1)..(462)
gcagaaattaataaacaaacaacatcacaaggtgtcacaactgaaaaaaataatggtatcgcagtgttagaacaagatgtgattacaccaacagttaaacctcaagcgaaacaagatattatccaagcagttacaactcgtaaacaacaaattaaaaagtcaaatgcatcattacaagatgaaaaagatgtagcaaatgataaaattggtaaaattgaaacaaaggcaattaaagatattgatgcagcaacaacaaatgcacaagtagaagccattaaaacaaaagcaatcaatgatattaatcaaactacacctgctacaacagctaaagcagcagctcttgaagaatttgacgaagttgttcaagcacaaattgatcaagcacctttaaatcctgatacaacaaatgaagaagtagcggaagctattgaacgtattaatgcagctaaagtttctggtgtt

SEQ ID NO: 34 - protein, 154 aa, Staphylococcus aureus
AEINKQTTSQGVTTEKNNGIAVLEQDVITPTVKPQAKQDIIQAVTTRKQQIKKSNASLQDEKDVANDKIGKIETKAIKDIDAATTNAQVEAIKTKAINDINQTTPATTAKAAALEEFDEVVQAQIDQAPLNPDTTNEEVAEAIERINAAKVSGV

SEQ ID NO: 35 - DNA, 2319 bp, Staphylococcus aureus
atgaaagctttattacttaaaacaagtgtatggctcgttttgctttttagtgtaatgggattatggcaagtctcgaacgcggctgagcagcatacaccaatgaaagcacatgcagtaacaacgatagacaaagcaacaacagataagcaacaagtaccgccaacaaaggaagcggctcatcattctggcaaagaagcggcaaccaacgtatcagcatcagcgcagggaacagctgatgatacaaacagcaaagtaacatccaacgcaccatctaacaaaccatctacagtagtttcaacaaaagtaaacgaaacacgcgacgtagatacacaacaagcctcaacacaaaaaccaactcacacagcaacgttcaaattatcaaatgctaaaacagcatcactttcaccacgaatgtttgctgctaatgcaccacaaacaacaacacataaaatattacatacaaatgatatccatggccgactagccgaagaaaaagggcgtgtcatcggtatggctaaattaaaaacagtaaaagaacaagaaaagcctgatttaatgttagacgcaggagacgccttccaaggtttaccactttcaaaccagtctaaaggtgaagaaatggctaaagcaatgaatgcagtaggttatgatgctatggcagtcggtaaccatgaatttgactttggatacgatcagttgaaaaagttagagggtatgttagacttcccgatgctaagtactaacgtttataaagatggaaaacgcgcgtttaagccttcaacgattgtaacaaaaaatggtattcgttatggaattattggtgtaacgacaccagaaacaaagacgaaaacaagacctgaaggcattaaaggcgttgaatttagagatccattacaaagtgtgacagcggaaatgatgcgtatttataaagacgtagatacatttgttgttatatcacatttaggaattgatccttcaacacaagaaacatggcgtggtgattacttagtgaaacaattaagtcaaaatccacaattgaagaaacgtattacagttattgatggtcattcacatacagtacttcaaaatggtcaaatttataacaatgatgcattggcacaaacaggtacagcacttgcgaatatcggtaagattacatttaattatcgcaatggagaggtatcgaatattaaaccgtcattgattaatgttaaagacgttgaaaatgtaacaccgaacaaagcattagctgaacaaattaatcaagctgatcaaacatttagagcacaaactgcagaggtaattattccaaacaataccattgatttcaaaggagaaagagatgacgttagaacgcgtgaaacaaatttaggaaacgcgattgcagatgctatggaagcgtatggcgttaagaatttctctaaaaagactgactttgccgtgacaaatggtggaggtattcgtgcctctatcgcaaaaggtaaggtgacacgctatgatttaatctcagtattaccatttggaaatacgattgcgcaaattgatgtaaaaggttcagacgtctggacggctttcgaacatagtttaggcgcaccaacaacacaaaaggacggtaagacagtgttaacagcgaatggcggtttactacatatctctgattcaatccgtgtttactatgatataaataaaccgtctggcaaacgaattaatgctattcaaattttaaataaagagacaggtaagtttgaaaatattgatttaaaacgtgtatatcacgtaacgatgaatgacttcacagcatcaggtggcgacggatatagtatgttcggtggtcctagagaagaaggtatttcattagatcaagtactagcaagttatttaaaaacagctaacttagctaagtatgatacgacagaaccacaacgtatgttattaggtaaaccagcagtaagtgaacaaccagctaaaggacaacaaggtagcaaaggtagtaagtctggtaaagatacacaaccaattggtgacgacaaagtgatggatccagcgaaaaaaccagctccaggtaaagttgtattgttgctagcgcatagaggaactgttagtagcggtacagaaggttctggtcgcacaatagaaggagctactgtatcaagcaagagtgggaaacaattggctagaatgtcagtgcctaaaggtagcgcgcatgagaaacagttaccaaaaactggaactaatcaaagttcaagcccagaagcgatgtttgtattattagcaggtataggtttaatcgcgactgtacgacgtagaaaagctagctaa

SEQ ID NO: 36 - protein, 745 aa, Staphylococcus aureus
AEQHTPMKAHAVTTIDKATTDKQQVPPTKEAAHHSGKEAATNVSASAQGTADDTNSKVTSNAPSNKPSTVVSTKVNETRDVDTQQASTQKPTHTATFKLSNAKTASLSPRMFAANAPQTTTHKILHTNDIHGRLAEEKGRVIGMAKLKTVKEQEKPDLMLDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKITFNYRNGEVSNIKPSLINVKDVENVTPNKALAEQINQADQTFRAQTAEVIIPNNTIDFKGERDDVRTRETNLGNAIADAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDINKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSKSGKDTQPIGDDKVMDPAKKPAPGKVVLLLAHRGTVSSGTEGSGRTIEGATVSSKSGKQLARMSVPKGSAHEKQLPKTGTNQSSSPEAMFVLLAGIGLIATVRRRKAS

SEQ ID NO: 37 - protein, 322 aa, Staphylococcus sp.
MMKMKTRIVSSVTTTLLLGSILMNPVANAADSDINIKTGTTDIGSNTTVKTGDLVTYDKENGMHKKVFYSFIDDKNHNKKILVIRTKGTIAGQYRVYSEEGANKSGLAWPSAFKVQLQLPDNEVAQISDYYPRNSIDTKEYMSTLTYGFNGNVTGDDSGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKATKQQTNIDVIYERVRDDYQLHWTSTNWKGTNTKDKWTDRSSERYKIDWEKEEMTN

SEQ ID NO: 38 - protein, 10419 aa, Staphylococcus sp.
MNYRDKIQKFSIRKYTVGTFSTVIATLVFLGFNTSQAHAAETNQPASVVKQKQQSNNEQTENRESQVQNSQNSQNGQSLSATHENEQPNISQANLVDQKVAQSSTTNDEQPASQNVNTKKDSATAATTQPDKEQSKHKQNESQSANKNGNDNRAAHVENHEANVVTASDSSDNGNVQHDRNELQAFFDANYHDYRFIDRENADSGTFNYVKGIFDKINTLLGSNDPINNKDLQLAYKELEQAVALIRTMPQRQQTSRRSNRIQTRSVESRAAEPRSVSDYQNANSSYYVENANDGSGYPVGTYINASSKGAPYNLPTTPWNTLKASDSKEIALMTAKQTGDGYQWVIKFNKGHAPHQNMIFWFALPADQVPVGRTDFVTVNSDGTNVQWSHGAGAGANKPLQQMWEYGVNDPHRSHDFKIRNRSGQVIYDWPTVHIYSLEDLSRASDYFSEAGATPATKAFGRQNFEYINGQKPAESPGVPKVYTFIGQGDASYTISFKTQGPTVNKLYYAAGGRALEYNQLFMYSQLYVESTQDHQQRLNGLRQVVNRTYRIGTTKRVEVSQGNVQTKKVLESTNLNIDDFVDDPLSYVKTPSNKVLGFYSNNANTNAFRPGGAQQLNEYQLSQLFTDQKLQEAARTRNPIRLMIGFDYPDAYGNSETLVPVNLTVLPEIQHNIKFFKNDDTQNIAEKPFSKQAGHPVFYVYAGNQGNASVNLGGSVTSIQPLRINLTSNENFTDKDWQITGIPRTLHIENSTNRPNNARERNIELVGNLLPGDYFGTIRFGRKEQLFEIRVKPHTPTITTTAEQLRGTALQKVPVNISGIPLDPSALVYLVAPTNQTTNGGSEADQIPSGYTILATGTPDGVHNTITIRPQDYVVFIPPVGKQIRAVVYYNKVVASNMSNAVTILPDDIPPTINNPVGINAKYYRGDEVNFTMGVSDRHSGIKNTTITTLPNGWTSNLTKADKNNGSLSITGRVSMNQAFNSDITFKVSATDNVNNTTNDSQSKHVSIHVGKISEDAHPIVLGNTEKVVVVNPTAVSNDEKQSIITAFMNKNQNIRGYLASTDPVTVDNNGNVTLHYRDGSSTTLDATNVMTYEPVVKPEYQTVNAAKTATVTIAKGQSFSIGDIKQYFTLSNGQPIPSGTFTNITSDRTIPTAQEVSQMNAGTQLYHITATNAYHKDSEDFYISLKIIDVKQPEGDQRVYRTSTYDLTTDEISKVKQAFINANRDVITLAEGDISVTNTPNGANVSTITVNINKGRLTKSFASNLANMNFLRWVNFPQDYTVTWTNAKIANRPTDGGLSWSDDHKSLIYRYDATLGTQITTNDILTMLKATTTVPGLRNNITGNEKSQAEAGGRPNFRTTGYSQSNATTDGQRQFTLNGQVIQVLDIINPSNGYGGQPVTNSNTRANHSNSTVVNVNEPAANGAGAFTIDHVVKSNSTHNASDAVYKAQLYLTPYGPKQYVEHLNQNTGNTTDAINIYFVPSDLVNPTISVGNYTNHQVFSGETFTNTITANDNFGVQSVTVPNTSQITGTVDNNHQHVSATAPNVTSATNKTINLLATDTSGNTATTSFNVTVKPLRDKYRVGTSSTAANPVRIANISNNATVSQADQTTIINSLTFTETVPNRSYARASANEITSKTVSNVSRTGNNANVTVTVTYQDGTTSTVTVPVKHVIPEIVAHSHYTVQGQDFPAGNGSSASDYFKLSNGSDIADATITWVSGQAPNKDNTRIGEDITVTAHILIDGETTPITKTATYKVVRTVPKHVFETARGVLYPGVSDMYDAKQYVKPVNNSWSTNAQHMNFQFVGTYGPNKDVVGISTRLIRVTYDNRQTEDLTILSKVKPDPPRIDANSVTYKAGLTNQEIKVNNVLNNSSVKLFKADNTPLNVTNITHGSGFSSVVTVSDALPNGGIKAKSSISMNNVTYTTQDEHGQVVTVTRNESVDSNDSATVTVTPQLQATTEGAVFIKGGDGFDFGHVERFIQNPPHGATVAWHDSPDTWKNTVGNTHKTAVVTLPNGQGTRNVEVPVKVYPVANAKAPSRDVKGQNLTNGTDAMNYITFDPNTNTNGITAAWANRQQPNNQQAGVQHLNVDVTYPGISAAKRVPVTVNVYQFEFPQTTYTTTVGGTLASGTQASGYAHMQNATGLPTDGFTYKWNRDTTGTNDANWSAMNKPNVAKVVNAKYDVIYNGHTFATSLPAKFVVKDVQPAKPTVTETAAGAITIAPGANQTVNTHAGNVTTYADKLVIKRNGNVVTTFTRRNNTSPWVKEASAATVAGIAGTNNGITVAAGTFNPADTIQVVATQGSGETVSDEQRSDDFTVVAPQPNQATTKIWQNGHIDITPNNPSGHLINPTQAMDIAYTEKVGNGAEHSKTINVVRGQNNQWTIANKPDYVTLDAQTGKVTFNANTIKPNSSITITPKAGTGHSVSSNPSTLTAPAAHTVNTTEIVKDYGSNVTAAEINNAVQVANKRTATIKNGTAMPTNLAGGSTTTIPVTVTYNDGSTEEVQESIFTKADKRELITAKNHLDDPVSTEGKKPGTITQYNNAMHNAQQQINTAKTEAQQVINNERATPQQVSDALTKVRAAQTKIDQAKALLQNKEDNSQLVTSKNNLQSSVNQVPSTAGMTQQSIDNYNAKKREAETEITAAQRVIDNGDATAQQISDEKHRVDNALTALNQAKHDLTADTHALEQAVQQLNRTGTTTGKKPASITAYNNSIRALQSDLTSAKNSANAIIQKPIRTVQEVQSALTNVNRVNERLTQAINQLVPLADNSALKTAKTKLDEEINKSVTTDGMTQSSIQAYENAKRAGQTESTNAQNVINNGDATDQQIAAEKTKVEEKYNSLKQAIAGLTPDLAPLQTAKTQLQNDIDQPTSTTGMTSASIAAFNEKLSAARTKIQEIDRVLASHPDVATIRQNVTAANAAKSALDQARNGLTVDKAPLENAKNQLQHSIDTQTSTTGMTQDSINAYNAKLTAARNKIQQINQVLAGSPTVEQINTNTSTANQAKSDLDHARQALTPDKAPLQTAKTQLEQSINQPTDTTGMTTASLNAYNQKLQAARQKLTEINQVLNGNPTVQNINDKVTEANQAKDQLNTARQGLTLDRQPALTTLHGASNLNQAQQNNFTQQINAAQNHAALETIKSNITALNTAMTKLKDSVADNNTIKSDQNYTDATPANKQAYDNAVNAAKGVIGETTNPTMDVNTVNQKAASVKSTKDALDGQQNLQRAKTEATNAITHASDLNQAQKNALTQQVNSAQNVQAVNDIKQTTQSLNTAMTGLKRGVANHNQVVQSDNYVNADTNKKNDYNNAYNHANDIINGNAQHPVITPSDVNNALSNVTSKEHALNGEAKLNAAKQEANTALGHLNNLNNAQRQNLQSQINGAHQIDAVNTIKQNATNLNSAMGNLRQAVADKDQVKRTEDYADADTAKQNAYNSAVSSAETIINQTTNPTMSVDDVNRATSAVTSNKNALNGYEKLAQSKTDAARAIDALPHLNNAQKADVKSKINAASNIAGVNTVKQQGTDLNTAMGNLQGAINDEQTTLNSQNYQDATPSKKTAYTNAVQAAKDILNKSNGQNKTKDQVTEAMNQVNSAKNNLDGTRLLDQAKQTAKQQLNNMTHLTTAQKTNLTNQINSGTTVAGVQTVQSNANTLDQAMNTLRQSIANKDATKASEDYVDANNDKQTAYNNAVAAAETIINANSNPEMNPSTITQKAEQVNSSKTALNGDENLAAAKQNAKTYLNTLTSITDAQKNNLISQITSATRVSGVDTVKQNAQHLDQAMASLQNGINNESQVKSSEKYRDADTNKQQEYDNAITAAKAILNKSTGPNTAQNAVEAALQRVNNAKDALNGDAKLIAAQNAAKQHLGTLTHITTAQRNDLTNQISQATNLAGVESVKQNANSLDGAMGNLQTAINDKSGTLASQNFLDADEQKRNAYNQAVSAAETILNKQTGPNTAKTAVEQALNNVNNAKHALNGTQNLNNAKQAAITAINGASDLNQKQKDALKAQANGAQRVSNAQDVQHNATELNTAMGTLKHAIADKTNTLASSKYVNADSTKQNAYTTKVTNAEHIISGTPTVVTTPSEVTAAANQVNSAKQELNGDERLREAKQNANTAIDALTQLNTPQKAKLKEQVGQANRLEDVQTVQTNGQALNNAMKGLRDSIANETTVKTSQNYTDASPNNQSTYNSAVSNAKGIINQTNNPTMDTSAITQATTQVNNAKNGLNGAENLRNAQNTAKQNLNTLSHLTNNQKSAISSQIDRAGHVSEVTATKNAATELNTQMGNLEQAIHDQNTVKQSVKFTDADKAKRDAYTNAVSRAEAILNKTQGANTSKQDVEAAIQNVSSAKNALNGDQNVTNAKNAAKNALNNLTSINNAQKRDLTTKIDQATTVAGVEAVSNTSTQLNTAMANLQNGINDKTNTLASENYHDADSDKKTAYTQAVTNAENILNKNSGSNLDKTAVENALSQVANAKGALNGNHNLEQAKSNANTTINGLQHLTTAQKDKLKQQVQQAQNVAGVDTVKSSANTLNGAMGTLRNSIQDNTATKNGQNYLDATERNKTNYNNAVDSANGVINATSNPNMDANAINQIATQVTSTKNALDGTHNLTQAKQTATNAIDGATNLNKAQKDALKAQVTSAQRVANVTSIQQTANELNTAMGQLQHGIDDENATKQTQKYRDAEQSKKTAYDQAVAAAKAILNKQTGSNSDKAAVDRALQQVTSTKDALNGDAKLAEAKAAAKQNLGTLNHITNAQRTDLEGQINQATTVDGVNTVKTNANTLDGAMNSLQGSINDKDATLRNQNYLDADESKRNAYTQAVTAAEGILNKQTGGNTSKADVDNALNAVTRAKAALNGADNLRNAKTSATNTIDGLPNLTQLQKDNLKHQVEQAQNVAGVNGVKDKGNTLNTAMGALRTSIQNDNTTKTSQNYLDASDSNKNNYNTAVNNANGVINATNNPNMDANAINGMANQVNTTKAALNGAQNLAQAKTNATNTINNAHDLNQKQKDALKTQVNNAQRVSDANNVQHTATELNSAMTALKAAIADKERTKASGNYVNADQEKRQAYDSKVTNAENIISGTPNATLTVNDVNSAASQVNAAKTALNGDNNLRVAKEHANNTIDGLAQLNNAQKAKLKEQVQSATTLDGVQTVKNSSQTLNTAMKGLRDSIANEATIKAGQNYTDASPNNRNEYDSAVTAAKAIINQTSNPTMEPNTITQVTSQVTTKEQALNGARNLAQAKTTAKNNLNNLTSINNAQKDALTRSIDGATTVAGVNQETAKATELNNAMHSLQNGINDETQTKQTQKYLDAEPSKKSAYDQAVNAAKAILTKASGQNVDKAAVEQALQNVNSTKTALNGDAKLNEAKAAAKQTLGTLTHINNAQRTALDNEITQATNVEGVNTVKAKAQQLDGAMGQLETSIRDKDTTLQSQNYQDADDAKRTAYSQAVNAAATILNKTAGGNTPKADVERAMQAVTQANTALNGIQNLDRAKQAANTAITNASDLNTKQKEALKAQVTSAGRVSAANGVEHTATELNTAMTALKRAIADKAETKASGNYVNADANKRQAYDEKVTAAENIVSGTPTPTLTPADVTNAATQVTNAKTQLNGNHNLEVAKQNANTAIDGLTSLNGPQKAKLKEQVGQATTLPNVQTVRDNAQTLNTAMKGLRDSIANEATIKAGQNYTDASQNKQTDYNSAVTAAKAIIGQTTSPSMNAQEINQAKDQVTAKQQALNGQENLRTAQTNAKQHLNGLSDLTDAQKDAVKRQIEGATHVNEVTQAQNNADALNTAMTNLKNGIQDQNTIKQGVNFTDADEAKRNAYTNAVTQAEQILNKAQGPNTSKDGVETALENVQRAKNELNGNQNVANAKTTAKNALNNLTSINNAQKEALKSQIEGATTVAGVNQVSTTASELNTAMSNLQNGINDEAATKAAQKYTDADREKQTAYNDAVTAAKTLLDKTAGSNDNKAAVEQALQRVNTAKTALNGDERLNEAKNTAKQQVATMSHLTDAQKANLTSQIESGTTVAGVQGIQANAGTLDQAMNQLRQSIASKDATKSSEDYQDANADLQNAYNDAVTNAEGIISATNNPEMNPDTINQKASQVNSAKSALNGDEKLAAAKQTAKSDIGRLTDLNNAQRTAANAEVDQAPNLAAVTAAKNKATSLNTAMGNLKHALAEKDNTKRSVNYTDADQPKQQAYDTAVTQAEAITNANGSNANETQVQAALNQLNQAKNDLNGDNKVAQAKESAKRALASYSNLNNAQSTAAISQIDNATTVAGVTAAQNTANELNTAMGQLQNGINDQNTVKQQVNFTDADQGKKDAYTNAVTNAQGILDKAHGQNMTKAQVEAALNQVTTAKNALNGDANVRQAKSDAKANLGTLTHLNNAQKQDLTSQIEGATTVNGVNGVKTKAQDLDGAMQRLQSAIANKDQTKASENYIDADPTKKTAFDNAITQAESYLNKDHGANKDKQAVEQAIQSVTSTENALNGDANLQRAKTEAIQAIDNLTHLNTPQKTALKQQVNAAQRVSGVTDLKNSATSLNNAMDQLKQAIADHDTIVASGNYTNASPDKQGAYTDAYNAAKNIVNGSPNVITNAADVTAATQRVNNAETGLNGDTNLATAKQQAKDALRQMTHLSDAQKQSITGQIDSATQVTGVQSVKDNATNLDNAMNQLRNSIANKDDVKASQPYVDADRDKQNAYNTAVTNAENIINATSQPTLDPSAVTQAANQVSTNKTALNGAQNLANKKQETTANINQLSHLNNAQKQDLNTQVTNAPNISTVNQVKTKAEQLDQAMERLINGIQDKDQVKQSVNFTDADPEKQTAYNNAVTAAENIINQANGTNANQSQVEAALSTVTTTKQALNGDRKVTDAKNNANQTLSTLDNLNNAQKGAVTGNINQAHTVAEVTQAIQTAQELNTAMGNLKNSLNDKDTTLGSQNFADADPEKKNAYNEAVHNAENILNKSTGTNVPKDQVEAAMNQVNATKAALNGTQNLEKAKQHANTAIDGLSHLTNAQKEALKQLVQQSTTVAEAQGNEQKANNVDAAMDKLRQSIADNATTKQNQNYTDASQNKKDAYNNAVTTAQGIIDQTTSPTLDPTVINQAAGQVSTTKNALNGNENLEAAKQQASQSLGSLDNLNNAQKQTVTDQINGAHTVDEANQIKQNAQNLNTAMGNLKQAIADKDATKATVNFTDADQAKQQAYNTAVTNAENISKANGNATQAEVEQAIKQVNAAKQALNGNANVQHAKDEATALINSSNDLNQAQKDALKQQVQNATTVAGVNNVKQTAQELNNAMTQLKQGIADKEQTKADGNFVNADPDKQNAYNQAVAKAEALISATPDVVVTPSEITAALNKVTQAKNDLNGNTNLATAKQNVQHAIDQLPNLNQAQRDEYSKQITQATLVPNVNAIQQAATTLNDAMTQLKQGIANKAQIKGSENYHDADTDKQTAYDNAVTKAEELLKQTTNPTMDPNTIQQALTKVNDTNQALNGNQKLADAKQDAKTTLGTLDHLNDAQKQALTTQVEQAPDIATVNNVKQNAQNLNNAMTNLNNALQDKTETLNSINFTDADQAKKDAYTNAVSHAEGILSKANGSNASQTEVEQAMQRVNEAKQALNGNDNVQRAKDAAKQVITNANDLNQAQKDALKQQVDAAQTVANVNTIKQTAQDLNQAMTQLKQGIADKDQTKANGNFVNADTDKQNAYNNAVAHAEQIISGTPNANVDPQQVAQALQQVNQAKGDLNGNHNLQVAKDNANTAIDQLPNLNQPQKTALKDQVSHAELVTGVNAIKQNADALNNAMGTLKQQIQANSQVPQSVDFTQADQDKQQAYNNAANQAQQIANGIPTPVLTPDTVTQAVTTMNQAKDALNGDEKLAQAKQEALANLDTLRDLNQPQRDALRNQINQAQALATVEQTKQNAQNVNTAMSNLKQGIANKDTVKASENYHDADADKQTAYTNAVSQAEGIINQTTNPTLNPDEITRALTQVTDAKNGLNGEAKLATEKQNAKDAVSGMTHLNDAQKQALKGQIDQSPEIATVNQVKQTATSLDQAMDQLSQAINDKAQTLADGNYLNADPDKQNAYKQAVAKAEALLNKQSGTNEVQAQVESITNEVNAAKQALNGNDNLANAKQQAKQQLANLTHLNDAQKQSFESQITQAPLVTDVTTINQKAQTLDHAMELLRNSVADNQTTLASEDYHDATAQRQNDYNQAVTAANNIINQTTSPTMNPDDVNGATTQVNNTKVALDGDENLAAAKQQANNRLDQLDHLNNAQKQQLQSQITQSSDIAAVNGHKQTAESLNTAMGNLINAIADHQAVEQRGNFINADTDKQTAYNTAVNEAAAMINKQTGQNANQTEVEQAITKVQTTLQALNGDHNLQVAKTNATQAIDALTSLNDPQKTALKDQVTAATLVTAVHQIEQNANTLNQAMHGLRQSIQDNAATKANSKYINEDQPEQQNYDQAVQAANNIINEQTATLDNNAINQAATTVNTTKAALHGDVKLQNDKDHAKQTVSQLAHLNNAQKHMEDTLIDSETTRTAVKQDLTEAQALDQLMDALQQSIADKDATRASSAYVNAEPNKKQSYDEAVQNAESIIAGLNNPTINKGNVSSATQAVISSKNALDGVERLAQDKQTAGNSLNHLDQLTPAQQQALENQINNATTRGEVAQKLTEAQALNQAMEALRNSIQDQQQTEAGSKFINEDKPQKDAYQAAVQNAKDLINQTNNPTLDKAQVEQLTQAVNQAKDNLHGDQKLADDKQHAVTDLNQLNGLNNPQRQALESQINNAATRGEVAQKLAEAKALDQAMQALRNSIQDQQQTESGSKFINEDKPQKDAYQAAVQNAKDLINQTGNPTLDKSQVEQLTQAVTTAKDNLHGDQKLARDQQQAVTTVNALPNLNHAQQQALTDAINAAPTRTEVAQHVQTATELDHAMETLKNKVDQVNTDKAQPNYTEASTDKKEAVDQALQAAESITDPTNGSNANKDAVDQVLTKLQEKENELNGNERVAEAKTQAKQTIDQLTHLNADQIATAKQNIDQATKLQPIAELVDQATQLNQSMDQLQQAVNEHANVEQTVDYTQADSDKQNAYKQAIADAENVLKQNANKQQVDQALQNILNAKQALNGDERVALAKTNGKHDIDQLNALNNAQQDGFKGRIDQSNDLNQIQQIVDEAKALNRAMDQLSQEITDNEGRTKGSTNYVNADTQVKQVYDETVDKAKQALDKSTGQNLTAKQVIKLNDAVTAAKKALNGEERLNNRKAEALQRLDQLTHLNNAQRQLAIQQINNAETLNKASRAINRATKLDNAMGAVQQYIDEQHLGVISSTNYINADDNLKANYDNAIANAAHELDKVQGNAIAKAEAEQLKQNIIDAQNALNGDQNLANAKDKANAFVNSLNGLNQQQQDLAHKAINNADTVSDVTDIVNNQIDLNDAMETLKHLVDNEIPNAEQTVNYQNADDNAKTNFDDAKRLANTLLNSDNTNVNDINGAIQAVNDAIHNLNGDQRLQDAKDKAIQSINQALANKLKEIEASNATDQDKLIAKNKAEELANSIINNINKATSNQAVSQVQTAGNHAIEQVHANEIPKAKIDANKDVDKQVQALIDEIDRNPNLTDKEKQALKDRINQILQQGHNGINNAMTKEEIEQAKAQLAQALQDIKDLVKAKEDAKQDVDKQVQALIDEIDQNPNLTDKEKQALKYRINQILQQGHNDINNALTKEEIEQAKAQLAQALQDIKDLVKAKEDAKNAIKALANAKRDQINSNPDLTPEQKAKALKEIDEAEKRALQNVENAQTIDQLNRGLNLGLDDIRNTHVWEVDEQPAVNEIFEATPEQILVNGELIVHRDDIITEQDILAHINLIDQLSAEVIDTPSTATISDSLTAKVEVTLLDGSKVIVNVPVKVVEKELSVVKQQAIESIENAAQQKINEINNSVTLTLEQKEAAIAEVNKLKQQAIDHVNNAPDVHSVEEIQQQEQAHIEQFNPEQFTIEQAKSNAIKSIEDAIQHMIDEIKARTDLTDKEKQEAIAKLNQLKEQAIQAIQRAQSIDEISEQLEQFKAQMKAANPTAKELAKRKQEAISRIKDFSNEKINSIRNSEIGTADEKQAAMNQINEIVLETIRDINNAHTLQQVEAALNNGIARISAVQIVTSDRAKQSSSTGNESNSHLTIGYGTANHPFNSSTIGHKKKLDEDDDIDPLHMRHFSNNFGNVIKNAIGVVGISGLLASFWFFIAKRRRKEDEEEELEIRDNNKDSIKETLDDTKHLPLLFAKRRRKEDEEDVTVEEKDSLNNGESLDKVKHTPFFLPKRRRKEDEEDVEVTNENTDEKVLKDNEHSPLLFAKRRKDKEEDVETTTSIESKDEDVPLLLAKKKNQKDNQSKDKKSASKNTSKKVAAKKKKKKAKKNKK

SEQ ID NO: 39 - protein, 636 aa, Staphylococcus sp.
MKKQIISLGALAVASSLFTWDNKADAIVTKDYSGKSQVNAGSKNGTLIDSRYLNSALYYLEDYIIYAIGLTNKYEYGDNIYKEAKDRLLEKVLREDQYLLERKKSQYEDYKQWYANYKKENPRTDLKMANFHKYNLEELSMKEYNELQDALKRALDDFHREVKDIKDKNSDLKTFNAAEEDKATKEVYDLVSEIDTLVVSYYGDKDYGEHAKELRAKLDLILGDTDNPHKITNERIKKEMIDDLNSIIDDFFMETKQNRPKSITKYNPTTHNYKTNSDNKPNFDKLVEETKKAVKEADDSWKKKTVKKYGETETKSPVVKEEKKVEEPQAPKVDNQQEVKTTAGKAEETTQPVAQPLVKIPQGTITGEIVKGPEYPTMENKTVQGEIVQGPDFLTMEQSGPSLSNNYTNPPLTNPILEGLEGSSSKLEIKPQGTESTLKGTQGESSDIEVKPQATETTEASQYGPRPQFNKTPKYVKYRDAGTGIREYNDGTFGYEARPRFNKPSETNAYNVTTHANGQVSYGARPTYKKPSETNAYNVTTHANGQVSYGARPTQNKPSKTNAYNVTTHGNGQVSYGARPTQNKPSKTNAYNVTTHANGQVSYGARPTYKKPSKTNAYNVTTHADGTATYGPRVTK

SEQ ID NO: 40 - protein, 628 aa, Staphylococcus sp.
MSDRFIKFNDEQLDAKQVMMLQDLARLLLKNEQTQVKIQKFPYYNPVQNVLITSWFWSHRPSHIEMAGLKTDVMLAAYGYHMMDVQIVNEVVQDKTFKHPKFYQQLFKLLEDMRVLNSIKVERPSTAKLIDLRLDTRISYTESQIKVYRTKTQYTDLLFLYLEHAFLSQDFFDIPSIHSDLDDILVNMFLYLPNFFQNQNSEDNMYLAQRIMYQVDDILKEDMLNEYYYLPKTLYNTLASPEFDDLKRTDASQVDGQDDTSEDDDNESEKADSKSADSESKGGAYLEMELHEGQNSETLGNDEAREGDATDDMTDMMTKKGKGSNDTLNREEGDAVGQSQAFQLDGVNKNVEIKWQIPEIEPQYVLEYQESKQDVQYEIKDLIQIIKKTIEREQRDARFNLTKGRLQKDLINWFIDDQYKLFYKKQDLSKSFDATFTLLIDASASMHDKMAETKKGVVLFHETLKALNIKHEILSFSEDAFDSDEHAQPNIINEIINYDYSTFEKDGPRIMALEPQDDNRDGVAIRVASERLMRRNQHQRFLIVFSDGEPSAFNYSQDGIIDTYEAVEMSRKFGIEVFNVFLSQDPITEDVEQTIHNIYGQYAIFVEGVAHLPGHLSPLLKKLLLKSL

SEQ ID NO: 41 - protein, 780 aa, Bacillus anthracis
MKKSKKMLAGATLAIGVMAPQVLPATAHADEKTGESTVNLRILETSDIHVNLMNYDYYQTKTDNKVGLVQTATLVNKAREEAKNSVLFDDGDALQGTPLGDYVANKINDPKKPVDPSYTHPLYRLMNLMKYDVISLGNHEFNYGLDYLNKVISKTEFPVINSNVYKDDKDNNEENDQNYFKPYHVFEKEVEDESGQKQKVKIGVMGFVPPQVMNWDKANLEGKVKAKDIVETAKKMVPKMKAEGADVIVALAHSGVDKSGYNVGMENASYYLTEVPGVDAVLMGHSHTEVKDVFNGVPVVMPGVFGSNLGIIDMQLKKVNGKWEVQKEQSKPQLRPIADSKGNPLVQSDQNLVNEIKDDHQATIDYVNTAVGKTTAPINSYFSLVQDDPSVQLVTNAQKWYVEKLFAENGQYSKYKGIPVLSAGAPFKAGGRNGATYYTDIPAGTLAIKNVADLYVYPNTLYAVKVNGAQVKEWLEMSAGQFNQIDPKKTEEQPLVNIGYPTYNFDILDGLKYEIDVTQPAKYDKDGKVVNANTNRIINMTYEGKPVADNQEFIVATNNYRGSSQTFPGVSKGEVVYQSQDETRQIIVKYMQETPVIDPAADKNWAFKPIVADKLNTTFDSSPNAQKYIKKDGNISYVGPSENEFAKYAIDITKKNDDDKETGGENPTIPPTGEGDNGENPTTPPTGEGNNGENPTTPPTGEGNNGGNPTTPSTDEGNNAGSGQTTTDNQNSKETTTVSENKEERDLPKTGTNVASTIGAGLAFIGAGFLLLFRRKKANR

SEQ ID NO: 42 - DNA, 30 bp, Artificial
  Synthetic primer
tttcccgggacgatccagctctaatcgctg

SEQ ID NO: 43 - DNA, 41 bp, Artificial
  Synthetic primer
tttgagctcaaagcaaatagataatcgagaaatataaaaag

SEQ ID NO: 44 - DNA, 27 bp, Artificial
  Synthetic primer
tttgagctcagttgctccagccagcat

SEQ ID NO: 45 - DNA, 28 bp, Artificial
  Synthetic primer
tttgaattcaaacggattcattccagcc

SEQ ID NO: 46 - DNA, 30 bp, Artificial
  Synthetic primer
tacgaattcgacttggcaggcaattgaaaa

SEQ ID NO: 47 - DNA, 29 bp, Artificial
  Synthetic primer
tgtgaattcttagctagcttttctacgtc

SEQ ID NO: 48 - DNA, 30 bp, Artificial
  Synthetic primer
tcgggatccgctgagcagcatacaccaatg

SEQ ID NO: 49 - DNA, 36 bp, Artificial
  Synthetic primer
tgtggatccttattgattaatttgttcagctaatgc

SEQ ID NO: 50 - protein, 340 aa, Staphylococcus aureus
MKKKLLVLTMSTLFATQLINSNHANASVTESVDKKFVVPESGINKIIPTYNEFKKAPKVNVSNLTDNKNFVASEDKLKKISDPSAASKIVDKNFVVPESKLGNIVPEYKEINNRVNVATNNPASQQVDKHFVAKGPEVNRFITQNKVNHHFITTQTHYKKVITSYKSTHVHKHVNHATDSINKHFIVKPSEAPRYTHPSQSLMINHYFAVPGYHAHKFVTPGHASIKINHFCVVPQINSFKVIPPYGHNSHRMHVPSFQNNTTATHQNAKVNKAYDYKYFYSYKVVKGVKKYFSFSQSNGYKIGKPSLNIKNVNYQYAVPSYSPTNYVPEFKGSLPAPRV

SEQ ID NO: 51 - protein, 339 aa, Staphylococcus aureus
MKKKLFVLTMSTLFATQLINSNHANASTESVDKNFVVPESGINKIIPTYDEFKKAPKVNVGSLADNKNFVASEDKLSKIADPSAASKIVDKNFVVPESKLGNIVPEYKEINNRVNVATNNPASQQVDKHFVAKGPEVNRFITQNKVNHPFITTQTHYKKVITSYKSTHVHKHVNHATGSINKHFIVKPSEAPRYTQPSQSLMINHYFAVPGYHAHKFVTPGHASIKINHFCVVPQINSFKVIPPYGHNSHRMHVPSFQNNTTATHQNAKVKKAYDYKYFYSYKVVKGVKKYFSFSQSNGYKIGEPSLNIKNVNYQYAVPSYSPTHYVPEFKGSIPAPRV

SEQ ID NO: 52 - protein, 340 aa, Staphylococcus aureus
MKKKLLVLTMSTLFATQLINSNHAKASVTESVDKKFVVPESGINKIIPAYDEFKNSPKVNVSNLTDNKNFVVSEDKLNKIVDSSAASKIVDKNFAVPESKLGNIVPEYKEINNRVNVATNNPASQQVDKHFVAKGPEVNRFITQNKVNHHFITTQTHYKKVITSYKSTHVHKHVNHAKDSINKHFIVKPSESPRYTHPSQSLIIKHHFAVPGYHAHKFVTPGHASIKINHFCVVPQINSFKVIPPYGHNSHRMHVPSFQNNTTATHQNAKVNKAYDYKYFYSYKVVKGVKKYFSFSQSNGYKIGKPSLNIKNVNYQYAVPSYSPTHYVPEFKGSLPAPRV

SEQ ID NO: 53 - protein, 340 aa, Staphylococcus aureus
MKKKLLVLTMSTLFATQIMNSNHAKASVTESVDKKFVVPESGINKIIPTYNEFKKAPKVNVGNLADNKNFVASEDKLNKIVDSSAASKIVDKNFAVPESKLGNIVPEYKEINNRVNVATNNPASQQVDKHFVAKGPEVNRFITQNKVNHHFITTQTHYKKVITSYKSTHVHKHVNHAKDSINKHFIVKPSESPRYTHPSQSLIIKHHFAVPGYHAHKFVTPGHASIKINHFCVVPQINSFKVIPPYGHNSHRMHVPSFQNNTTATHQNAKVNKAYDYKYFYSYKVVKGVKKYFSFSQSNGYKIGKPSLNIKNVNYQYAVPSYSPTHYVPEFKGSLPAPRV